(19) United States
(12) Patent Application Publication
(10) Pub. No.: US 2024/0141363 A1
(43) Pub. Date: May 2, 2024

(54) SIGNAL PEPTIDES FOR INCREASED PROTEIN SECRETION

(71) Applicants: BOEHRINGER INGELHEIM RCV GMBH & CO KG, Wien (AT); VALIDOGEN GMBH, Grambach (AT); LONZA LTD, Visp (CH)

(72) Inventors: Richard Zahrl, Wien (AT); Oezge Ata Aykol, Wien (AT); Diethard Mattanovich, Wien (AT); Brigitte Gasser, Wien (AT)

(21) Appl. No.: 18/276,559

(22) PCT Filed: Feb. 11, 2022
(86) PCT No.: PCT/EP2022/053425

§ 371 Date: Aug. 9, 2023

(30) Foreign Application Priority Data
Feb. 12, 2021 (EP) 21156986.8

(51) Int. Cl. C12N 15/62; C07K 14/39; C07K 16/00; C12N 9/10

(52) U.S. Cl. CPC C12N 15/625; C07K 14/39; C07K 16/00; C12N 9/1048; C07K 2319/036; C12N 2800/102

(57) ABSTRACT

The present invention relates to a nucleic acid molecule encoding a fusion protein comprising a secretion signal comprising (i) a signal peptide sequence originating from a KRE1 protein or a signal peptide sequence originating from a SWP1 protein; and optionally (ii) an α-mating factor (MFα) pro-sequence, and a protein of interest. The present invention further relates to a secretion signal as defined herein, an expression cassette comprising said nucleic acid molecule as well as recombinant eukaryotic host cells comprising said nucleic acid molecule or expression cassette. Further encompassed is a method of manufacturing a protein of interest in a eukaryotic host cell and a method of increasing the secretion of a protein of interest from a eukaryotic host cell. Further provided is the use of the secretion signal for increasing the secretion of a recombinant protein of interest from a eukaryotic host cell and the use of the recombinant host cell for manufacturing a recombinant protein of interest.

### CROSS-REFERENCE TO RELATED APPLICATIONS

The present application claims the benefit of priority of EP Patent Application No. 21 156 986.8 filed on 12 Feb. 2021, the content of which is hereby incorporated by reference in its entirety for all purposes.

### TECHNICAL FIELD OF THE INVENTION

The present invention relates to a nucleic acid molecule encoding a fusion protein comprising a secretion signal comprising (i) a signal peptide sequence originating from a KRE1 protein or a signal peptide sequence originating from a SWP1 protein; and optionally (ii) an α-mating factor (MFα) pro-sequence, and a protein of interest. The present invention further relates to a secretion signal as defined herein, an expression cassette comprising said nucleic acid molecule as well as recombinant eukaryotic host cells comprising said nucleic acid molecule or expression cassette. Further encompassed is a method of manufacturing a protein of interest in a eukaryotic host cell and a method of increasing the secretion of a protein of interest from a eukaryotic host cell. Further provided is the use of the secretion signal for increasing the secretion of a recombinant protein of interest from a eukaryotic host cell and the use of the recombinant host cell for manufacturing a recombinant protein of interest.

### BACKGROUND OF THE INVENTION

Yeasts in general and Pichia pastoris (P. pastoris, synonym: Komagataella phaffii) in particular are popular expression systems for the secretion of recombinant proteins. The initial and crucial step in secretion is the translocation of the recombinant protein into the endoplasmic reticulum (ER). This process is directed by an N-terminal secretion signal fused to the recombinant protein. The signal sequence specifies either a co-translational or post-translational targeting route to the ER on the conventional secretion pathway (Ng et al., 1996). The most commonly used secretion signal in P. pastoris is the Saccharomyces cerevisiae α-mating prepro-leader (MFα) (Lin-Cereghino et al., 2013). This signal mediates post-translational translocation in S. cerevisiae and most likely in P. pastoris too (Fitzgerald and Glick, 2014; Ng et al., 1996). Other secretion signals are continually added to the repertoire and tested with different recombinant proteins.

As the biogenesis of many mammalian proteins may require co-translational translocation, the MFα signal sequence could, in fact, be suboptimal, and it may be preferable to use a co-translational signal sequence (Ng et al., 1996). Today, mammalian antibodies have become the dominant product class within the biopharmaceutical market (Ecker et al., 2015). Antibodies are known to be co-translationally translocated in their native environment (Feige et al., 2010). A trend toward development of smaller antigen-binding fragments (e.g. Fab, scFv and VHH) is also evident (Nelson and Reichert, 2009; Walsh, 2014). In particular, Fab fragments are sometimes inefficiently secreted and therefore only reach low production titers (Looser et al., 2015; Pfeffer et al., 2011). This may be due to the post-translational signal sequence MFα, which has already been reported as causing a bottleneck in translocation (Fitzgerald and Glick, 2014; Zahrl et al., 2018). W02018165589A2 and W02018165594 disclose a recombinant secretion signal comprising a MFα pro-leader originating from Saccharomyces cerevisiae and a signal peptide other than MFα pre-sequence originating from Saccharomyces cerevisiae. Fitzgerald et al. (Microb Cell Fact 13, 125 (2014) disclose a hybrid secretion signal consisting of the Ost1 signal sequence followed by the MFα pro-sequence.

Consequently, there is still a need for secretion signals that increase the secretion of various proteins such as antibodies. The technical problem therefore is to comply with this need.

### SUMMARY OF THE INVENTION

The technical problem is solved by the subject-matter as defined in the claims. The inventors surprisingly found that the secretion of fusion proteins comprising the signal peptide sequence, signal peptide or pre-sequence (all terms can be used interchangeably) originating from a KRE1 protein (internal designation SP14) or a SWP1 protein (internal designation SP4), optionally in combination with an α-mating factor (MFα) pro-sequence, is significantly increased. In other words, a protein comprising the secretion signal of the invention will be secreted at a higher rate while the secretion signal will be cleaved off (see Examples 6-8).

Accordingly, the present invention relates to a nucleic acid molecule encoding a fusion protein comprising from N-terminus to C-terminus
(a) a secretion signal, the secretion signal comprising
   (I) (i) a signal peptide sequence originating from a KRE1 protein or a signal peptide sequence originating from a SWP1 protein; and
      (ii) an α-mating factor (MFα) pro-sequence;
   or
      (II) a signal peptide sequence originating from a KRE1 protein or a signal peptide sequence originating from a SWP1 protein; and
(b) a protein of interest.

The present invention relates to a nucleic acid molecule encoding a fusion protein comprising from N-terminus to C-terminus
(a) a secretion signal, the secretion signal comprising
   (i) a signal peptide sequence originating from a KRE1 protein or a signal peptide sequence originating from a SWP1 protein; and
   (ii) an α-mating factor (MFα) pro-sequence; and
(b) a protein of interest.

In particular, the present invention provides for nucleic acid molecule encoding a fusion protein comprising from N-terminus to C-terminus
(a) a secretion signal, the secretion signal comprising
   (i) a signal peptide sequence originating from a KRE1 protein; and
   (ii) an α-mating factor (MFα) pro-sequence; and
(b) a protein of interest.

The present invention also relates to a nucleic acid molecule encoding a fusion protein comprising from N-terminus to C-terminus
(a) a secretion signal, the secretion signal comprising
   (i) a signal peptide sequence originating from a SWP1 protein; and
   (ii) an α-mating factor (MFα) pro-sequence; and
(b) a protein of interest.

The present invention further relates to a nucleic acid molecule encoding a fusion protein comprising from N-terminus to C-terminus
(a) a secretion signal, the secretion signal comprising
   (i) a signal peptide sequence originating from a KRE1 protein or a signal peptide sequence originating from a SWP1 protein; and
(b) a protein of interest.

In particular, the present invention further relates to a nucleic acid molecule encoding a fusion protein comprising from N-terminus to C-terminus
(a) a secretion signal, the secretion signal comprising
   (i) a signal peptide sequence originating from a KRE1 protein; and
(b) a protein of interest.

The present invention further relates to a nucleic acid molecule encoding a fusion protein comprising from N-terminus to C-terminus
(a) a secretion signal, the secretion signal comprising
   (i) a signal peptide sequence originating from a SWP1 protein; and
(b) a protein of interest.

The present invention further relates to a nucleic acid molecule encoding a fusion protein comprising from N-terminus to C-terminus
(a) a secretion signal, the secretion signal consisting of
   (i) a signal peptide sequence originating from a KRE1 protein or a
   signal peptide sequence originating from a SWP1 protein; and
(b) a protein of interest.

In particular, the present invention further relates to a nucleic acid molecule encoding a fusion protein comprising from N-terminus to C-terminus
(a) a secretion signal, the secretion signal consisting of
   (i) a signal peptide sequence originating from a KRE1 protein; and
(b) a protein of interest.

The present invention further relates to a nucleic acid molecule encoding a fusion protein comprising from N-terminus to C-terminus
(a) a secretion signal, the secretion signal consisting of
   (i) a signal peptide sequence originating from a SWP1 protein; and
(b) a protein of interest.

It is envisaged that the secretion signal increases secretion of said protein of interest from a eukaryotic host cell in comparison to said eukaryotic host cell expressing the nucleic acid molecule as defined herein but comprising a wild type Saccharomyces cerevisiae α-mating factor secretion signal (such as SEQ ID NO: 4) instead of the secretion signal as defined herein.

The signal peptide sequence originating from a KRE1 protein may comprise SEQ ID NO: 1 or a functional homolog thereof. The signal peptide sequence originating from a KRE1 protein may consist of SEQ ID NO: 1 or a functional homolog thereof. Specifically, the functional homolog of SEQ ID NO: 1 comprises at least 80%, or at least 85%, or at least 90%, or at least 94%, or at least 95%, sequence identity to SEQ ID NO: 1. Specifically, the functional homolog comprises one, two or three point mutations as compared to SEQ ID NO: 1. Specifically, the functional homolog has the function of a signal peptide in a eukaryotic host cell e.g., fungal or yeast host cells, such as Komagataella host cells.

The signal peptide sequence originating from a SWP1 protein may comprise SEQ ID NO: 2 or 52, or a functional homolog thereof. The signal peptide sequence originating from a SWP1 protein may consist of SEQ ID NO: 2 or 52, or a functional homolog thereof. Specifically, the functional homolog of SEQ ID NO: 2 or SEQ ID NO: 52 comprises at least 80%, or at least 85%, or at least 90%, or at least 94%, or at least 95% sequence identity to the respective SEQ ID NO: 2 or SEQ ID NO: 52. Specifically, the functional homolog comprises one, two or three point mutations as compared to the respective SEQ ID NO: 2 or SEQ ID NO: 52. Specifically, the functional homolog has the function of a signal peptide in a eukaryotic host cell e.g., fungal or yeast host cells, such as Komagataella host cells.

The MFα pro-sequence may comprise any one of SEQ ID NO: 3 or 53 or 74-80, or a functional homolog thereof. The MFα pro-sequence may consist of any one of SEQ ID NO: 3 or 53 or 74-80, or a functional homolog thereof, preferably SEQ ID NO: 3 or 53 or a functional homolog thereof. Specifically, the functional homolog of any one of SEQ ID NO: 3, 53 or 74-80 comprises at least 80%, or at least 85%, or at least 90%, or at least 95%, or at least 98% sequence identity to the respective SEQ ID NO: 3, 53 or 74-80. Specifically, the functional homolog comprises one, two or three point mutations as compared to the respective SEQ ID NO: 3, 53, or 74-80. Specifically, the functional homolog has the function of a pro-sequence in a eukaryotic host cell e.g., fungal or yeast host cells, such as Komagataella or Saccharomyces host cells. The MFα pro-sequence preferably comprises Ser at a position corresponding to position 23 of SEQ ID NO: 53 and/or Glu at a position corresponding to position 64 of SEQ ID NO: 53.

The protein of interest may be selected from the group consisting of an antibody such as a chimeric, humanized or human antibody, or a bispecific antibody, or an antigen-binding antibody fragment such as Fab or F(ab)₂, single chain antibodies such as scFv, single domain antibodies such as VHH fragments of camelid or heavy chain antibodiesor domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.

In another aspect, the present invention relates to a secretion signal as defined herein. In particular, the present invention relates to a secretion signal comprising (i) a signal peptide sequence originating from a KRE1 protein or a signal peptide sequence originating from a SWP1 protein; and (ii) an α-mating factor (MFα) pro-sequence. More particularly, the present invention relates to a secretion signal comprising (i) a signal peptide sequence originating from a KRE1 protein and (ii) an α-mating factor (MFα) pro-sequence. More particularly, the present invention relates to a secretion signal comprising (i) a signal peptide sequence originating from a SWP1 protein; and (ii) an α-mating factor (MFα) pro-sequence. The present invention further particularly relates to a secretion signal comprising a signal peptide sequence originating from a KRE1 protein. Further particularly, the present invention relates to a secretion signal comprising a signal peptide sequence originating from a SWP1 protein. The present invention further particularly relates to a secretion signal consisting of a signal peptide sequence originating from a KRE1 protein. Further particularly, the present invention relates to a secretion signal consisting of a signal peptide sequence originating from a SWP1 protein.

In still another aspect, the present invention further relates to an expression cassette comprising the nucleic acid molecule of the invention and a promoter operably linked thereto. The expression cassette may be comprised in a vector, preferably an expression vector, or be integrated within a chromosome in particular an artificial chromosome.

In another aspect, the present invention further provides for a recombinant eukaryotic host cell comprising the nucleic acid molecule of the invention, the vector of the invention or the expression cassette of the invention. It is herein understood that the recombinant eukaryotic host cell engineered with such nucleic acid molecule or expression cassette is genetically engineered to incorporate the respective nucleic acid molecule, vector or expression cassette. The recombinant eukaryotic host cell may be genetically engineered to comprise such nucleic acid molecule, vector or expression cassette within the host cell genome.

The recombinant eukaryotic host cell may be a fungal or yeast host cell, preferably a yeast host cell, selected from the group consisting of Komagataella phaffii (Pichia pastoris), Hansenula polymorpha, Saccharomyces cerevisiae, Kluyveromyces lactis, Yarrowia lipolytica, Pichia methanolica, Candida boidinii, Komagataella spp. and Schizosaccharomyces pombe, or a fungal host cell selected from Trichoderma reesei, or Aspergillus niger.

The host cell may be engineered to overexpress one or more component(s) of a signal recognition particle (SRP).

The present invention further relates to a method of producing a protein of interest by culturing the host cell of the invention under conditions to express the nucleic acid molecule of the invention and to secrete the protein of interest upon cleavage of the secretion signal, and isolating the protein of interest from the host cell culture, and optionally purifying and optionally modifying and optionally formulating the protein of interest.

In another aspect, the present invention further relates to a method of manufacturing a protein of interest in a eukaryotic host cell, comprising

(i) genetically engineering the eukaryotic host cell with the nucleic acid molecule of the invention or with the expression cassette or the vector of the invention, and optionally genetically engineering the eukaryotic host cell to overexpress one or more component(s) of a signal recognition particle (SRP);

(ii) culturing the genetically engineered host cell under conditions to express the nucleic acid molecule and optionally the one or more component(s) of the SRP, and to secrete the protein of interest upon cleavage of the secretion signal,

(iii) optionally isolating the protein of interest from the cell culture,

(iv) optionally purifying the protein of interest,

(v) optionally modifying the protein of interest, and

(vi) optionally formulating the protein of interest.

In still another aspect, the present invention further relates to a method of increasing the secretion of a protein of interest from a eukaryotic host cell, comprising expressing in said eukaryotic host cell the nucleic acid molecule of the invention and optionally engineering the eukaryotic host cell to overexpress one or more component(s) of a signal recognition particle (SRP), thereby increasing the secretion of said protein of interest in comparison to said host cell expressing the nucleic acid molecule of the invention but comprising a wild type Saccharomyces cerevisiae α-mating factor secretion signal (such as SEQ ID NO: 4) instead of the secretion signal described herein.

The method of increasing the secretion of a protein of interest from a eukaryotic host cell, may additionally comprise

(i) engineering said host cell to incorporate an expression construct to express a nucleic acid molecule of the invention, and optionally genetically engineering the host cell to overexpress the one or more component(s) of a signal recognition particle (SRP),

(ii) culturing said host cell under conditions to express said nucleic acid molecule and optionally to overexpress the one or more component(s) of the SRP and to secrete the protein of interest upon cleavage of the secretion signal,

(iii) optionally isolating the protein of interest from the cell culture,

(iv) optionally purifying the protein of interest,

(v) optionally modifying the protein of interest, and

(vi) optionally formulating the protein of interest.

The nucleic acid molecule of the invention may be integrated in a chromosome of said host cell or contained in an expression cassette, vector or plasmid, which does not integrate into a chromosome of said host cell.

In another aspect, the present invention further relates to a use of the secretion signal as described herein (e.g., as part of or within the nucleic acid molecule of the invention) for increasing the secretion of a protein of interest from a eukaryotic host cell. The secretion signal may further increase secretion of said protein of interest from the eukaryotic host cell in comparison to said eukaryotic host cell expressing the fusion protein as described herein comprising the wild type Saccharomyces cerevisiae α-mating factor secretion signal (such as SEQ ID NO: 4) instead of the secretion signal as defined by the present invention.

In still another aspect, the present invention relates to the use of the recombinant host cell of the invention for manufacturing a protein of interest.

### BRIEF DESCRIPTION OF THE DRAWINGS

The invention will be better understood with reference to the detailed description when considered in conjunction with the non-limiting examples and the accompanying drawings, respectively. The Figures show:

FIG. 1: Immunofluorescent Anti-His staining of SPx-VHH(His6) clones. A: MFα secretion signal, B: SWP1 (SP4), C: KRE1 (SP14): The cells were viewed in a fluorescence microscope with the appropriate filter cubes. The fluorescence, DIC and the merged images are shown. The images were brightness and contrast adjusted.

### DETAILED DESCRIPTION OF THE INVENTION

The present invention is described in detail in the following and is also illustrated by the appended examples and figures.

The inventors surprisingly found that the secretion and yield of fusion proteins comprising the signal peptide (signal peptide sequence in the following) of KRE1 (also designated as SP14 herein) or SWP1 (also designated as SP4 herein) in particular when combined with a pro-sequence, such as the pro-sequence of the α-mating factor, thereby forming the inventive secretion signal, are significantly increased (see Examples 6-8), while other signal peptides or combinations did not improve the secretion of proteins of interest. Fusion proteins comprising the secretion signal according to the present invention are thus more efficiently secreted. The inventors further found that by additionally overexpressing the proteins of the signal recognition particle (SRP), the secretion and yield of proteins of interest is increased even further (see Examples 6-8).

As outlined herein above, fusion proteins comprising the inventive secretion signal and a protein of interest are more efficiently secreted by recombinant host cells when compared to a eukaryotic host cell expressing a fusion protein as defined herein comprising a wild type Saccharomyces cerevisiae α-mating factor secretion signal (such as SEQ ID NO: 4) instead of the secretion signal as defined herein, i.e. the protein of interest comprised in the fusion protein of the invention is secreted while the secretion signal is cleaved off during secretion. Accordingly, the present invention surprisingly demonstrates that a fusion protein comprising from N-terminus to C-terminus (a) a secretion signal, the secretion signal comprising (i) a signal peptide sequence originating from a KRE1 protein or a signal peptide sequence originating from a SWP1 protein; and (ii) optionally an α-mating factor (MFα) pro-sequence; and (b) a protein of interest provides superior properties, e.g. an increased secretion of the protein of interest (see Examples 6-8).

The wording “from N-terminus to C-terminus” does not necessarily exclude that the secretion signal, including the signal peptide sequence and the α-mating factor (MFα) pro-sequence, and the protein of interest are separated by one or more amino acids. These one or more amino acids may be a linker or linker sequences. A “linker sequence” (also referred to as a “spacer sequence” or “linker”) is an amino acid sequence that is introduced between the secretion signal as defined herein and the protein of interest as defined herein. The “linker sequence” may also be an amino acid sequence that is introduced between the signal peptide sequence and the α-mating factor (MFα) pro-sequence and/or between the α-mating factor (MFα) pro-sequence and the protein of interest. Preferably, there is however no linker between the signal peptide sequence and the α-mating factor (MFα) pro-sequence. There are a great variety of possible linker sequences and it is within the knowledge of the person skilled in the art to choose a suitable linker sequence based on, e.g., the size, sequence and physical properties (such as hydrophobicity) of the polypeptide of the invention. Linker sequences can be composed of flexible residues like glycine and serine or rather rigid residues as alanine-proline repeats. It may be preferred that the linker sequence does not adopt a secondary structure (such as an α-helical structure or a β-sheet) in order to ensure maximal flexibility. A linker sequence can be a protease cleavage site, such as recognized by a specific protease e.g., by a member of the subtilisin/kexin-like proprotein convertase (PC) family. The term “linker sequence” or “linker” as used herein refers to any amino acid sequence that does not interfere with the function of elements being linked. A linker may connect e.g., nucleotide sequences, or amino acid sequences. A linker may be used to engineer appropriate amounts of flexibility. Preferably, a linker is short, e.g., 1-20 nucleotides or amino acids or even more and are typically flexible. Amino acid linkers commonly used consist of a number of glycine, serine, and optionally alanine, in any order. Such linkers usually have a length of at least any one of 1, 2, 3, 4, 5, 6, 7, 8, 9, 10, 11, 12, 13, 14, 15, or 20 amino acids, as required. Preferably, the linker used herein comprises 1 to 12 amino acid residues, preferably it is a short linker consisting of up to 5 amino acids. Preferably the linker used herein is any one of a GS, GGSGG, GSAGSAAGSG, (GS)n, wherein “n” is any number between 1 and 10, GSGSGSG, GSG or a GGGGS (“G4S”) linker or any combination thereof. In some embodiments, the linker comprises one or more units, repeats or copies of a motif, such as for example GS, GSG or G4S.

Alternatively or additionally, the fusion protein may comprise a cleavage site between the MFα pro-sequence and the protein of interest. The fusion protein may also comprise one or more further cleavage site(s), for example for cleaving of a tag, as described below. The cleavage site may be a protease cleavage site, such as recognized by a specific protease. Thus, the term “protease cleavage site” comprises the recognition site of a specific protease, which is an amino acid sequence that is recognized specifically by a protease, and the site between the 2 amino acids of a protein where the cleavage takes place. The protease may be selected from the group consisting of TEV (tobacco etch virus) protease, Chymotrypsin, enterokinase, Pepsin, neutrophil elastase, Proteinase K, Thermolysin, Thrombin and Trypsin. The site where the cleavage takes place by a specific protease is preferably between the N-terminal amino acid of the protein of interest as defined herein and the C-terminal amino acid of a N-terminally fused tag or of a N-terminally fused α-mating factor (MFα) pro-sequence as defined herein or between the C-terminal amino acid of the protein of interest as defined herein and the N-terminal amino acid of a C-terminally fused tag or of a C-terminally fused α-mating factor (MFα) pro-sequence. The recognition site can be adjacent to the respective site where the cleavage takes place outside the protein of interest (POI) as defined herein (within the tag). In more detail, a protease useful for cleavage of a tag is preferably selected from the group consisting of endopeptidase like factor Xa, thrombin, TEV (tobacco etch virus protease), cysteinyl aspartate-specific protease (caspase) and enterokinase. Thus, the recognition site used can be selected from the group consisting of a factor Xa, thrombin, TEV (tobacco etch virus protease), cysteinyl aspartate-specific protease (caspase) and enterokinase recognition sites but also any other protease cleavage site for proteases known to be useful for the cleavage of tags from the protein. A preferred cleavage site for caspase-2 is VDVAD.

As shown in the Examples, the secretion signal increases secretion of the fusion protein, or to be more precise, the protein of interest, from which the secretion signal has been cleaved off, from a eukaryotic host cell in comparison to a eukaryotic host cell expressing a fusion protein comprising a wild type Saccharomyces cerevisiae α-mating factor secretion signal (such as SEQ ID NO: 4) instead of the secretion signal described herein within the context of the invention. Thus, the MFα secretion signal (SEQ ID NO: 4) of wild type S. cerevisiae may be used as a control or reference for comparisons.

According to the present invention, due to (over)expression of the protein of interest as a part of the fusion protein encoded by the nucleic acid of the invention and optionally also the one or more component(s) of the SRP, the protein of interest (POI, after cleavage of the secretion signal and secretion itself) is obtainable in high yields, even when the biomass is kept low. Thus, a high specific yield, which is measured in mg POI/g dry biomass, may be in the range of 1 to 200, such as 50 to 200, such as 100-200, in the laboratory, pilot and industrial scale is feasible. “Increased secretion” as used herein relates to a higher amount of detectable protein of interest in the supernatant or culture medium of a host cell in comparison to a control; both cultivated under identical conditions (e.g., host cell species, culture medium, cultivation time, cultivation temperature, feeding and induction strategy). A control may be the same host cell, but where in the same host cell the protein of interest is expressed as a fusion protein comprising the MFα secretion signal such as depicted in SEQ ID NO: 4 instead of the secretion signal of the present invention. The increase may be expressed in fold change (FC) of secretion, e.g. an increase by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 5-fold or at least 10-fold. The amount of detectable protein of interest in the supernatant or culture medium of a host cell can be expressed as volumetric titer in [g of protein of interest/L of cell culture] or as yield in [mg protein of interest/g dry cell weight] or the like. The fold change of secretion is then the ratio of the volumetric titer or the yield of the cultivation of a host cell to the volumetric titer or the yield of the cultivation of the control.

The fusion protein, or the protein of interest, may further comprise one or more (detectable) tags, one or more protease cleavage sites and/or one or more linkers, such as between and connecting certain elements of the fusion protein (e.g., elements which are selected from the secretion signal, the signal peptide sequence, the α-mating factor (MFα) pro-sequence, or the protein of interest), and/or as part of any one of the elements, in particular as part of the protein of interest. For example, linkers may be positioned between the protein of interest, the tag(s) and/or the cleavage site(s). Thus, when the fusion protein of the present invention consists of from N-terminus to C-terminus a secretion signal as defined herein and of a protein of interest as also defined herein, such protein of interest may optionally further comprise one or more (detectable) tags, one or more protease cleavage sites and/or one or more linkers. In other words, one or more tags, one or more cleavage sites and/or one or more linkers as defined herein may also be fused N- or C-terminally to such protein of interest, thus being also comprised by said protein of interest, if the fusion protein of the invention consists of from N-terminus to C-terminus a secretion signal and of such protein of interest as described herein by the invention. In this context, such one or more tags, one or more cleavage sites and/or one or more linkers fused N- or C-terminally to such protein of interest may then be a part of such protein of interest. Also, when the fusion protein of the present invention consists of from N-terminus to C-terminus a secretion signal as defined herein and of a protein of interest as also defined herein, the secretion signal as defined herein may optionally further comprise one or more (detectable) tags, one or more a protease cleavage sites and/or one or more linkers. In this context, such one or more tags, one or more protease cleavage sites and/or one or more linkers fused to either N- or C-terminally to such secretion signal, or to the respective signal peptide sequence as defined herein or to the α-mating factor (MFα) pro-sequence as defined herein, may then be a part of such secretion signal.

Linker(s) are/is, e.g., defined in paragraph [38]. The (detectable) tags can be tags used for purification and/or to enhance the expression and/or solubility or the detection of the protein of interest. There are many purification, expression enhancing, solubility enhancing tags and tags that allow easy detection and quantification of the protein of interest known to a person skilled in the art. A “purification tag” (also called “affinity tag”) is an amino acid sequence that can be used for example for the purification of proteins where it is attached to (proteins of interest including affinity tag e.g. at its N-terminus). This tag has high affinity to appropriate ligands of a solid support, like chromatography resins or directly to the resins. By selectively binding of the protein of interest including the purification tag to the particular resin the protein of interest can be purified highly effective by only one chromatography step. Purification tags, are known to a person skilled in the art and may be a protein purification tag, preferably a GST tag, a FLAG tag, a polyarginine tag, a polyhistidine tag, such as a 6His-tag, an MBP tag, an S-tag, an influenza virus HA tag, a thioredoxin tag or a staphylococcal protein A tag. In more detail, a purification tag sequence used herein is any one of a histidine (His) tag, preferably a poly-histidine tag such as a hexahistidine (6 His) tag; an arginine-tag, preferably a poly-arginine tag, a peptide substrate for antibodies, a chitin binding domain, a RNAse S peptide, a protein A, S-galactosidase, a FLAG tag, a Strep II tag, a streptavidin binding peptide (SBP) tag, a calmodulin-binding peptide (CBP), a glutathione S-transferase (GST), a maltose-binding protein (MBP), a S-tag, a HA-tag, a c-Myc tag or any other tag known to be useful for the efficient purification of a protein it is fused to. Specifically, a fusion protein, in particular the protein of interest, including a poly-, or hexa-histidine tag (His-tag) can be captured and purified by IMAC, preferably using a Ni-NTA chromatography material. In one preferred embodiment of the present invention, a poly-, or hexa-histidine tag (His-tag), even more preferably a 6-His tag, comprised by the fusion protein, or comprised by the protein of interest as defined herein above, is applied herein. There are also many protease cleavage sites that can be used to cleave off the tags from the protein of interest after expression and/or secretion of the protein of interest from the host cell using the corresponding proteases known to a person skilled in the art. An “expression and/or solubility enhancing tag” can be fused C- or N-terminally to a protein of interest as defined herein. An expression and/or solubility enhancing tag can increase the expression and/or titer and/or solubility and/or soluble expression and/or soluble titer of the protein of interest when expressed in a host cell, e.g. a prokaryotic or a eukaryotic cell, a bacterial cell or yeast cell, e.g. E. coli, e.g. Pichia pastoris significantly, e.g. expressed in the cytosol, periplasma or secreted from the host cell compared to expression of the proteins of interest without the expression and/or solubility enhancing tag. An expression and/or solubility enhancing tag sequence used herein can be selected from the group consisting of a calmodulin-binding peptide (CBP), a poly Arg, a poly Lys, a G B1 domain, a protein D, a Z domain of Staphylococcal protein A, and a thioredoxin or any other tag known to improve the expression and/or solubility of the protein it is fused to e.g. during expression in a host cell. The expression and/or solubility enhancing tag can be based on highly charged peptides of bacteriophage genes, for example such as those listed in U.S. Pat. No. 8,535, 908 B2. Preferably, the solubility enhancing tag is selected from the group consisting of T7C, T7B, T7B1, T7B2, T7B3, T7B3, T7B4, T7B5, T7B6, T7B6, T7B7, T7B8, T7B9, T7B10, T7B11, T7B12, T7B13, T7A, T7A1, T7A2, T7A3, T7A4, T7A5, T7AC T3, N1, N2, N3, N4, N5, N6, N7, calmodulin-binding peptide (CBP), poly Arg, poly Lys, G B1 domain, protein D, Z domain of Staphylococcal protein A, DsbA, DsbC and thioredoxin and variants thereof gained by the substitution of a few amino acids such a 1, 2, 3 or even more. In one preferred embodiment of the present invention, a T7AC solubility tag comprised by the fusion protein, or comprised by the protein of interest as defined herein above, is applied herein. A “tag that allows easy detection and quantification of the protein of interest” is a tag that can be fused C- or N-terminally to a protein of interest as defined herein. It is a tag that can be used to detect, quantify, analyse the protein of interest throughout the production process (e.g. measurement of the titer of the protein of interest in fermentation broth or the content of the POI in different solutions throughout the production process, e.g. in chromatography eluates, cell homogenates, filter retentates or filtrates, etc. directly in-line, in-situ, on-line or atline by e.g. spectroscopic or fluorometric or other methods or offline by methods of the state of the art). Thereby, the tag can e.g. give the POI features like fluorescence, UV-VIS absorbance, as well as other absorbances useful for other spectroscopic or fluorometric methods that are used for on-line, in-line, atline or in-situ quantification methods known in the art. The tag can also be an affinity tag or other tag that can be used for quantitative affinity chromatography e.g. affinity HPLC or immunoassays like ELISA as an off-line measurement. E.g. the monitoring tag as one example of a tag that allows easy detection and quantification of the protein of interest is any one of m-Cherry, GFP or f-Actin or any other tag useful for detection or quantification of the protein of interest during production of the protein of interest including fermentation, isolation and purification by simple in-situ, inline online or atline detectors, like UV, IR, Raman, Fluorescence and the like. The detection tag as another example of a tag that allows easy detection and quantification of the protein of interest can also be a protein A Tag, S-galactosidase tag, FLAG tag, Strep tag or streptavidin binding peptide (SBP) tag or Strep Il-tag for the use in quantitative HPLC or ELISA.

In one embodiment of the present invention, the fusion protein comprising a secretion signal as defined herein (such as comprising a signal peptide sequence originating from a KRE1 protein or a signal peptide sequence originating from a SWP1 protein, optionally comprising an α-mating factor (MFα) pro-sequence) as well as comprising a protein of interest as defined herein, also comprises a solubility enhancing tag, even more preferably T7AC, and/or a purification tag, even more preferably a 6His tag, and/or a protease cleavage site for caspase, preferably for caspase-2 such as VDVAD, preferably wherein the solubility enhancing tag as defined herein and/or the purification tag as defined herein and/or the protease cleavage site as defined herein is/are fused N-terminally to the protein of interest as defined herein. In detail, in one embodiment of the invention, the fusion protein comprising a secretion signal comprising a signal peptide sequence originating from a KRE1 protein and an α-mating factor (MFα) pro-sequence as well as comprising a protein of interest as defined herein, also comprises a solubility enhancing tag, even more preferably T7AC, and/or a purification tag, even more preferably a 6His tag, and/or a protease cleavage site for caspase, preferably for caspase-2 such as VDVAD, preferably wherein the solubility enhancing tag as defined herein and/or the purification tag as defined herein and/or the protease cleavage site as defined herein is/are fused N-terminally to the protein of interest as defined herein. In detail, in another embodiment of the invention, the fusion protein comprising a secretion signal comprising a signal peptide sequence originating from a SWP1 protein and an α-mating factor (MFα) pro-sequence as well as comprising a protein of interest as defined herein, also comprises a solubility enhancing tag, even more preferably T7AC, and/or a purification tag, even more preferably a 6His tag, and/or a protease cleavage site for caspase, preferably for caspase-2 such as VDVAD, preferably wherein the solubility enhancing tag as defined herein and/or the purification tag as defined herein and/or the protease cleavage site as defined herein is/are fused N-terminally to the protein of interest as defined herein. In a preferred embodiment of the present invention, the fusion protein comprising a secretion signal as defined herein (such as comprising a signal peptide sequence originating from a KRE1 protein or a signal peptide sequence originating from a SWP1 protein and comprising an α-mating factor (MFα) pro-sequence) as well as comprising a protein of interest as defined herein, also comprises a solubility enhancing tag T7AC and a 6His purification tag and a protease cleavage site VDVAD, even more preferably wherein the solubility enhancing tag T7AC and the 6His purification tag and the protease cleavage site VDVAD are fused N-terminally to the protein of interest as defined herein. When the fusion protein of the present invention consists of from N-terminus to C-terminus a secretion signal as defined herein (such as comprising a signal peptide sequence originating from a KRE1 protein or a signal peptide sequence originating from a SWP1 protein, optionally comprising an α-mating factor (MFα) pro-sequence) and of a protein of interest as also defined herein, such protein of interest may optionally also comprise a solubility enhancing tag, even more preferably T7AC, and/or a purification tag, even more preferably a 6His tag, and/or a protease cleavage site for caspase, preferably for caspase-2 such as VDVAD, preferably wherein the solubility enhancing tag as defined herein and/or the purification tag as defined herein and/or the protease cleavage site as defined herein is/are fused N-terminally to the protein of interest as defined herein. In this context, such tag(s) and/or cleavage site is/are a part of the protein of interest. In a preferred embodiment, when the fusion protein of the present invention consists of from N-terminus to C-terminus a secretion signal comprising a signal peptide sequence originating from a KRE1 protein and an α-mating factor (MFα) pro-sequence and of a protein of interest as also defined herein, such protein of interest also comprises a solubility enhancing tag T7AC and a 6His purification tag and a protease cleavage site VDVAD, even more preferably wherein the solubility enhancing tag T7AC and the 6His purification tag and the protease cleavage site VDVAD are fused N-terminally to the protein of interest as defined herein. In another preferred embodiment, when the fusion protein of the present invention consists of from N-terminus to C-terminus a secretion signal comprising a signal peptide sequence originating from a SWP1 protein and an α-mating factor (MFα) pro-sequence and of a protein of interest as also defined herein, such protein of interest also comprises a solubility enhancing tag T7AC and a 6His purification tag and a protease cleavage site VDVAD, even more preferably wherein the solubility enhancing tag T7AC and the 6His purification tag and the protease cleavage site VDVAD are fused N-terminally to the protein of interest as defined herein. Again, in this context such T7AC and 6His tags and said VDVAD cleavage site are a part of the protein of interest.

### Secretion Signals

To be secreted, a protein has to travel through the intracellular secretory pathway of a cell that produces it. The protein is directed to this pathway, rather than to alternative cellular destinations, via an N-terminal secretion signal. At a minimum, a secretion signal comprises a signal peptide sequence. Signal peptide sequences typically consist of 13 to 36 mostly hydrophobic amino acids flanked by N-terminal basic amino acids and C-terminal polar amino acids. The signal peptide sequence can interact with the signal recognition particle (SRP) or other transport proteins (e.g., SND, GET) that mediates the co- or post-translational translocation of the nascent protein from the cytosol into the lumen of the ER. In the ER, the signal peptide sequence is typically cleaved off and the protein folds and undergoes post-translational modifications. The protein is then delivered from the ER to the Golgi apparatus and then on to secretory vesicles and the cell exterior. In addition to a signal peptide sequence, a subset of nascent proteins natively destined for secretion carry a secretion signal that also comprises a leader peptide such as the α-mating factor pro-sequence. Leader peptides typically consist of hydrophobic amino acids interrupted by charged or polar amino acids. Without wishing to be bound by theory, it is believed that the leader peptide slows down transport and ensures proper folding of the protein, and/or facilitates transport of the protein from the ER to the Golgi apparatus, where the leader peptide is typically cleaved off. “A signal peptide sequence originating from a KRE1 or a SWP1 protein” as used herein describes an amino acid sequence, i.e. the signal peptide sequence, which is present in the KRE1 protein as defined herein or the SWP1 protein as defined herein. Since the secretion signal including the signal peptide sequence is usually cleaved off during secretion, signal peptide sequences described herein originate from the KRE1 protein or the SWP1 before secretion and/or before cleavage of the signal peptide sequence. “Originating from” may be used interchangeably with “derived from”.

KRE1, also known as Killer toxin-resistance protein 1, is a protein of yeast that is secreted. KRE1 may be involved in a late stage of cell wall 1,6-beta-glucan synthesis and assembly. It has a structural, rather than enzymatic, function within cell wall 1,6-beta-glucan assembly and architecture, possibly by being involved in covalently cross-linking 1,6-beta-glucans to other cell wall components such as 1,3-beta-glucan, chitin and certain mannoproteins. It furthermore acts as the plasma membrane receptor for the yeast K1 viral toxin. As such, it carries a signal peptide sequence. KRE1 might be from any eukaryotic species, in particular from any yeast. Exemplary yeasts include, but are not limited to, Komagataella phaffii (Pichia pastoris), Hansenula polymorpha, Saccharomyces cerevisiae, Saccharomyces paradoxus, Saccharomyces eubayanus, Saccharomyces kudriavzevii, Saccharomyces kluyveri, Saccharomyces uvarum, Kluyveromyces lactis, Yarrowia lipolytica, Pichia methanolica, Candida boidinii, Komagataella spp. and Schizosaccharomyces pombe. KRE1 can also be from Trichoderma reesei or Aspergillus niger. Preferably, the KRE1 is from K. phaffii. The signal peptide sequence originating from KRE1 may comprise or consist of the first 18 amino acids or a functional homolog thereof of a full-length KRE1 protein (id est the protein including the signal peptide translated from the mRNA encoding the KRE1 protein) such as the KRE1 protein from K. phaffii. In a preferred embodiment, the KRE1 protein corresponds to UniProt database entry F2QWV3, sequence version 1 of 31 May 2011 (chromosomal location PP7435_Chr3-0933) or a functional homolog thereof, wherein the signal peptide sequence preferably corresponds to amino acids 1-18 of said database entry depicted also in SEQ ID NO: 1. Accordingly, the signal peptide sequence originating from a KRE1 protein may comprise or consist of SEQ ID NO: 1 or a functional homolog thereof. The signal peptide sequence originating from a KRE1 protein may consist of SEQ ID NO: 1 or a functional homolog thereof. The signal peptide sequence originating from a KRE1 protein may comprise at least any one of 80%, 85%, 90%, 94% or 95% sequence identity to SEQ ID NO: 1, which may refer to a functional homolog thereof as defined herein. The signal peptide sequence originating from a KRE1 protein may comprise at least 90% sequence identity to SEQ ID NO: 1. The signal peptide sequence originating from a KRE1 protein may comprise at least 94% sequence identity to SEQ ID NO: 1. The signal peptide sequence originating from a KRE1 protein may comprise at least 95% sequence identity to SEQ ID NO: 1.

SWP1, also known as Dolichyl-diphosphooligosaccharide--protein glycosyltransferase subunit SWP1, is a subunit of the oligosaccharyl transferase (OST) complex that catalyzes the initial transfer of a defined glycan (GlcMan₉GlcNAc₂ in eukaryotes) from the lipid carrier dolichol-pyrophosphate to an asparagine residue within an Asn-X-Ser/Thr consensus motif in nascent polypeptide chains, the first step in protein N-glycosylation. Also SWP1 carries a signal peptide sequence. SWP1 might be from any eukaryotic species, in particular from any yeast. Exemplary yeasts include, but are not limited to, Komagataella phaffii (Pichia pastoris), Hansenula polymorpha, Saccharomyces cerevisiae, Saccharomyces paradoxus, Saccharomyces eubayanus, Saccharomyces kudriavzevii, Saccharomyces kluyveri, Saccharomyces uvarum, Kluyveromyces lactis, Yarrowia lipolytica, Pichia methanolica, Candida boidinii, Komagataella spp., Komagataella pastoris and Schizosaccharomyces pombe. SWP1 can also be from Trichoderma reesei or Aspergillus niger. Preferably, the SWP1 is from K. phaffii. The signal peptide sequence originating from SWP1 may comprise or consist of the first 18 amino acids of a full-length SWP1 (id est the protein including the signal peptide translated from the mRNA encoding the SWP1 protein) protein such as the SWP1 protein from K. phaffii or a functional homolog thereof. In a preferred embodiment, the SWP1 protein corresponds to UniProt database entry F2QN13, sequence version 1 of 31 May 2011 (gene PP7435_Chr1-0255) or a functional homolog thereof, wherein the signal peptide sequence preferably corresponds to amino acids 1-18 of said database entry depicted also in SEQ ID NO: 2. Accordingly, the signal peptide sequence originating from a SWP1 protein may comprise or consist of SEQ ID NO: 2 or a functional homolog thereof. The signal peptide sequence originating from a SWP1 protein may consist of SEQ ID NO: 2 or a functional homolog thereof. The signal peptide sequence originating from a SWP1 protein may comprise at least any one of 80%, 85%, 90%, 94% or 95% sequence identity to SEQ ID NO: 2, which may refer to a functional homolog thereof as defined herein. The signal peptide sequence originating from a SWP1 protein may comprise at least 90% sequence identity to SEQ ID NO: 2. The signal peptide sequence originating from a SWP1 protein may comprise at least 94% sequence identity to SEQ ID NO: 2. The signal peptide sequence originating from a SWP1 protein may comprise at least 95% sequence identity to SEQ ID NO: 2. Preferably, the SWP1 is from K. pastoris. Accordingly, the signal peptide sequence originating from a SWP1 protein may comprise SEQ ID NO: 52 or a functional homolog thereof. The signal peptide sequence originating from a SWP1 protein may consist of SEQ ID NO: 52 or a functional homolog thereof. The signal peptide sequence originating from a SWP1 protein may comprise at least any one of 80%, 85%, 90%, 94% or 95% sequence identity to SEQ ID NO: 52, which may refer to a functional homolog thereof as defined herein. The signal peptide sequence originating from a SWP1 protein may comprise at least 90% sequence identity to SEQ ID NO: 52. The signal peptide sequence originating from a SWP1 protein may comprise at least 95% sequence identity to SEQ ID NO: 52.

α-Mating factor (MFα), also known as Mating factor alpha-1, alpha-1 mating pheromone or mating factor alpha, is a hormone, wherein the active factor (MFα without secretion signal) is excreted into the culture medium by haploid cells of the alpha mating type and acts on cells of the opposite mating type (type a). It mediates the conjugation process between the two types by inhibiting the initiation of DNA synthesis in type a cells and synchronizing them with type alpha. MFα carries a secretion signal comprising a signal peptide sequence (pre-sequence) and a pro-sequence. MFα might be from any eukaryotic species, in particular from any yeast, preferably from any yeast of the Saccharomyces genus such as Saccharomyces paradoxus, Saccharomyces eubayanus, Saccharomyces cerevisiae, Saccharomyces kluyveri, Saccharomyces uvarum or Saccharomyces kudriavzevii. Exemplary yeasts include, but are not limited to, Komagataella phaffii (Pichia pastoris), Hansenula polymorpha, Saccharomyces cerevisiae, Saccharomyces paradoxus, Saccharomyces eubayanus, Saccharomyces kudriavzevii, Saccharomyces kluyveri, Saccharomyces uvarum, Kluyveromyces lactis, Yarrowia lipolytica, Pichia methanolica, Candida boidinii, Komagataella spp. and Schizosaccharomyces pombe. MFα can also be from Trichoderma reesei or Aspergillus niger. Preferably, the origin is from S. cerevisiae. The pro-sequence may comprise or consist of the amino acids 20-89 of the full-length MFα protein (id est the protein including the signal peptide and the pro-sequence translated from the mRNA encoding the MFα protein) such as the MFα protein from S. cerevisiae or a functional homolog thereof. In a preferred embodiment, the full-length MFα protein corresponds to UniProt database entry P01149, sequence version 1 of 1 Apr. 1988 or a functional homolog thereof, wherein the α-mating factor (MFα) pro-sequence preferably corresponds to amino acids 20-89, more preferably amino acids 20-85 as depicted in SEQ ID NO: 53, of said database entry. The MFα pro-sequence may comprise SEQ ID NO: 3 or a functional homolog thereof. The MFα pro-sequence may consist of SEQ ID NO: 3 or a functional homolog thereof. The MFα pro-sequence may comprise at least any one of 80%, 85%, 90%, 95% or 98% sequence identity to SEQ ID NO: 3, which may refer to a functional homolog thereof as defined herein. The MFα pro-sequence may comprise at least 90% sequence identity to SEQ ID NO: 3. The MFα pro-sequence may comprise at least 95% sequence identity to SEQ ID NO: 3. The MFα pro-sequence may comprise at least 98% sequence identity to SEQ ID NO: 3. The MFα pro-sequence may comprise SEQ ID NO: 53 or a functional homolog thereof. The MFα pro-sequence may consist of SEQ ID NO: 53 or a functional homolog thereof. The MFα pro-sequence may comprise at least any one of 80%, 85%, 90%, 95% or 98% sequence identity to SEQ ID NO: 53, which may refer to a functional homolog thereof as defined herein. The MFα pro-sequence may comprise at least 90% sequence identity to SEQ ID NO: 53. The MFα pro-sequence may comprise at least 95% sequence identity to SEQ ID NO: 53. The MFα pro-sequence may comprise at least 98% sequence identity to SEQ ID NO: 53.

The MFα pro-sequence may originate from Saccharomyces paradoxus. The MFα pro-sequence may comprise SEQ ID NO: 74 or a functional homolog thereof. The MFα pro-sequence may consist of SEQ ID NO: 74 or a functional homolog thereof. The MFα pro-sequence may comprise at least any one of 80%, 85%, 90%, 95% or 98% sequence identity to SEQ ID NO: 74, which may refer to a functional homolog thereof as defined herein. The MFα pro-sequence may comprise at least 90% sequence identity to SEQ ID NO: 74. The MFα pro-sequence may comprise at least 95% sequence identity to SEQ ID NO: 74. The MFα pro-sequence may comprise at least 98% sequence identity to SEQ ID NO: 74. The MFα pro-sequence may comprise SEQ ID NO: 75 or a functional homolog thereof. The MFα pro-sequence may consist of SEQ ID NO: 75 or a functional homolog thereof. The MFα pro-sequence may comprise at least any one of 80%, 85%, 90%, 95% or 98% sequence identity to SEQ ID NO: 75, which may refer to a functional homolog thereof as defined herein. The MFα pro-sequence may comprise at least 90% sequence identity to SEQ ID NO: 75. The MFα pro-sequence may comprise at least 95% sequence identity to SEQ ID NO: 75. The MFα pro-sequence may comprise at least 98% sequence identity to SEQ ID NO: 75. The MFα pro-sequence may comprise SEQ ID NO: 76 or a functional homolog thereof. The MFα pro-sequence may consist of SEQ ID NO: 76 or a functional homolog thereof. The MFα pro-sequence may comprise at least any one of 80%, 85%, 90%, 95% or 98% sequence identity to SEQ ID NO: 76, which may refer to a functional homolog thereof as defined herein. The MFα pro-sequence may comprise at least 90% sequence identity to SEQ ID NO: 76. The MFα pro-sequence may comprise at least 95% sequence identity to SEQ ID NO: 76. The MFα pro-sequence may comprise at least 98% sequence identity to SEQ ID NO: 76. The MFα pro-sequence may comprise SEQ ID NO: 77 or a functional homolog thereof. The MFα pro-sequence may consist of SEQ ID NO: 77 or a functional homolog thereof. The MFα pro-sequence may comprise at least any one of 80%, 85%, 90%, 95% or 98% sequence identity to SEQ ID NO: 77, which may refer to a functional homolog thereof as defined herein. The MFα pro-sequence may comprise at least 90% sequence identity to SEQ ID NO: 77. The MFα pro-sequence may comprise at least 95% sequence identity to SEQ ID NO: 77. The MFα pro-sequence may comprise at least 98% sequence identity to SEQ ID NO: 77. The MFα pro-sequence may comprise SEQ ID NO: 78 or a functional homolog thereof. The MFα pro-sequence may consist of SEQ ID NO: 78 or a functional homolog thereof. The MFα pro-sequence may comprise at least any one of 80%, 85%, 90%, 95% or 98% sequence identity to SEQ ID NO: 78, which may refer to a functional homolog thereof as defined herein. The MFα pro-sequence may comprise at least 90% sequence identity to SEQ ID NO: 78. The MFα pro-sequence may comprise at least 95% sequence identity to SEQ ID NO: 78. The MFα pro-sequence may comprise at least 98% sequence identity to SEQ ID NO: 78.

The MFα pro-sequence may originate from Saccharomyces eubayanus. The MFα pro-sequence may comprise SEQ ID NO: 79 or a functional homolog thereof. The MFα pro-sequence may consist of SEQ ID NO: 79 or a functional homolog thereof. The MFα pro-sequence may comprise at least any one of 80%, 85%, 90%, 95% or 98% sequence identity to SEQ ID NO: 79, which may refer to a functional homolog thereof as defined herein. The MFα pro-sequence may comprise at least 90% sequence identity to SEQ ID NO: 79. The MFα pro-sequence may comprise at least 95% sequence identity to SEQ ID NO: 79. The MFα pro-sequence may comprise at least 98% sequence identity to SEQ ID NO: 79.

The MFα pro-sequence may originate from Saccharomyces kudriavzevii. The MFα pro-sequence may comprise SEQ ID NO: 80 or a functional homolog thereof. The MFα pro-sequence may consist of SEQ ID NO: 80 or a functional homolog thereof. The MFα pro-sequence may comprise at least any one of 80%, 85%, 90%, 95% or 98% sequence identity to SEQ ID NO: 80, which may refer to a functional homolog thereof as defined herein. The MFα pro-sequence may comprise at least 90% sequence identity to SEQ ID NO: 80. The MFα pro-sequence may comprise at least 95% sequence identity to SEQ ID NO: 80. The MFα pro-sequence may comprise at least 98% sequence identity to SEQ ID NO: 80.

The MFα pro-sequence preferably has a Ser at a position corresponding to position 23 of SEQ ID NO: 53 and/or Glu at a position corresponding to position 64 of SEQ ID NO: 53, preferably at both positions. This can further increase secretion. SEQ ID NO: 3 already contains these mutations.

A functional homolog is a functional equivalent of a nucleic acid sequence or a peptide, polypeptide or protein described in this document. A functional homolog may be a biologically active sequence that has at least about 70%, at least about 80%, at least about 90% or at least about 95% amino acid sequence identity with a given sequence of a polypeptide, such as the sequence of SEQ ID NO: 1, 2, 3, 52, or 53. In some embodiments a functional homolog is a biologically active sequence that has at least about 70%, at least about 80%, at least about 90% or at least about 95% amino acid sequence identity with the native sequence polypeptide. With regard to nucleic acid sequences, the degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the nucleic acid sequences described herein could be synthesized to give a nucleic acid sequence significantly different from that shown in their indicated sequence. The encoded amino acid sequence thereof would, however, be preserved.

The functional homolog may also describe a functional equivalent of an amino acid sequence or a peptide, polypeptide or protein described in this document, which has up to 5 conservative mutations, i.e. the functional homolog can have 1, 2, 3, 4 or 5 conservative mutations. In some embodiments, in particular for functional homologs of the MFα pro-sequence, the functional homolog can also have 1, 2, 3, 4, 5, 6, 7, 8, 9, 10, 11, 12, 13 or 14 conservative mutations. A “conservative mutation” as used herein is preferably a mutation that results in a point mutation which is e.g., a substitution, insertion or deletion of one amino acid, in particular where the substitution is the replacement of an amino acid residue with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. A conservative mutation can also be any a substitution, insertion or deletion of one amino acid that does not influence the biological activity of the amino acid sequence of a peptide, polypeptide or protein described herein. A functional homolog may have up to 14 conservative mutations. A functional homolog may have up to 13 conservative mutations. A functional homolog may have up to 12 conservative mutations. A functional homolog may have up to 11 conservative mutations. A functional homolog may have 10 conservative mutations. A functional homolog may have up to 9 conservative mutations A functional homolog may have up to 8 conservative mutations. A functional homolog may have up to 7 conservative mutations. A functional homolog may have 6 conservative mutations. A functional homolog may have up to 5 conservative mutations. A functional homolog may have up to 4 conservative mutations. A functional homolog may have up to 3 conservative mutations. A functional homolog may have up to 2 conservative mutations. A functional homolog may have 1 conservative mutation.

TABLE 1
Overview of parts of secretion signals.

| Signal | Nucleotide Sequence (5′-3′) | Amino Acid Sequence |
|---|---|---|
| SP4 (signal peptide | ATGAAGCTGATCTCCGTGGGTA | MKLISVGIVTTLLTLASC |
| sequence of SWP1 of K. | TAGTGACGACATTACTGACTTT | (SEQ ID NO: 2) |
| phaffii) | GGCCAGTTGC (SEQ ID NO: 18) | |
| signal peptide sequence | | MKLFFVGIVTTLLTLVSC |
| of SWP1 of K. pastoris | | (SEQ ID NO: 52) |
| SP14 (signal peptide | ATGTTAAACAAGCTGTTCATTG | MLNKLFIAILIVITAVIG (SEQ |
| sequence of KRE1 of K. | CAATACTCATAGTCATCACTGC | ID NO: 1) |
| phaffii) | TGTCATAGGC (SEQ ID NO: 19) | |
| WT MFα pro-sequence of | | APVNTTTEDETAQIPAEAVIG |
| Saccharomyces | | YLDLEGDFDVAVLPFSNSTN |
| cerevisiae | | NGLLFINTTIASIAAKEEGVSL |
| | | DKR (SEQ ID NO: 53) |
| MFαpro-sequence based | GCCCCTGTTAACACTACCACTG | APVNTTTEDETAQIPAEAVIG |
| on WT MF-alpha pro | AAGACGAGACTGCTCAAATTCC | YSDLEGDFDVAVLPFSNSTN |
| sequence of | AGCTGAAGCAGTTATCGGTTAC | NGLLFINTTIASIAAKEEGVSL |
| Saccharomyces | TCTGACCTTGAGGGTGATTTCG | EKR (SEQ ID NO: 3) |
| cerevisiae including two | ACGTCGCTGTTTTGCCTTTCTC | |
| mutations: L23S, D64E | TAACTCCACTAACAACGGTTTG | |
| | TTGTTCATTAACACCACTATCGC | |
| | TTCCATTGCTGCTAAGGAAGAG | |
| | GGTGTCTCTCTCGAGAAGAGA | |
| | (SEQ ID NO: 20) | |
| MFα secretion signal | ATGAGATTCCCATCTATTTTCAC | MRFPSIFTAVLFAASSALAAP |
| (pre-pro-sequence) that | CGCTGTCTTGTTCGCTGCCTCC | VNTTTEDETAQIPAEAVIGYS |
| can be used as reference | TCTGCATTGGCTGCCCCTGTTA | DLEGDFDVAVLPFSNSTNNG |
| for comparisons based on | ACACTACCACTGAAGACGAGAC | LLFINTTIASIAAKEEGVSLEK |
| WT MFα secretion signal | TGCTCAAATTCCAGCTGAAGCA | R (SEQ ID NO: 4) |
| of Saccharomyces | GTTATCGGTTACTCTGACCTTG | |
| cerevisiae including two | AGGGTGATTTCGACGTCGCTGT | |
| mutations: L23S, D64E | TTTGCCTTTCTCTAACTCCACTA | |
| | ACAACGGTTTGTTGTTCATTAAC | |
| | ACCACTATCGCTTCCATTGCTG | |
| | CTAAGGAAGAGGGTGTCTCTCT | |
| | CGAGAAGAGA (SEQ ID NO: 21) | |

The present invention further relates to a secretion signal as defined herein. Further disclosed herein is a secretion signal, the secretion signal comprising (i) a signal peptide sequence originating from a KRE1 protein; and (ii) an α-mating factor (MFα) pro-sequence. The signal peptide sequence may comprise SEQ ID NO: 1 or a functional homolog thereof and the α-mating factor (MFα) pro-sequence may comprise SEQ ID NO: 3 or a functional homolog thereof. The signal peptide sequence may comprise SEQ ID NO: 1 or a functional homolog thereof and the α-mating factor (MFα) pro-sequence may comprise SEQ ID NO: 53 or a functional homolog thereof. The present invention further relates to a secretion signal, the secretion signal comprising (i) a signal peptide sequence originating from a SWP1 protein; and (ii) an α-mating factor (MFα) pro-sequence. The signal peptide sequence may comprise SEQ ID NO: 2 or a functional homolog thereof and the α-mating factor (MFα) pro-sequence may comprise SEQ ID NO: 3 or a functional homolog thereof. The signal peptide sequence may comprise SEQ ID NO: 2 or a functional homolog thereof and the α-mating factor (MFα) pro-sequence may comprise SEQ ID NO: 53 or a functional homolog thereof. The signal peptide sequence may comprise SEQ ID NO: 52 or a functional homolog thereof and the α-mating factor (MFα) pro-sequence may comprise SEQ ID NO: 3 or a functional homolog thereof. The signal peptide sequence may comprise SEQ ID NO: 52 or a functional homolog thereof and the α-mating factor (MFα) pro-sequence may comprise SEQ ID NO: 53 or a functional homolog thereof.

### Proteins of Interest

The term “protein of interest” (POI) as used herein refers to a protein that is produced by means of recombinant technology in a host cell. More specifically, the protein may either be a polypeptide not naturally occurring in the host cell, i.e. a heterologous protein e.g. an artificial protein such as a protein not naturally produced by wild-type cells, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of the promoter sequence. In general, the proteins of interest referred to herein may be produced by methods of recombinant expression well known to a person skilled in the art. The protein of interest may be a recombinant protein.

There is no limitation with respect to the protein of interest (POI). The POI is usually a eukaryotic or prokaryotic polypeptide, variant or derivative thereof, or an artificial polypeptide, such as a polypeptide not naturally produced by wild-type cells. The POI can be any eukaryotic or prokaryotic protein. The protein can be a naturally secreted protein or an intracellular protein, i.e. a protein which is not naturally secreted. The present invention also includes biologically active fragments of proteins. In another embodiment, a POI may be an amino acid chain or present in a complex, such as a dimer, trimer, hetero-dimer, multimer or oligomer. Fusion of the POI with the secretion signal of the invention can render any POI to be secreted. The POI may be a protein that requires co-translational translocation.

The protein of interest may be a protein used as nutritional, dietary, digestive, supplements, such as in food products, feed products, or cosmetic products. The food products may be, for example, bouillon, desserts, cereal bars, confectionery, sports drinks, dietary products or other nutrition products. Preferably, the protein of interest is a food additive.

In another embodiment, the protein of interest may be used in animal feeds.

Further examples of POI include anti-microbial proteins, such as lactoferrin, lysozyme, lactoferricin, lactohedrin, kappa-casein, haptocorrin, lactoperoxidase, a milk protein, acute-phase proteins, e.g., proteins that are produced normally in production animals in response to infection, and small anti-microbial proteins such as lysozyme and lactoferrin. Other examples include bactericidal protein, antiviral proteins, acute phase proteins (induced in production animals in response to infection), probiotic proteins, bacteriostatic protein, and cationic antimicrobial proteins.

“Feed” means any natural or artificial diet, meal or the like or components of such meals intended or suitable for being eaten, taken in, digested, by a non-human animal. A “feed additive” generally refers to substances added in a feed. It typically includes one or more compounds such as vitamins, minerals, enzymes and suitable carriers and/or excipient. For the present invention, a food additive may be an enzyme or other proteins. Examples of enzymes which can be used as feed additive include phytase, xylanase and β-glucanase. A “food” means any natural or artificial diet meal or the like or components of such meals intended or suitable for being eaten, taken in, digested, by a human being.

A “food additive” is generally refers to substances added in a food. It typically includes one or more compounds such as vitamins, minerals, enzymes and suitable carriers and/or excipient. For the present invention, a food additive may be an enzyme or other proteins. Examples of enzymes which can be used as food additive include protease, lipase, lactase, pectin methyl esterase, pectinase, transglutaminase, amylase, β-glucanase, acetolactate decarboxylase and laccase.

In some embodiments, the food additive is an anti-microbial protein, which includes, for example, (i) anti-microbial milk proteins (either human or non-human) lactoferrin, lysozyme, lactoferricin, lactohedrin, kappa-casein, haptocorrin, lactoperoxidase, alpha-1-antitrypsin, and immunoglobulins, e.g., IgA, (ii) acute-phase proteins, such as C-reactive protein (CRP); lactoferrin; lysozyme; serum amyloid A (SAA); ferritin; haptoglobin (Hp); complements 2-9, in particular complement-3; seromucoid; ceruloplasmin (Cp); 15-keto-13,14-dihydro- prostaglandin F2 alpha (PGFM); fibrinogen (Fb); alpha(1)-acid glycoprotein (AGP); alpha(1)-antitrypsin; mannose binding protein; lipoplysaccharide binding protein; alpha-2 macroglobulin and various defensins, (iii) antimicrobial peptides, such as cecropin, magainin, defensins, tachyplesin, parasin I.buforin I, PMAP-23, moronecidin, anoplin, gambicin, and SAMP-29, and (iv) other anti-microbial protein(s), including CAP37, granulysin, secretory leukocyte protease inhibitor, CAP18, ubiquicidin, bovine antimicrobial protein-1, Ace-AMP1, tachyplesin, big defensin, Ac-AMP2, Ah-AMP1, and CAP18.

A POI may be an enzyme. Preferred enzymes are those which can be used for industrial application, such as in the manufacturing of a detergent, starch, fuel, textile, pulp and paper, oil, personal care products, or such as for baking, organic synthesis, and the like. Examples of such enzymes include protease, amylase, lipase, mannanase and cellulose for stain removal and cleaning; pullulanase amylase and amyloglucosidase for starch liquefaction and saccharification; glucose isomerase for glucose to fructose conversion; cyclodextrin-glycosyltransferase for cyclodextrin production; xylanase for xiscosity reduction in fuel and starch; amylase, xylanase, lipase, phospholipase, glucose, oxidase, lipoxygenase, transglutaminase for dough stability and conditioning in baking; cellulase in textile manufacturing for denim finishing and cotton softening; amylase for de-sizing of texile; pectate lyase for scouring; catalase for bleach termination; laccase for bleaching; peroxidase for excess dye removal; lipase, protease, amylase, xylanase, cellulose, in pulp and paper production; lipase for transesterification and phospholipase for de-gumming in fat processing fats and oils; lipase for resolution of chiral alcohols and amides in organic synthesis; acylase for synthesis of semisynthetic penicillin, nitrilase for the synthesis of enantiopure carboxylic acids; protease and lipase for leather production; amyloglucosidase, glucose oxidase, and peroxidase for the making personal care products (see Kirk et al., Current Opinion in Biotechnology (2002) 13: 345-351).

A POI may be a therapeutic protein. A POI may be but is not limited to a protein suitable as a biopharmaceutical substance like an antibody or antibody fragment, growth factor, hormone, enzyme, or vaccine.

The POI may be a naturally secreted protein or an intracellular protein, i.e. a protein which is not naturally secreted. The present invention also provides for the recombinant production of functional homologues, functional equivalent variants, derivatives and biologically active fragments of naturally secreted or not naturally secreted proteins. Functional homologues are preferably identical with or correspond to and have the functional characteristics of a sequence.

The POI may be structurally similar to the native protein and may be originating from the native protein by addition of one or more amino acids to either or both the C- and N-terminal end or the side-chain of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Such modifications are well known for several of the proteins mentioned above.

Preferably, the protein of interest is a mammalian polypeptide or even more preferably a human polypeptide. Preferably, the protein of interest is a therapeutic or biopharmaceutical protein. Especially preferred therapeutic proteins, which refer to any polypeptide, protein, protein variant, fusion protein and/or fragment thereof which may be administered to a mammal even more preferred to a human. The protein of interest can also be an artificial protein, or a part of a natural protein or of natural proteins or of artificial proteins or a fusion protein. It is envisioned but not required that therapeutic protein according to the present invention is heterologous to the cell. Examples of proteins that can be produced by the cell of the present invention are, without limitation, enzymes, regulatory proteins, receptors, peptide hormones, growth factors, cytokines, scaffold binding proteins (e.g. a mutein based on the lipocalin family), structural proteins, lymphokines, adhesion molecules, receptors, membrane or transport proteins, and any other polypeptides that can serve as agonists or antagonists and/or have therapeutic or diagnostic use. Moreover, the proteins of interest may be antigens as used for vaccination, vaccines, antigen-binding proteins, immune stimulatory proteins. It may also be an antigen-binding fragment of an antibody, which can include any suitable antigen-binding antibody fragment known in the art. For example, an antibody fragment may include but not limited to Fv (a molecule comprising the VL and VH), single-chain Fv (scFV) (a molecule comprising the VL and VH connected with by peptide linker), Fab, Fab', F(ab′)₂, single domain antibody (sdAb) (molecules comprising a single variable domain and 3 CDR), and multivalent presentations thereof. The antibody or fragments thereof may be murine, human, humanized or chimeric antibody or fragments thereof. Examples of therapeutic proteins include an antibody, polyclonal antibody, monoclonal antibody, recombinant antibody, antibody fragments, such as Fab′, F(ab′)₂, Fv, scFv, di-scFvs, bi-scFvs, tandem scFvs, bispecific tandem scFvs, sdAb, VHH, V_{H}, and V_{L}, or human antibody, humanized antibody, chimeric antibody, IgA antibody, IgD antibody, IgE antibody, IgG antibody, IgM antibody, intrabody, minibody or synthetic binding proteins constructed using a fibronectin type III domain (FN3) as a molecular scaffold also known as monobody.

The protein of interest may further be selected from the group consisting of an antibody such as a chimeric, humanized or human antibody, or a bispecific antibody, or an antigen-binding antibody fragment such as Fab or F(ab)₂, single chain antibodies such as scFv, single domain antibodies such as VHH fragments of camelid or heavy chain antibodies or domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.

Therapeutic proteins include, but are not limited to, insulin, insulin-like growth factor, hGH, tPA, cytokines, e.g. interleukins such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF) TNF alpha and TNF beta, TRAIL; G-CSF, GM-CSF, M-CSF, MCP-1 and VEGF.

In a preferred embodiment, the protein is an antibody. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” Numerous antibody coding sequences have been described; and others may be raised by methods well-known in the art.

For example, antibodies or antigen binding antibody fragments may be produced by methods known in the art. Generally, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc.). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions (VK and VH), obtained from antibody producing cells of one species with the constant light and heavy chain regions from another. Typically, chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659.

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287.

In addition to entire immunoglobulins (or their recombinant counterparts), immunoglobulin fragments comprising the epitope binding site (e.g., Fab′, F(ab′)2, or other fragments) may be synthesized. “Fragment” or minimal immunoglobulins may be designed utilizing recombinant immunoglobulin techniques. For instance “Fv” immunoglobulins for use in the present invention may be produced by synthesizing a variable light chain region and a variable heavy chain region. Combinations of antibodies are also of interest, e.g. diabodies, which comprise two distinct Fv specificities.

Immunoglobulins may be modified post-translationally, e.g. to add chemical linkers, detectable moieties, such as fluorescent dyes, enzymes, substrates, chemiluminescent moieties and the like, or specific binding moieties, such as streptavidin, Avidin, or biotin, and the like may be utilized in the methods and compositions of the present invention.

Further examples of therapeutic proteins include blood coagulation factors (VII, VIII, IX), alkaline protease from Fusarium, calcitonin, CD4 receptor darbepoetin, DNase (cystic fibrosis), erythropoetin, eutropin (human growth hormone derivative), follicle stimulating hormone (follitropin), gelatin, glucagon, glucocerebrosidase (Gaucher disease), glucosamylase from A. niger, glucose oxidase from A. niger, gonadotropin, growth factors (GCSF, GMCSF), growth hormones (somatotropines),hepatitis B vaccine, hirudin, human antibody fragment, human apolipoprotein Al, human calcitonin precursor ,human collagenase IV, human epidermal growth factor, human insulin-like growth factor, human interleukin 6, human laminin, human proapolipoprotein Al, human serum albumin, insulin and muteins, insulin, interferon alpha and muteins, interferon beta, interferon gamma (mutein), interleukin 2, luteinization hormone, monoclonal antibody 5T4, mouse collagen, OP-1 (osteogenic, neuroprotective factor), oprelvekin (interleukin 11-agonist), organophosphohydrolase, PDGF-agonist, phytase, platelet derived growth factor (PDGF), recombinant plasminogen-activator G, staphylokinase, stem cell factor, tetanus toxin fragment C, tissue plasminogen-activator, and tumor necrosis factor (see Schmidt, Appl Microbiol Biotechnol (2004) 65: 363-372).

The protein of interest may comprise or consist of the amino acid sequence depicted in SEQ ID NO: 26. The protein of interest may comprise or consist of the amino acid sequence depicted in SEQ ID NO: 27. The protein of interest may comprise or consist of the amino acid sequence depicted in SEQ ID NO: 28. The protein of interest may comprise or consist of the amino acid sequence depicted in SEQ ID NO: 29. When the fusion protein of the present invention consists of from N-terminus to C-terminus a secretion signal as defined herein and of a protein of interest as depicted in SEQ ID NO: 26, 27, 28 or 29, such protein of interest may optionally further comprise one or more (detectable) tags, one or more protease cleavage sites and/or one or more linkers as defined elsewhere herein. In other words, one more tags, one or more cleavage sites and/or one or more linkers as defined herein may also be fused N- or C-terminally to such protein of interest as depicted in SEQ ID NO: 26, 27, 28 or 29, thus being also comprised by said protein of interest, if the fusion protein of the invention consists of from N-terminus to C-terminus a secretion signal and such protein of interest as defined in this context. In this context, such one or more tags, one or more cleavage sites and/or one or more linkers fused N- or C-terminally to such protein of interest as defined elsewhere herein may then be a part of such protein of interest. Also, when the fusion protein of the present invention consists of from N-terminus to C-terminus a secretion signal as defined herein and of a protein of interest as depicted in SEQ ID NO: 26, 27, 28 or 29, the secretion signal as defined herein may optionally further comprise one or more (detectable) tags, one or more a protease cleavage sites and/or one or more linkers as it has already been defined herein.

TABLE 2
Sequences of exemplary proteins to be expressed and secreted.

| Gene of Interest | Coding DNA sequence (5′-3′) | Protein sequence (* Stop codon) |
|---|---|---|
| VHH (can be expressed | CAGGTTCAGCTGCAGGAGT | QVQLQESGGGLVQAGGSLRLS |
| under pAOX1, and CYC1 | CCGGTGGTGGTCTGGTTCA | CAASGRTFTSFAMGWFRQAPG |
| can be used as the | AGCCGGTGGTTCATTAAGA | KEREFVASISRSGTLTRYADSA |
| terminator), (Prielhofer et | TTGTCCTGTGCTGCCTCTG | KGRFTISVDNAKNTVSLQMDNL |
| al. 2017) | GTAGAACTTTCACTTCTTTC | NPDDTAVYYCAADLHRPYGPG |
| | GCAATGGGTTGGTTTAGAC | TQRSDEYDSWGQGTQVTVSS |
| | AAGCACCTGGAAAAGAGAG | GGGSGGGGSGGGGSGGGGS |
| | AGAGTTTGTTGCTTCTATCT | GGGGSGGGEVQLVESGGALV |
| | CCAGATCCGGTACTTTAAC | QPGGSLRLSCAASGFPVNRYS |
| | TAGATACGCTGACTCTGCC | MRWYRQAPGKEREWVAGMSS |
| | AAGGGTAGATTCACTATTT | AGDRSSYEDSVKGRFTISRDDA |
| | CTGTTGACAACGCCAAGAA | RNTVYLQMNSLKPEDTAVYYC |
| | CACTGTTTCTTTGCAAATG | NVNVGFEYWGQGTQVTVSSG |
| | GACAACCTTAACCCAGATG | GHHHHHH** (SEQ ID NO: 26) |
| | ACACCGCAGTCTATTACTG | |
| | TGCCGCTGACTTGCACAGA | |
| | CCATACGGTCCAGGAACCC | |
| | AAAGATCCGATGAGTACGA | |
| | TTCTTGGGGTCAGGGAACT | |
| | CAAGTCACTGTCTCTTCAG | |
| | GTGGTGGATCTGGTGGTG | |
| | GAGGTTCAGGTGGTGGAG | |
| | GATCCGGTGGTGGTGGTTC | |
| | TGGTGGTGGTGGATCTGGT | |
| | GGAGGTGAAGTTCAACTTG | |
| | TCGAATCCGGTGGTGCACT | |
| | TGTCCAACCTGGTGGATCT | |
| | CTTAGACTTTCTTGTGCCG | |
| | CCTCCGGTTTTCCTGTTAA | |
| | CCGTTACTCTATGCGTTGG | |
| | TACAGACAAGCCCCTGGAA | |
| | AAGAACGTGAATGGGTTGC | |
| | CGGAATGTCCTCAGCTGGT | |
| | GACAGATCCTCCTACGAAG | |
| | ATTCTGTGAAGGGACGTTT | |
| | CACCATCTCCAGAGATGAC | |
| | GCCCGTAACACCGTTTACC | |
| | TTCAAATGAACTCCCTTAAG | |
| | CCTGAGGATACTGCCGTCT | |
| | ACTATTGTAACGTGAATGT | |
| | CGGATTTGAATACTGGGGA | |
| | CAGGGAACCCAAGTTACTG | |
| | TCTCTTCCGGTGGACATCA | |
| | CCACCACCATCACTAATAG | |
| | (SEQ ID NO: 22) | |
| scR (can be expressed | CAGGAACAACTAATGGAGT | QEQLMESGGGLVTLGGSLKLS |
| under pAOX1, and CYC1 | CTGGGGGTGGTTTGGTTAC | CKASGIDFSHYGISWVRQAPGK |
| can be used as the | CCTGGGTGGTTCTCTTAAG | GLEWIAYIYPNYGSVDYASWVN |
| terminator) (Prielhofer et al. | CTTTCATGTAAGGCCTCTG | GRFTISLDNAQNTVFLQMISLTA |
| 2017) | GTATTGATTTTTCGCACTAC | ADTATYFCARDRGYYSGSRGT |
| | GGTATCTCCTGGGTTAGAC | RLDLWGQGTLVTISSGGGGSG |
| | AAGCTCCTGGAAAAGGTCT | GGGSGGGGSELVMTQTPPSLS |
| | GGAATGGATCGCTTACATT | ASVGETVRIRCLASEFLFNGVS |
| | TACCCAAATTACGGTTCTG | WYQQKPGKPPKFLISGASNLES |
| | TTGACTATGCCTCCTGGGT | GVPPRFSGSGSGTDYTLTIGGV |
| | CAATGGTAGGTTCACTATTT | QAEDVATYYCLGGYSGSSGLT |
| | CCCTTGACAACGCTCAGAA | FGAGTNVEIKGGHHHHHH** |
| | CACGGTATTCCTACAGATG | (SEQ ID NO: 27) |
| | ATCTCCCTAACCGCTGCTG | |
| | ATACTGCAACCTACTTCTGT | |
| | GCTCGTGACAGAGGTTACT | |
| | ACTCTGGCTCTCGTGGAAC | |
| | TAGACTTGACTTATGGGGA | |
| | CAAGGTACTCTCGTTACCA | |
| | TCTCTAGTGGTGGAGGTGG | |
| | TTCTGGAGGAGGAGGTTCC | |
| | GGCGGAGGTGGTAGCGAG | |
| | CTGGTCATGACTCAAACCC | |
| | CTCCATCCCTATCTGCATC | |
| | AGTCGGTGAAACCGTTAGA | |
| | ATTAGATGCCTTGCATCTG | |
| | AGTTCTTGTTCAACGGTGT | |
| | GTCCTGGTATCAACAAAAG | |
| | CCTGGTAAGCCTCCAAAGT | |
| | TTCTCATTTCTGGTGCCTCA | |
| | AACCTCGAATCTGGAGTGC | |
| | CACCAAGATTTTCCGGATC | |
| | TGGCTCTGGTACTGACTAC | |
| | ACTCTGACAATTGGTGGTG | |
| | TTCAAGCTGAGGATGTTGC | |
| | TACCTACTATTGTCTCGGT | |
| | GGTTACTCAGGATCTTCCG | |
| | GCCTAACTTTCGGTGCCGG | |
| | TACAAACGTCGAGATCAAA | |
| | GGTGGACATCACCACCACC | |
| | ATCACTAATAG (SEQ ID NO: | |
| | 23) | |
| SDZ-Fab- HC (can be | GAGGTCCAATTGGTCCAAT | EVQLVQSGGGLVQPGGSLRLS |
| expressed under pAOX1, | CTGGTGGAGGATTGGTTCA | CAASGFTFSHYWMSWVRQAP |
| and CYC1 can be used as | ACCAGGTGGATCTCTGAGA | GKGLEWVANIEQDGSEKYYVD |
| the terminator) (Prielhofer | TTGTCTTGTGCTGCTTCTG | SVKGRFTISRDNAKNSLYLQMN |
| et al. 2017) | GTTTCACCTTCTCTCACTAC | SLRAEDTAVYFCARDLEGLHGD |
| | TGGATGTCATGGGTTAGAC | GYFDLWGRGTLVTVSSASTKG |
| | AAGCTCCTGGTAAGGGTTT | PSVFPLAPCSRSTSESTAALGC |
| | GGAATGGGTTGCTAACATC | LVKDYFPEPVTVSWNSGALTS |
| | GAGCAAGATGGATCAGAGA | GVHTFPAVLQSSGLYSLSSVVT |
| | AGTACTACGTTGACTCTGT | VPSSSLGTKTYTCNVDHKPSNT |
| | TAAGGGAAGATTCACTATTT | KVDKRVESKYGPP** (SEQ ID |
| | CCCGTGATAACGCCAAGAA | NO: 28) |
| | CTCCTTGTACCTGCAAATG | |
| | AACTCCCTTAGAGCTGAGG | |
| | ATACTGCTGTCTACTTCTGT | |
| | GCTAGAGACTTGGAAGGTT | |
| | TGCATGGTGATGGTTACTT | |
| | CGACTTATGGGGTAGAGGT | |
| | ACTCTTGTCACCGTTTCATC | |
| | TGCCTCTACCAAAGGACCT | |
| | TCTGTGTTCCCATTAGCTC | |
| | CATGTTCCAGATCCACCTC | |
| | CGAATCTACTGCAGCTTTG | |
| | GGTTGTTTGGTGAAGGACT | |
| | ACTTTCCTGAACCAGTGAC | |
| | TGTCTCTTGGAACTCTGGT | |
| | GCTTTGACTTCTGGTGTTC | |
| | ACACCTTTCCTGCAGTTTT | |
| | GCAGTCATCTGGTCTGTAC | |
| | TCTCTGTCCTCAGTIGTCA | |
| | CTGTTCCTTCCTCATCTCTT | |
| | GGTACCAAGACCTACACTT | |
| | GCAACGTTGACCATAAGCC | |
| | ATCCAATACCAAGGTTGAC | |
| | AAGAGAGTTGAGTCCAAGT | |
| | ATGGTCCACCTTAATAG | |
| | (SEQ ID NO: 24) | |
| SDZ-Fab- LC (can be | GCTATCCAGTTGACTCAAT | AIQLTQSPSSLSASVGDRVILTC |
| expressed under pDAS1, | CACCATCCTCTTTGTCTGC | RASQGVSSALAWYQQKPGKAP |
| and TDH1 can be used as | TTCTGTTGGTGATAGAGTC | KLLIYDASSLESGVPSRFSGSG |
| the terminator) (Prielhofer | ATCCTGACTTGTCGTGCAT | SGPDFTLTISSLOPEDFATYFC |
| et al. 2017) | CTCAAGGTGTTTCCTCAGC | QQFNSYPLTFGGGTKLEIKRTV |
| | TTTAGCTTGGTACCAACAA | AAPSVFIFPPSDEQLKSGTASV |
| | AAGCCAGGTAAAGCTCCAA | VCLLNNFYPREAKVQWKVDNA |
| | AGTTGCTGATCTACGACGC | LQSGNSQESVTEQDSKDSTYS |
| | TTCATCCCTTGAATCTGGT | LSSTLTLSKADYEKHKVYACEV |
| | GTTCCTTCACGTTTCTCTG | THQGLSSPVTKSFNRGEC** |
| | GATCTGGATCAGGTCCTGA | (SEQ ID NO: 29) |
| | TTTCACTCTGACTATCTCAT | |
| | CCCTTCAACCAGAGGACTT | |
| | TGCTACCTACTTCTGTCAA | |
| | CAGTTCAACTCTTACCCTTT | |
| | GACCTTTGGAGGTGGAACT | |
| | AAGTTGGAGATCAAGAGAA | |
| | CTGTTGCTGCACCATCAGT | |
| | GTTCATCTTTCCTCCATCTG | |
| | ATGAGCAACTGAAGTCTGG | |
| | TACTGCATCTGTTGTCTGC | |
| | TTACTGAACAACTTCTACCC | |
| | AAGAGAAGCTAAGGTCCAA | |
| | TGGAAGGTTGACAATGCCT | |
| | TGCAATCTGGTAACTCTCA | |
| | AGAGTCTGTTACTGAGCAA | |
| | GACTCTAAGGACTCTACTT | |
| | ACTCCCTTTCTTCCACCTTG | |
| | ACTTTGTCTAAGGCTGATTA | |
| | CGAGAAGCACAAGGTTTAC | |
| | GCTTGTGAGGTTACTCACC | |
| | AAGGTTTGTCCTCTCCTGT | |
| | TACCAAGTCTTTCAACAGA | |
| | GGTGAATGCTAATAG (SEQ | |
| | ID NO: 25) | |
| mCherry including FLAG | GTGAGCAAGGGCGAGGAG | VSKGEEDNMAIIKEFMRFKVHM |
| tag, which is highlighted in | GATAACATGGCCATCATCA | EGSVNGHEFEIEGEGEGRPYE |
| grey and bold (can be | AGGAGTTCATGCGCTTCAA | GTQTAKLKVTKGGPLPFAWDIL |
| expressed under pAOX1, | GGTGCACATGGAGGGCTC | VSKGEEDNMAIIKEFMRFKVHM |
| and CYC1 can be used as | CGTGAACGGCCACGAGTTC | EGSVNGHEFEIEGEGEGRPYE |
| the terminator) | GAGATCGAGGGCGAGGGC | GTQTAKLKVTKGGPLPFAWDIL |
| | GAGGGCCGCCCCTACGAG | SPQFMYGSKAYVKHPADIPDYL |
| | GGCACCCAGACCGCCAAG | KLSFPEGFKWERVMNFEDGGV |
| | CTGAAGGTGACCAAGGGT | VTVTQDSSLQDGEFIYKVKLRG |
| | GGCCCCCTGCCCTTCGCCT | TNFPSDGPVMQKKTMGWEAS |
| | GGGACATCCTGTCCCCTCA | SERMYPEDGALKGEIKQRLKLK |
| | GTTCATGTACGGCTCCAAG | DGGHYDAEVKTTYKAKKPVQL |
| | GCCTACGTGAAGCACCCC | PGAYNVNIKLDITSHNEDYTIVE |
| | GCCGACATCCCCGACTACT | 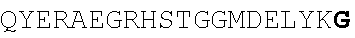 |
| | TGAAGCTGTCCTTCCCCGA GGGCTTCAAGTGGGAGCG CGTGATGAACTTCGAGGAC | 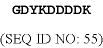 |
| | GGCGGCGTGGTGACCGTG | |
| | ACCCAGGACTCCTCCCTGC | |
| | AGGACGGCGAGTTCATCTA | |
| | CAAGGTGAAGCTGCGCGG | |
| | CACCAACTTCCCCTCCGAC | |
| | GGCCCCGTAATGCAGAAAA | |
| | AGACCATGGGCTGGGAGG | |
| | CCTCCTCCGAGCGGATGTA | |
| | CCCCGAGGACGGCGCCCT | |
| | GAAGGGCGAGATCAAGCA | |
| | GAGGCTGAAGCTGAAGGA | |
| | CGGCGGCCACTACGACGC | |
| | TGAGGTCAAGACCACCTAC | |
| | AAGGCCAAGAAGCCCGTG | |
| | CAGCTGCCCGGCGCCTAC | |
| | AACGTCAACATCAAGTTGG | |
| | ACATCACCTCCCACAACGA | |
| | GGACTACACCATCGTGGAA | |
| | CAGTACGAACGCGCCGAG | |
| | GGCCGCCACTCCACCGGC | |
| | GGCATGGACGAGCTGTACA | |
| | 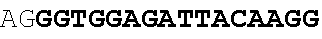 | |
| | 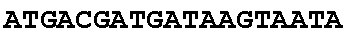 | |
| | 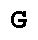 | |
| | (SEQ ID NO: 54) | |

It is also comprised by the present invention that the fusion protein as described herein comprises the elements of the secretion signal, as further described herein, operably linked to the protein of interest as defined herein.

According to a specific aspect, the present invention relates to a nucleic acid molecule encoding a fusion protein comprising:
(a) a secretion signal, the secretion signal comprising
   (I) (i) a signal peptide sequence originating from a KRE1 protein or a signal peptide sequence originating from a SWP1 protein; and
      (ii) an α-mating factor (MFα) pro-sequence;
   or
   (II) a signal peptide sequence originating from a KRE1 protein or a signal peptide sequence originating from a SWP1 protein; and
(b) a protein of interest,
wherein the secretion signal is operably linked to the protein of interest.

### Nucleic Acid Molecules of the Invention

To make use of the secretion signals of the present invention, they may be fused to a protein of interest. Such a fusion protein as described herein may be encoded by a nucleic acid molecule. The nucleic acid molecules of the invention can, e.g., be transformed into a host cell. Accordingly, the present invention relates to a nucleic acid molecule encoding a fusion protein comprising from N-terminus to C-terminus (a) a secretion signal, the secretion signal comprising (i) a signal peptide sequence originating from a KRE1 protein or a signal peptide sequence originating from a SWP1 protein; and (ii) an α-mating factor (MFα) pro-sequence; and (b) a protein of interest.

“Encoding” as used herein means that when the nucleic acid or polynucleotide encoding a protein is expressed, it leads to the production of said protein.

The term “nucleic acid molecule” used herein refers to either DNA or RNA. “Nucleic acid molecule”, “nucleic acid”, “polynucleotide” or simply “nucleotide”, all of which may be used interchangeably, refer to a single or double-stranded polymer of deoxyribonucleotide or ribonucleotide bases read from the 5′ to the 3′ end. It includes both self-replicating plasmids, infectious polymers of DNA or RNA, and non-functional DNA or RNA.

An “expression cassette” as used herein relates to a distinct component of (vector) DNA comprising a gene such as the nucleic acid molecule of the invention and regulatory sequences such as a promoter to be expressed by a transfected cell. The expression cassette may direct the host cell's machinery to express protein(s) of interest. Typically, an expression cassette is composed of one or more genes and the sequences controlling their expression. Accordingly, the present invention further relates to an expression cassette comprising the nucleic acid molecule of the invention and a promoter operably linked thereto. The expression cassette may be in the form of a vector. The expression cassette may be comprised by a vector.

In other embodiments, the nucleic acid molecule of the invention and/or a polynucleotide encoding one or more component(s) of a SRP can be integrated in a plasmid or vector. Accordingly, the present invention also relates to a vector comprising the nucleic acid of the invention. The term “plasmid” may relate to a DNA molecule used as a vehicle to artificially carry foreign genetic material into another cell, where it can be replicated and/or expressed (e.g., plasmid, cosmid, Lambda phages). A vector containing foreign DNA is termed recombinant DNA. Vectors include, but are not limited to, plasmids, viral vectors, cosmids, and artificial chromosomes, preferably plasmids. The vector itself may generally be a DNA sequence that consists of an insert (transgene) and a larger sequence that serves as the “backbone” of the vector. All vectors may be used for cloning and can therefore be seen as cloning vectors, but there are also vectors designed specially for cloning, while others may be designed specifically for other purposes, such as transcription and protein expression. Vectors designed specifically for the expression of the transgene in the target cell are called expression vectors, and generally have a promoter sequence that drives expression of the transgene. A skilled person is able to employ suitable plasmids or vectors depending on the host cell used.

Preferably, the vector is a eukaryotic expression vector, preferably a yeast expression vector.

Preferably, the vector is a eukaryotic expression vector, preferably a yeast expression vector. Examples of vector using yeast as a host include Ylp type vector, YEp type vector, YRp type vector, YCp type vector, pGPD-2, pAO815, pGAPZ, pGAPZα, pHIL-D2, pHIL-S1, pPIC3.5K, pPIC9K, pPICZ, pPICZα, pPIC3K, pHWO10, pPUZZLE and 2 μm plasmids. Such vectors are known and are for example described in Cregg et al., Mol Biotechnol. (2000) 16(1): 23-52. Preferably, the vector is a pPM2dZ30 vector (described in W02008/128701A2). Alternatively, the vector is a Golden Gate-based GoldenPiCS, consisting of the backbones BB1, BB2 and BB3aK/BB3eH/BB3rN (Prielhofer et al., 2017).

Vectors can be used for the transcription of cloned recombinant nucleotide sequences, i.e. of recombinant genes and the translation of their mRNA in a suitable host organism. Vectors can also be used to integrate a target polynucleotide into the host cell genome by methods known in the art, such as described by J. Sambrook et al., Molecular Cloning: A Laboratory Manual (3rd edition), Cold Spring Harbor Laboratory, Cold Spring Harbor Laboratory Press, New York (2001) or Stearns et al. (1990), Methods in Enzymology, 185: 280-297. A “vector” usually comprises an origin for autonomous replication in the host cells, preferably for both a bacterial origin and a eukaryotic origin for the host cells of the invention, selectable markers, a number of restriction enzyme cleavage sites, a suitable promoter sequence and a transcription terminator, which components are operably linked together. The polypeptide coding sequence of interest is operably linked to transcriptional and translational regulatory sequences that provide for expression of the polypeptide in the host cells.

Large numbers of suitable plasmids or vectors are known to those of skill in the art and many are commercially available. Examples of suitable vectors are provided in Sambrook et al, eds., Molecular Cloning: A Laboratory Manual (2nd Ed.), Vols. 1-3, Cold Spring Harbor Laboratory (1989), and Ausubel et al, eds., Current Protocols in Molecular Biology, John Wiley & Sons, Inc., New York (1997).

A vector or plasmid of the present invention encompass yeast artificial chromosome, which refers to a DNA construct that can be genetically modified to contain a heterologous DNA sequence (e.g., a DNA sequence as large as 3000 kb), that contains telomeric, centromeric, and origin of replication (replication origin) sequences.

The sequence encoding the fusion protein or the secretion signal of the invention may be operably linked to a promoter. The term “promoter” as used herein refers to a region that facilitates the transcription of a particular gene. A promoter typically increases the amount of recombinant product expressed from a nucleotide sequence as compared to the amount of the expressed recombinant product when no promoter exists. A promoter from one organism can be utilized to enhance recombinant product expression from a sequence that originates from another organism. The promoter can be integrated into a host cell chromosome by homologous recombination using methods known in the art (e.g. Datsenko et al, Proc. Natl. Acad. Sci. U.S.A., 97(12): 6640-6645 (2000)). In addition, one promoter element can increase the amount of products expressed for multiple sequences attached in tandem. Hence, one promoter element can enhance the expression of one or more recombinant products.

The promoter could be an “inducible promoter” or “constitutive promoter.” “Inducible promoter” refers to a promoter which can be induced by the presence or absence of certain factors, and “constitutive promoter” refers to an unregulated promoter that allows for continuous transcription of its associated gene or genes.

In a preferred embodiment, the nucleotide sequences encoding the fusion protein of the invention or the secretion signal of the invention are driven by an inducible promoter.

Many inducible promoters are known in the art. Many are described in a review by Gatz, Curr. Op. Biotech., 7: 168 (1996) (see also Gatz, Ann. Rev. Plant. Physiol. Plant Mol. Biol., 48: 89 (1997)). Examples include tetracycline repressor system, Lac repressor system, copper-inducible systems, salicylate-inducible systems (such as the PR1 a system), glucocorticoid-inducible (Aoyama et al., 1997), alcohol-inducible systems, e.g., AOX promoters, and ecdysome-inducible systems. Also included are the benzene sulphonamide-inducible (U.S. Pat. No. 5,364,780) and alcohol-inducible (WO 97/06269 and WO 97/06268) inducible systems and glutathione S-transferase promoters.

Suitable promoter sequences for use with yeast host cells are described in Mattanovich et al., Methods Mol. Biol. (2012) 824:329-58 and include glycolytic enzymes like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPG!), the 3-phosphoglycerate kinase promoter (PPGK), the glycerol aldehyde phosphate dehydrogenase promoter (PGAP), translation elongation factor promoter (PTEF), and the promoters of P. pastoris enolase 1 (PENO1), triose phosphate isomerase (PTPI), ribosomal subunit proteins (PRPS2, PRPS7, PRPS31, PRPL1), alcohol oxidase promoter (PAOX) or variants thereof with modified characteristics, the formaldehyde dehydrogenase promoter (PFLD), isocitrate lyase promoter (PICL), alpha-ketoisocaproate decarboxylase promoter (PTHI), the promoters of heat shock protein family members (PSSA1, PHSP90, PKAR2), 6-Phosphogluconate dehydrogenase (PGND1), phosphoglycerate mutase (PGPM1), transketolase (PTKL1), phosphatidylinositol synthase (PPIS1), ferro-O2-oxidoreductase (PFET3), high affinity iron permease (PFTR1), repressible alkaline phosphatase (PPHO8), N-myristoyl transferase (PNMT1), pheromone response transcription factor (PMCM1), ubiquitin (PUB14), single-stranded DNA endonuclease (PRAD2), the promoter of the major ADP/ATP carrier of the mitochondrial inner membrane (PPET9) (WO2008/128701) and the formate dehydrogenase (FMD) promoter. The GAP promoter, AOX promoter or a promoter originating from GAP or AOX promoter is particularly preferred. AOX promoters can be induced by methanol and are repressed by glucose. Promoters originating from the AOX promoter for methanol-induced as well as methanol-free production are described in WO 2006/089329, EP 1851312 B1 and EP 2199389 B1. Carbon source regulable promoter, e.g., de-repressible promoters such as described in WO2013050551 (e.g., pG1-pG8, fragments of pG1, designated pG1a-pG1f) and WO2017021541 (e.g., pG1-D1240, or pG1-D1427) may be used. Further examples are constitutive promoter, such as MDH3, PORI, PDC1, FBA1-1, or GPM1 (Prielhofer et al. 2017, BMC Sys Biol. 11: 123), or as disclosed in WO2014139608 (e.g., pCS1).

Further examples of suitable promoters include Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase (PGK), and the maltase gene promoter (MAL).

Other useful promoters for yeast host cells are described by Romanos et al, 1992, Yeast 8: 423-488.

### Host Cells of the Invention

As used herein, a “host cell” refers to a cell which is capable of protein expression and protein secretion. Such a host cell can be applied in the methods of the present invention. For that purpose, for the host cell to express a polypeptide, a nucleic acid molecule encoding the fusion protein is present or introduced in the cell. Host cells provided by the present invention can be eukaryotes. As will be appreciated by one of skill in the art, a prokaryotic cell lacks a membrane-bound nucleus, while a eukaryotic cell has a membrane-bound nucleus. Examples of eukaryotic cells include, but are not limited to, vertebrate cells, mammalian cells, human cells, animal cells, invertebrate cells, plant cells, nematodal cells, insect cells, stem cells, fungal cells or yeast cells. Preferably, the host cell is a yeast cell.

Accordingly, the present invention relates to a host cell comprising the nucleic acid molecule of the invention. Additionally, the present invention relates to a host cell comprising the expression cassette of the invention. The present invention further relates to a host cell comprising the vector of the invention.

Examples of yeast cells include but are not limited to the Saccharomyces genus (e.g. Saccharomyces cerevisiae, Saccharomyces kluyveri, Saccharomyces uvarum, Saccharomyces paradoxus, Saccharomyces eubayanus, Saccharomyces kudriavzevii), the Komagataella genus (Komagataella pastoris, Komagataella pseudopastoris or Komagataella phaffii), Kluyveromyces genus (e.g. Kluyveromyces lactis, Kluyveromyces marxianus), the Candida genus (e.g. Candida utilis, Candida cacaoi), the Geotrichum genus (e.g. Geotrichum fermentans), as well as Hansenula polymorpha and Yarrowia lipolytica. Accordingly, the eukaryotic host cell of the invention or the eukaryotic host cell used in the methods and uses of the invention can be a fungal or yeast host cell, preferably a yeast host cell, selected from the group consisting of Komagataella phaffii (Pichia pastoris), Hansenula polymorpha, Saccharomyces cerevisiae, Kluyveromyces lactis, Yarrowia lipolytica, Pichia methanolica, Candida boidinii, Komagataella spp. and Schizosaccharomyces pombe, or a fungal host cell such as Trichoderma reesei, Aspergillus niger.

The genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris and Komagataella phaffii. Therefore Pichia pastoris is synonymous for both Komagataella pastoris and Komagataella phaffii, preferably synonymous for Komagataella phaffii.

Examples for Pichia pastoris strains useful in the present invention are X33 and its subtypes GS115, KM71, KM71H; CBS7435 (mut+) and its subtypes CBS7435 mut^{s}, CBS7435 mut^{s}ΔArg, CBS7435 mut^{s}ΔHis, CBS7435 mut^{s}ΔArg, ΔHis, CBS7435 mut^{s} PDF⁺, CBS 704 (=NRRL Y-1603=DSMZ 70382), CBS 2612 (=NRRL Y-7556), CBS 9173-9189 and DSMZ 70877 as well as mutants thereof. Preferably, the host cell is P. pastoris CBS7435 mut^{s} or a subtype thereof, more preferably P. pastoris CBS7435 mut^{s}.

According to a further preferred embodiment, the host cell is a Pichia pastoris, Hansenula polymorpha, Trichoderma reesei, Saccharomyces cerevisiae, Kluyveromyces lactis, Yarrowia lipolytica, Pichia methanolica, Candida boidinii, and Komagataella, and Schizosaccharomyces pombe. It may also be a host cell from Ustilago maydis.

As used herein, “recombinant” refers to the alteration of genetic material by human intervention. Typically, recombinant refers to the manipulation of DNA or RNA in a virus, cell, plasmid or vector by molecular biology (recombinant DNA technology) methods, including cloning and recombination. A recombinant cell, polypeptide, or nucleic acid can be typically described with reference to how it differs from a naturally occurring counterpart (the “wild-type”). A “recombinant cell” or “recombinant host cell” refers to a cell or host cell that has been genetically altered to comprise a nucleic acid sequence which was not native to said cell.

The term “manufacture” or “manufacturing” as used presently refers to the process in which the protein of interest is expressed. A “host cell for manufacturing a protein of interest” refers to a host cell in which nucleic acid sequences encoding a protein of interest may be introduced. The recombinant host cell within the present invention does not necessarily contain the nucleic acid sequences encoding a protein of interest. It is appreciated by a skilled person in the art that the host cells can be provided for inserting desired nucleotide sequences into the host cell, for example, in a kit.

The terms “polypeptide” and “protein” are interchangeably used. The term “polypeptide” refers to a protein or peptide that contains two or more amino acids, typically at least 3, preferably at least 20, more preferred at least 30, such as at least 50 amino acids. Accordingly, a polypeptide comprises an amino acid sequence, and, thus, sometimes a polypeptide comprising an amino acid sequence is referred to herein as a “polypeptide comprising a polypeptide sequence”. Thus, herein the term “polypeptide sequence” is interchangeably used with the term “amino acid sequence”.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Systems to express proteins of interest comprising synthetic amino acids or amino acid analogs are known to a person skilled in the art and include, but are not limited to, the use of an expanded genetic code. The key prerequisites to expand the genetic code are: a non-standard amino acid to encode, an unused codon to adopt, a tRNA that recognises this codon, and a tRNA synthetase that recognises only that tRNA and only the non-standard amino acid. Amino acid mimetics refers to chemical compounds that have a structure that is different from the general chemical structure of an amino acid, but that function in a manner similar to a naturally occurring amino acid.

To further increase the secretion of the fusion protein or the protein of interest after the cleavage of the secretion signal described herein (see Examples 4-9), the (recombinant) host cell may additionally be engineered to overexpress one or more component(s) of the signal recognition particle (SRP).

The signal recognition particle (SRP) as used herein relates to an abundant, cytosolic, universally conserved ribonucleoprotein (protein-RNA complex) that recognizes and targets specific proteins to the endoplasmic reticulum in eukaryotes and the plasma membrane in prokaryotes. In eukaryotes, SRP binds to the signal peptide sequence—such as the signal peptide sequence originating from a SWP1 or KRE1 protein—of a newly synthesized peptide as it emerges from the ribosome. This binding leads to the slowing of protein synthesis known as “elongation arrest”, a conserved function of SRP that facilitates the coupling of the protein translation and the protein translocation processes. SRP then targets this entire complex (the ribosome-nascent chain complex) to the protein-conducting channel, also known as the translocon, in the ER (Endoplasmic reticulum) membrane. This occurs via the interaction and docking of SRP with its cognate SRP receptor that is located in close proximity to the translocon.

In eukaryotes there are three domains between SRP and its receptor that function in guanosine triphosphate (GTP) binding and hydrolysis. These are located in two related subunits in the SRP receptor (SRα and SRβ) and the SRP protein SRP54. Upon docking, the nascent peptide chain is inserted into the translocon channel where it enters into the ER. Protein synthesis resumes as SRP is released from the ribosome. The SRP-SRP receptor complex dissociates via GTP hydrolysis and the cycle of SRP-mediated protein translocation continues. Thus, the SRP-dependent translocation can in some cases also be seen as a co-translational mode of translocation. In one specific embodiment, the translocation of the fusion protein of the invention into the ER is co-translational.

Once inside the ER, the signal peptide sequence can be cleaved from the core protein by signal peptidase. Signal peptide sequences are therefore not a part of mature proteins such as the secreted protein of interest after cleavage of the secretion signal from the fusion protein of the invention.

The SRP may comprise SRP68, SRP72, SRP9-21, SRP54, SRP14, Sec65 and 7SL RNA. Accordingly, the “one or more component(s) of the SRP” may relate to at least one selected from the group consisting of SRP68, SRP72, SRP9-21, SRP54, SRP14, Sec65 and 7SL RNA. In a preferred embodiment, all components of the SRP, i.e. SRP68, SRP72, SRP9-21, SRP54, SRP14, Sec65 and 7SL RNA are overexpressed in the host cell. Advantageously, the one ore more component(s) of the SRP overexpressed in the host cell originate from the same species as the host cell. However, also encompassed by the invention is that heterologous one or more component(s) of the SRP may be overexpressed. Further encompassed are that functional homologs of the one more component(s) of the SRP are overexpressed, including, but not limited to, functional homologs of one or more of SRP68, SRP72, SRP9-21, SRP54, SRP14, Sec65 and 7SL RNA, preferably of Komagataella phaffii; or functional homologs of SRP68, SRP72, SRP9-21, SRP54, SRP14, Sec65 and 7SL RNA, preferably of Komagataella phaffii.

Exemplary sequences of each of the SRP components originating from Komagataella phaffii are listed in the following Table 3.

TABLE 3
Exemplary sequences of each of the SRP components that may be overexpressed in
the host cell.

| SRP Subunit | Sequence (5′-3′) | Protein sequence |
|---|---|---|
| SRP68 (Expressed under | ATGGAATCGCCCTTGCAATCTA | MESPLQSTYGERAERYLDS |
| pADH2, and RPL2att was | CATACGGAGAAAGAGCCGAAA | ADAFHKQRHRLNRRLHKLR |
| used as the terminator) | GGTATTTAGATAGTGCTGATGC | KSLDIHVTDTKNYREKEQISK |
| (Prielhofer et al. 2017) | TTTTCATAAACAAAGACACAGAT | IDLESYNRDKRYGDIILFTAE |
| | TGAATCGAAGGCTGCACAAGTT | RDHMYSDEVKEIMKVHHSK |
| | ACGTAAGAGCTTGGATATTCAT | SREKFIVSRLKRSLDHGRKL |
| | GTTACTGATACTAAGAACTATA | LILVGDEPDEMRKLEVFVYV |
| | GAGAGAAAGAGCAGATTTCCAA | ALIQGKLSIANKNWTNAQYA |
| | AATTGATCTAGAGTCGTACAAC | LSVARCGLQFLDKYGTETQT |
| | AGGGATAAGCGATATGGTGACA | DLYNGIIDTHIDQMLKFVIYQ |
| | TTATACTGTTCACTGCAGAGAG | ATKNNSPILDTECRHQIRTDT |
| | GGATCACATGTATAGTGATGAG | LGYLDQARQIIESKDPEFLNV |
| | GTCAAGGAGATCATGAAGGTCC | GVVETQLIWWDYDISIHSEE |
| | ATCATAGTAAATCGAGAGAAAA | VAKLISDANEKLQLIEDGNVS |
| | GTTTATTGTTTCTAGATTGAAGA | SYDPALLTLQEALDAHQLLM |
| | GATCACTGGACCACGGTAGAAA | ARNVDNFADDDQNNHVLLS |
| | ATTACTGATCCTAGTTGGAGAC | YIRYLLLITTLRRDITLIDQVR |
| | GAGCCTGATGAGATGAGAAAAT | NRSVVNSSLAVALERAKDVG |
| | TGGAAGTATTTGTTTATGTTGCA | RIFDNIVKKVNELKDVPGVYN |
| | TTGATTCAGGGTAAACTTTCCAT | KQEEWNSLQALDAYFQASKI |
| | TGCAAACAAGAATTGGACCAAT | QHLASTHLLFNRSKESLALLI |
| | GCTCAGTATGCTCTCAGTGTGG | KAKSLVKGHTIAGEYPTNFP |
| | CGAGATGTGGGCTCCAGTTTTT | TNKDLSSILEQINQDILKAYVL |
| | GGACAAATATGGTACTGAAACA | AKYKQESSLGGVSEYDFIAD |
| | CAAACTGACCTCTATAATGGCA | NRNKVPSNPSLHKIASVSYK |
| | TAATTGACACTCACATAGATCAA | NVKPVNVKPVLFDIAFNYVS |
| | ATGTTGAAATTTGTGATCTACCA | QPNQIFEEPIESSNKQERQA |
| | AGCTACTAAAAATAACAGTCCT | DSESPSPEKKKKGLFGLFR** |
| | ATTTTGGATACAGAGTGCAGAC | (SEQ ID NO: 5) |
| | ATCAAATTAGGACGGACACCCT | |
| | AGGGTATTTGGATCAGGCAAGG | |
| | CAAATAATAGAATCAAAAGATC | |
| | CCGAGTTTCTGAATGTTGGAGT | |
| | TGTTGAAACTCAGTTGATTTGG | |
| | TGGGACTACGATATCTCTATTC | |
| | ATTCAGAGGAGGTAGCAAAGCT | |
| | GATTTCAGATGCGAACGAAAAG | |
| | CTGCAACTTATCGAGGATGGAA | |
| | ACGTCTCCTCATATGATCCGGC | |
| | TCTACTAACTCTTCAAGAAGCG | |
| | CTGGATGCTCATCAGTTGTTGA | |
| | TGGCCAGAAATGTTGACAACTT | |
| | CGCAGACGACGATCAAAACAAT | |
| | CATGTTTTACTGTCGTACATCA | |
| | GATATTTGTTACTTATCACCACT | |
| | TTGAGAAGGGACATTACTTTGA | |
| | TAGACCAAGTTAGAAACAGATC | |
| | TGTGGTTAATTCTTCCCTAGCT | |
| | GTGGCTCTGGAACGTGCTAAAG | |
| | ACGTTGGTAGAATTTTCGACAA | |
| | TATCGTCAAGAAAGTCAATGAG | |
| | TTGAAAGACGTTCCAGGTGTTT | |
| | ACAACAAGCAAGAGGAGTGGA | |
| | ATTCGTTGCAGGCATTGGATGC | |
| | TTATTTCCAAGCATCCAAGATC | |
| | CAACATTTGGCATCTACCCACC | |
| | TTTTATTCAACAGATCCAAGGAA | |
| | TCATTGGCGTTATTAATAAAGG | |
| | CAAAGTCCTTGGTAAAGGGGCA | |
| | CACTATCGCCGGAGAATATCCC | |
| | ACTAATTTCCCTACGAATAAAGA | |
| | TTTGAGTTCGATCTTAGAACAAA | |
| | TTAATCAAGACATCCTTAAGGC | |
| | TTATGTTTTGGCCAAGTATAAG | |
| | CAAGAGTCCTCTTTAGGTGGTG | |
| | TATCGGAGTATGATTTCATTGCT | |
| | GACAATCGCAACAAGGTTCCGT | |
| | CGAATCCCAGTCTGCACAAGAT | |
| | TGCCTCTGTATCCTACAAGAAT | |
| | GTCAAACCTGTCAATGTTAAGC | |
| | CTGTATTGTTTGACATAGCTTTC | |
| | AACTACGTGAGTCAACCAAACC | |
| | AGATATTTGAGGAACCTATCGA | |
| | GAGTTCAAACAAGCAAGAGAGA | |
| | CAAGCGGATTCTGAATCTCCGT | |
| | CACCAGAGAAGAAAAAAAAGGG | |
| | ATTGTTTGGATTGTTCCGCTAG | |
| | TAG (SEQ ID NO: 12) | |
| SRP72 (Expressed under | ATGTCGTCTCTTTCAGAGCTGG | MSSLSELVSELAIHSEKRQY |
| pPOR1, and RPP1btt | TATCGGAATTGGCAATCCATTC | KEAYEKAKRIIDLGHPLDLDT |
| was used as the | TGAGAAGAGGCAGTACAAAGAA | LKLGLVASINLDQYHNAGRLI |
| terminator) (Prielhofer | GCATATGAGAAAGCAAAGCGCA | SKSKDHIVYDGMKEFLLLIGY |
| et al. 2017) | TTATAGATTTGGGCCACCCTCT | VYYKNGDSKNFETLLKDSAF |
| | TGACCTTGACACATTGAAGCTA | QGRAFEHLKAQYYYKIGENE |
| | GGTTTGGTGGCTTCGATCAACC | KALKIYRELSKNPLDEVVDLS |
| | TGGACCAATATCACAACGCAGG | VNERAVISQLLELDGVVEQP |
| | TCGCCTCATATCAAAAAGTAAG | VSRPIDDSYDCKFNDALYQV |
| | GATCATATCGTATATGATGGAA | KIGDYESALDLLEEAKAICEE |
| | TGAAGGAATTCTTGCTATTAATT | NTKDLPLDTREAEIVPILLQIA |
| | GGATATGTGTACTACAAGAACG | YVKQLKGKKEESLTALRSLS |
| | GAGACTCGAAGAATTTTGAAAC | KPKDSLLDLIYRNNLLSLRID |
| | TCTACTAAAGGATTCAGCTTTC | EYGRNDTNFHILYRELGFPN |
| | CAAGGAAGAGCATTTGAACACC | SIDINKDKLTVSQRVALTRNE |
| | TCAAAGCCCAATACTATTACAA | SLLALELGKIPSQSDLKLFYD |
| | GATCGGGGAGAACGAAAAGGC | ATSEFLDLNTKLEASMIYNYF |
| | ACTCAAGATTTACCGAGAGCTA | MRRPGQQEVPNALLTAQLAI |
| | TCTAAGAACCCATTGGATGAAG | NVGNINNARTVLETVVSNDE |
| | TTGTAGACTTGAGTGTCAATGA | KNLLEPSIVVSLYLIYDKLQS |
| | AAGAGCTGTCATTAGCCAGCTT | GRLQVELLKKVADLLLESEIS |
| | TTGGAATTGGATGGTGTCGTTG | STQQRKFFKDIAFKTLNHDA |
| | AACAGCCTGTATCTCGACCAAT | VLANRLFEKLHSIYPNDELVS |
| | AGACGACTCATACGATTGCAAA | TYLNSSSNASNNNTTTTNFS |
| | TTCAACGATGCCCTTTACCAGG | ELDDLVLGIDTDKLISEGFDT |
| | TAAAGATTGGTGACTATGAATC | FESSKRPTTIISSTNKKRRTR |
| | TGCATTGGATCTCTTGGAAGAA | LKPKHEAKEKYKRLDEERWL |
| | GCCAAAGCCATATGTGAAGAAA | PLKDRSYYRPKKGKKIRNTT |
| | ATACAAAAGATCTGCCTTTGGA | QGTVTSNTSEISGLKKTLPK |
| | CACACGAGAAGCAGAGATTGTT | KSSKKKGRK** |
| | CCTATTCTGTTACAAATTGCTTA | (SEQ ID NO: 6) |
| | CGTCAAACAACTTAAGGGTAAA | |
| | AAGGAAGAATCCTTGACTGCGC | |
| | TGAGAAGTCTTTCTAAACCAAA | |
| | GGACTCTCTTTTAGATCTTATTT | |
| | ACAGAAATAACTTACTGTCATTA | |
| | AGGATTGATGAATACGGAAGAA | |
| | ATGATACCAACTTTCATATTCTT | |
| | TATCGTGAGTTAGGATTCCCTA | |
| | ATTCGATAGACATTAATAAAGAC | |
| | AAGTTGACAGTGTCCCAAAGGG | |
| | TTGCGTTGACCAGAAATGAATC | |
| | ATTATTGGCACTCGAGCTTGGA | |
| | AAGATCCCATCTCAAAGTGATC | |
| | TCAAGCTCTTTTATGATGCTACT | |
| | TCGGAATTTTTAGATTTGAACAC | |
| | CAAGCTAGAAGCCTCAATGATT | |
| | TATAATTATTTCATGAGACGCCC | |
| | TGGCCAGCAAGAGGTTCCAAAT | |
| | GCTCTTTTAACTGCACAGCTGG | |
| | CTATCAACGTTGGTAACATCAA | |
| | TAATGCAAGAACTGTTCTTGAA | |
| | ACTGTGGTGTCGAACGACGAAA | |
| | AGAATTTACTAGAACCATCTATT | |
| | GTGGTATCTTTGTATTTGATTTA | |
| | CGATAAGCTTCAAAGCGGAAGA | |
| | TTGCAAGTTGAACTCCTGAAGA | |
| | AAGTAGCGGATCTTTTGCTAGA | |
| | ATCAGAGATTTCCAGCACTCAA | |
| | CAACGTAAGTTTTTCAAAGATAT | |
| | TGCCTTCAAAACCCTCAATCAC | |
| | GATGCAGTTTTGGCCAATCGAC | |
| | TATTTGAGAAACTGCATAGTATT | |
| | TACCCTAATGATGAGTTGGTAT | |
| | CCACGTACTTGAACTCTTCATC | |
| | TAATGCATCCAACAATAACACTA | |
| | CCACGACCAACTTCTCCGAATT | |
| | GGACGACTTGGTCCTAGGCATA | |
| | GACACGGATAAGCTTATTAGTG | |
| | AAGGATTTGATACTTTTGAGTC | |
| | CAGCAAAAGACCGACCACGATT | |
| | ATCAGCTCAACTAACAAGAAAC | |
| | GTCGTACTAGATTGAAGCCCAA | |
| | ACATGAAGCCAAGGAAAAGTAT | |
| | AAGCGTCTGGATGAGGAAAGAT | |
| | GGTTACCTTTAAAGGACCGCAG | |
| | TTATTACAGACCCAAAAAGGGA | |
| | AAGAAAATTAGAAATACCACTC | |
| | AGGGTACTGTCACTAGTAATAC | |
| | TAGTGAAATAAGTGGCTTGAAA | |
| | AAGACTCTGCCAAAGAAAAGTT | |
| | CCAAAAAGAAAGGAAGAAAATG | |
| | ATAG (SEQ ID NO: 13) | |
| SRP9-21 (Expressed | ATGCCTCCTGTGAAATCTCTGG | MPPVKSLDIFFNRTEKLLEAN |
| under pPDC1, and | ACATCTTTTTCAACCGCACAGA | PTTTKVSIKLGVNFNDHENP |
| RPS25att was used as | GAAGCTCTTAGAAGCCAACCCC | QSKHNVITVRVSDPVSGSNF |
| the terminator) | ACAACGACAAAAGTTTCCATCA | KFKVTNKTDMLKIFSFLGPH |
| (Prielhofer et al. 2017) | AATTGGGCGTAAATTTCAATGA | GIELPISGQQSQIKSNDQTQ |
| | TCACGAGAATCCTCAAAGCAAG | SDNTEVPTTFHRGATSILAN |
| | CACAACGTCATAACGGTGAGAG | KAFEKKPLIIKDSSTAKKGGK |
| | TATCTGATCCAGTGAGCGGGTC | GGKKKGKKF* (SEQ ID |
| | CAATTTCAAATTCAAAGTGACCA | NO: 7) |
| | ATAAAACTGATATGCTGAAAATA | |
| | TTCAGTTTCTTAGGTCCTCATG | |
| | GCATTGAGTTACCAATTTCTGG | |
| | CCAGCAAAGCCAGATAAAGAGT | |
| | AATGATCAGACTCAGAGTGACA | |
| | ATACTGAAGTGCCTACCACATT | |
| | TCATAGGGGAGCCACCAGTATT | |
| | TTGGCTAATAAGGCATTTGAGA | |
| | AGAAACCACTGATTATTAAGGA | |
| | TTCAAGTACCGCAAAGAAAGGT | |
| | GGTAAAGGTGGTAAGAAGAAG | |
| | GGTAAGAAATTTTAA (SEQ ID | |
| | NO: 14) | |
| SEC65 (Expressed under | ATGCCATTACTAGAGGAAATAA | MPLLEEISDAEDIDNLEMDLA |
| pRPP1b, and RPS17btt | GTGATGCAGAGGACATAGACAA | EFDPTLRTPIAEQRPAPQVV |
| was used as the | CTTGGAGATGGATTTAGCCGAG | RSQDAESGQTPLVPNQDQI |
| terminator) (Prielhofer | TTTGATCCTACTTTAAGGACTCC | SQYIEQFKEGGTINKDQVIRP |
| et al. 2017) | GATAGCTGAGCAAAGACCAGCT | DEMMEKEMAELKSFQILYPC |
| | CCTCAGGTTGTCAGATCACAAG | YFDKNRSVKEGRRCQKEYG |
| | ATGCCGAAAGTGGACAGACTCC | VENPLAKTILDACRYLDIPCIL |
| | TTTGGTTCCTAACCAGGATCAA | EPEKTHPQDFGNPGRVRVAI |
| | ATAAGTCAGTATATTGAACAATT | KESGKYLDEQYKTKRKLIQL |
| | CAAAGAAGGTGGCACCATAAAC | VGQFLVEHPTTLQKVQELPG |
| | AAGGATCAAGTGATTAGACCCG | PPELQQGGYIPERVPRVKGL |
| | ACGAAATGATGGAAAAAGAAAT | KMNEIVPLHSPFTIKHPSTKS |
| | GGCAGAGTTGAAAAGCTTCCAA | VYEREPEPAPPAAVPKAPKQ |
| | ATTTTGTACCCATGTTACTTTGA | KKIMVRR** (SEQ ID NO: 8) |
| | TAAAAATAGAAGTGTTAAAGAA | |
| | GGAAGAAGATGCCAAAAGGAG | |
| | TATGGTGTGGAGAACCCCCTG | |
| | GCAAAGACAATATTAGATGCTT | |
| | GCAGGTACTTGGATATACCTTG | |
| | CATCCTGGAGCCTGAAAAGACT | |
| | CATCCTCAAGATTTTGGTAATC | |
| | CAGGAAGAGTGAGAGTGGCTA | |
| | TCAAGGAGAGTGGGAAGTATCT | |
| | CGATGAACAATATAAGACCAAA | |
| | AGGAAACTAATACAGTTGGTAG | |
| | GACAATTTCTGGTTGAACATCC | |
| | AACAACGTTACAGAAAGTTCAA | |
| | GAATTGCCCGGTCCACCTGAGT | |
| | TGCAACAGGGCGGGTACATTC | |
| | CAGAACGTGTACCCCGAGTGAA | |
| | AGGGTTAAAGATGAACGAAATT | |
| | GTTCCTTTGCATTCGCCATTCA | |
| | CTATTAAGCATCCAAGTACTAAA | |
| | TCTGTTTATGAAAGGGAACCTG | |
| | AGCCCGCACCACCCGCCGCCG | |
| | TGCCCAAAGCTCCGAAACAGAA | |
| | GAAAATAATGGTGAGAAGATAA | |
| | TAG (SEQ ID NO: 15) | |
| SRP54 (Expressed under | ATGGTATTGGCAGATCTTGGAA | MVLADLGRRINNAVGNVTKS |
| pFBA1-1, and RPS2tt | GGCGTATCAATAACGCCGTTGG | NVVDADVISNMLKEICNALLE |
| was used as the | AAATGTCACCAAGTCCAATGTT | SDVNIKLVAQLREKIRKQIDA |
| terminator) (Prielhofer | GTTGACGCTGACGTCATCAGCA | EDKPGINKKKLIQKVVFDELV |
| et al. 2017) | ACATGTTAAAGGAGATTTGTAA | KLVDCNEAELFKPKKKQTNV |
| | CGCCCTATTGGAGTCCGATGTG | IMMVGLQGAGKTTTCTKLAV |
| | AACATTAAACTAGTTGCCCAATT | YYQRRGFKVGMVCGDTFRA |
| | GAGAGAGAAAATACGAAAACAG | GAFDQLKQNATKAKIPYYGS |
| | ATCGACGCAGAGGATAAACCAG | YTETDPVKVTFDGVEEFRKE |
| | GAATTAATAAGAAGAAGCTGAT | KFEIIIVDTSGRHRQEEDLFE |
| | CCAGAAGGTCGTTTTTGATGAG | EMVQIGKAIKPNQTIMVLDAS |
| | CTGGTGAAACTTGTTGATTGCA | IGQSAESQSKAFKESSDFGA |
| | ACGAAGCTGAGCTGTTCAAGCC | IIITKMDSNSKGGGALSAIAAT |
| | AAAGAAAAAACAGACGAATGTG | NTPVAFIATGEHIQNFEKFSG |
| | ATCATGATGGTCGGTTTACAAG | RGFISKLLGIGDIEGLMEHVQ |
| | GTGCTGGTAAGACAACAACCTG | SMNLDQGDTIKNFKEGKFTL |
| | TACTAAACTGGCAGTGTATTAC | QDFQTQLNNIMKMGPLSKLA |
| | CAGAGAAGAGGATTCAAAGTGG | QMLPGGMGQLMGQVGEEE |
| | GAATGGTCTGTGGTGACACTTT | ASKRLKRMIYIMDSMTKQEL |
| | CCGAGCTGGTGCGTTTGACCA | SSDGRLFIDQPSRMVRVAR |
| | GCTGAAACAAAACGCTACCAAG | GSGTSVTEVELVLLQQKMM |
| | GCTAAGATTCCCTACTATGGTT | ARMALQSKNMMSGAGGPA |
| | CATATACAGAAACTGACCCTGT | GMASKMNPANMRRAMQQM |
| | GAAAGTGACCTTTGATGGTGTG | QSNPGMMDNMMNMFGGA |
| | GAAGAATTCAGGAAGGAAAAGT | GGAGGAGMPDMQEMMKQ |
| | TTGAAATAATAATTGTGGATACT | MSSGQMKMPSQQEMMSM |
| | TCTGGTAGACACAGGCAGGAG | MKQFGMG** (SEQ ID NO: 9) |
| | GAAGATTTATTCGAAGAGATGG | |
| | TACAAATTGGAAAAGCTATCAA | |
| | GCCTAATCAAACAATCATGGTA | |
| | CTGGATGCTTCCATAGGTCAAT | |
| | CTGCCGAATCTCAATCTAAAGC | |
| | ATTTAAGGAATCATCCGATTTTG | |
| | GTGCCATTATCATAACTAAAATG | |
| | GATTCCAATTCCAAGGGAGGAG | |
| | GTGCCCTTTCAGCTATAGCTGC | |
| | CACCAACACTCCAGTAGCGTTT | |
| | ATTGCCACCGGAGAGCACATTC | |
| | AGAATTTCGAAAAGTTTTCAGG | |
| | AAGAGGATTTATCTCAAAACTTT | |
| | TAGGAATTGGTGATATAGAGGG | |
| | TCTTATGGAACATGTTCAGTCG | |
| | ATGAACTTGGATCAAGGTGATA | |
| | CTATCAAGAATTTCAAGGAAGG | |
| | AAAGTTTACTTTACAGGATTTTC | |
| | AAACGCAATTGAACAACATCAT | |
| | GAAGATGGGGCCACTGTCCAA | |
| | ACTCGCTCAAATGTTGCCTGGT | |
| | GGAATGGGACAATTGATGGGA | |
| | CAGGTTGGTGAAGAGGAGGCT | |
| | TCAAAGAGATTGAAGCGAATGA | |
| | TTTATATAATGGATTCAATGACG | |
| | AAGCAAGAGTTGTCAAGTGACG | |
| | GTAGATTGTTTATTGATCAGCCT | |
| | TCAAGGATGGTAAGAGTTGCTA | |
| | GAGGCTCTGGTACCTCTGTAAC | |
| | TGAGGTGGAGCTTGTTCTTTTA | |
| | CAGCAAAAGATGATGGCTCGTA | |
| | TGGCATTACAATCTAAGAATAT | |
| | GATGAGTGGGGCCGGCGGTCC | |
| | AGCAGGGATGGCTTCCAAAATG | |
| | AATCCAGCTAATATGAGAAGAG | |
| | CTATGCAACAAATGCAATCAAA | |
| | CCCAGGAATGATGGATAACATG | |
| | ATGAATATGTTTGGTGGAGCTG | |
| | GAGGAGCTGGAGGAGCTGGAA | |
| | TGCCGGATATGCAAGAAATGAT | |
| | GAAGCAAATGTCCAGTGGCCAA | |
| | ATGAAAATGCCCAGTCAACAGG | |
| | AAATGATGAGCATGATGAAACA | |
| | GTTTGGTATGGGCTAATAG | |
| | (SEQ ID NO: 16) | |
| SRP14 (Expressed under | ATGTCCACAACTACTAAGAAAA | MSTTTKKNKNRILIENHKQFL |
| pGPM1, and RPS3tt was | ACAAGAACAGGATCTTGATAGA | EEVSKTATLSVWNSKFSIKR |
| used as the terminator) | GAATCACAAACAGTTCCTGGAA | LSLEADPVEGTPEGIRDIPQ |
| (Prielhofer et al. 2017) | GAAGTTTCCAAAACAGCCACTT | GVETNSIIGNSVENDSKSHPI |
| | TATCAGTTTGGAACTCGAAATTT | LFRYTARHAKEKIPEVRISTT |
| | TCAATCAAACGACTGTCTCTAG | VDSEQLSTFWRDYVDILKGS |
| | AAGCAGATCCCGTGGAAGGGA | SQLKLQSETKKVSSKKSKAK |
| | CGCCTGAAGGAATCAGAGATAT | KKRGKGAW* (SEQ ID |
| | CCCACAAGGAGTAGAGACAAAC | NO: 10) |
| | TCTATAATAGGAAACAGCGTAG | |
| | AGAATGATTCAAAGTCACACCC | |
| | CATTTTATTCAGATATACAGCTA | |
| | GACACGCAAAGGAGAAAATACC | |
| | AGAGGTGCGAATTTCAACCACC | |
| | GTTGATTCAGAGCAGCTAAGCA | |
| | CCTTCTGGAGAGATTATGTGGA | |
| | CATATTGAAGGGAAGCTCCCAA | |
| | TTGAAACTGCAGTCAGAAACCA | |
| | AGAAAGTCAGTAGTAAGAAGAG | |
| | CAAGGCGAAGAAGAAGAGAGG | |
| | AAAGGGTGCATGGTAA (SEQ ID | |
| | NO: 17) | |
| non-coding RNA | ATGCAGCCTCTGATGAGTCCGT | |
| (Expressed under pTEF2, | GAGGACGAAACGAGTAAGCTC | |
| and IDPtt was used as | GTCAGGCTGTTATGGCGCATCC | |
| the terminator) | GGGGGAGGTAGTTACTTGACCT | |
| (Prielhofer et al. 2017). | TGATTCCTAATAGCTTACAACTG | |
| The non-coding RNA | AGGTGTCTCGTTCGATCCTGGC | |
| (underlined) was | GGTCCGCAATATTTTCCATACG | |
| overexpressed with a | AGTAATCTGTGGGGGAAGGCG | |
| hammerhead and HDV | AGCAATAAGACGTGCCACCGC | |
| ribozyme to remove | CCAAGGGGAGCAATCCAGCAG | |
| mRNA traits after RNA | GGAACACGTCCCGCAAGGAGG | |
| polymerase II | CGGGTGAGATAGCATCTCGTTG | |
| transcription | GTAATGGGCTGTTGGTGAACAA | |
| | AGTTTGACTATGTGAACCGGCT | |
| | ATTTACATTTTTGCTTTTT (SEQ | |
| | ID NO: 11) | |

Accordingly, the host cell, preferably a host cell of Pichia pastoris, of the invention may be engineered to overexpress one or more of SEQ ID NOs: 5-11 or a functional homolog thereof. The host cell, preferably a host cell of Pichia pastoris, of the invention may be engineered to overexpress one or more of SEQ ID NOs: 5-11. The host cell, preferably a host cell of Pichia pastoris, of the invention may also be engineered to overexpress SEQ ID NOs: 5-11 or a functional homolog thereof. The host cell, preferably a host cell of Pichia pastoris, of the invention may also be engineered to overexpress SEQ ID NOs: 5-11. Alternatively, the host cell, preferably a host cell of Pichia pastoris, of the invention may be engineered to overexpress one or more of SEQ ID NOs: 11-17 or a functional homolog thereof. The host cell, preferably a host cell of Pichia pastoris, of the invention may be engineered to overexpress one or more of SEQ ID NOs: 11-17. The host cell, preferably a host cell of Pichia pastoris, of the invention may be engineered to overexpress SEQ ID NOs: 11-17 or a functional homolog thereof. The host cell, preferably a host cell of Pichia pastoris, of the invention may be engineered to overexpress SEQ ID NOs: 11-17.

As used herein, “engineered” host cells are host cells which have been manipulated using genetic engineering, i.e. by human intervention. When a host cell is “engineered to overexpress” a given protein, the host cell is manipulated such that the host cell has an increased capability to express a protein or functional homologue in comparison to a non-engineered host cell thereby expression of a given protein, e.g. the fusion protein of the invention or additionally the one or more component(s) of the signal recognition particle (SRP) is increased compared to the host cell under the same condition prior to manipulation.

“Prior to engineering” when used in the context of host cells of the present invention means that such host cells are not engineered such that a polynucleotide encoding a protein such as the fusion protein of the invention and/or one or more components of the SRP or functional homologue thereof is overexpressed.

The term “recombining” as used herein means that a host cell of the present invention is equipped with a heterologous polynucleotide encoding a protein of interest, i.e., a host cell of the present invention is engineered to contain a heterologous polynucleotide encoding a protein of interest. This can be achieved, e.g., by transformation or transfection or any other suitable technique known in the art for the introduction of a polynucleotide into a host cell.

Overexpression can be achieved in any ways known to a skilled person in the art as will be described in the following in detail. In general, it can be achieved by increasing transcription and/or translation of the gene, e.g. by increasing the copy number of the gene or altering or modifying regulatory sequences or sites associated with expression of a gene. For example, overexpression can be achieved by introducing one or more copies of the polynucleotide encoding the protein, e.g.: the protein of interest and/or one or more component(s) of a signal recognition particle (SRP) or a functional homologue operably linked to regulatory sequences (e.g. a promoter) into the host cell or even into the genome resp. the chromosomes of the host cell by transformation. For example, the gene can be operably linked to a constitutive promoter and/or ubiquitous promoter or an inducible promoter in order to reach high expression levels. Such promoters can be endogenous promoters or recombinant promoters. Alternatively, it is possible to remove regulatory sequences such that expression becomes constitutive and/or increases when negatively regulatory sequences are removed. One can substitute the native promoter of a given gene with a heterologous promoter in the genome resp. chromosomes of the host cell which increases expression of the gene or leads to constitutive expression of the gene. For example, the promoter can be a strong constitutive promoter and/or strong ubiquitous promoter or a strong inducible promoter in order to reach higher expression levels than with the native promoter. In case a POI and/ or one or more component(s) of a signal recognition particle (SRP) foreign to the host cell is expressed the terms “expression” or “express” and “overexpression” or “overexpress” and “(over)expression” and “(over)express” are used interchangeably. For example, the protein of interest and/or one or more component(s) of a signal recognition particle (SRP) may be (over)expressed by more than 1%, 2%, 3%, 4%, 5%, 10%, 20%, 30%, 40%, 50%, 60%, 70%, 80%, 90%, 100%, 200%, or more than 300% by the host cell compared to the host cell prior to engineering and cultured under the same conditions. Accordingly, “overexpression” as used herein may relate to an increase of protein (e.g. one or more component(s) of a signal recognition particle (SRP) or a protein of interest) expression by a host cell in comparison to a host cell naturally expressing the protein but not engineered to overexpress said protein (control), and protein expression may be increased by more than any of 1%, 2%, 3%, 4%, 5%, 10%, 20%, 30%, 40%, 50%, 60%, 70%, 80%, 90%, 100%, 200%, or 300%. Furthermore “overexpression” as used herein may relate to the expression of a protein (e.g. a protein of interest) in comparison to a host cell not expressing the protein when not engineered to express the protein Using inducible promoters additionally makes it possible to increase the expression in the course of host cell cultivation. Furthermore, overexpression can also be achieved by, for example, modifying the chromosomal location of a particular gene, altering nucleic acid sequences adjacent to a particular gene such as a ribosome binding site or transcription terminator, modifying proteins (e.g., regulatory proteins, suppressors, enhancers, transcriptional activators and the like) involved in transcription of the gene and/or translation of the gene product, or any other conventional means of deregulating expression of a particular gene routine in the art (including but not limited to use of antisense nucleic acid molecules, for example, to block expression of repressor proteins or deleting or mutating the gene for a transcriptional factor which normally represses expression of the gene desired to be overexpressed. Prolonging the life of the mRNA may also improve the level of expression. For example, certain terminator regions may be used to extend the half-lives of mRNA (Yamanishi et al., Biosci. Biotechnol. Biochem. (2011) 75: 2234 and US 2013/0244243). If multiple copies of genes are included, the genes can either be located in plasmids of variable copy number or integrated and amplified in the chromosome. If the host cell does not comprise the gene product, it is possible to introduce the gene product into the host cell for expression. In this case, “overexpression” means expressing the gene product using any methods known to a skilled person in the art. Overexpression in comparison to a control as described above, e.g. the overexpression of one or more component(s) of a signal recognition particle (SRP) or a functional homologue thereof can be measured by methods known to a person skilled in the art, e.g.: by SDS Page, SDS Page/Western Plot, ELISA, or peptide map with subsequential mass spectrometry (proteomics). For example the contents of the one or more component(s) of a signal recognition particle (SRP) inside the cells can be measured and compared to the content in the control. Therefore the cells have to be disintegrated and the content is then measured in the cell lysate. Overexpression resp. expression of the protein of interest can be measured by e.g. measuring the content of the protein of interest in the supernatant of the respective host cell and the control and the contents are then compared. The content of the Protein of interest in the supernatant can be measured by methods known by a person skilled in the art, e.g. by SDS Page, SDS Page/Western Blot, ELISA, RP (reversed phase) HPLC, ion-exchange HPLC, and the like.

Those skilled in the art will find relevant instructions in Martin et al. (Bio/Technology 5, 137-146 (1987)), Guerrero et al. (Gene 138, 35-41 (1994)), Tsuchiya and Morinaga (Bio/Technology 6, 428-430 (1988)), Eikmanns et al. (Gene 102, 93-98 (1991)), EP 0 472 869, U.S. Pat. No. 4,601,893, Schwarzer and POhler (Bio/Technology 9, 84-87 (1991)), Reinscheid et al. (Applied and Environmental Microbiology 60, 126-132 (1994)), LaBarre et al. (Journal of Bacteriology 175, 1001- 1007 (1993)), WO 96/15246, Malumbres et al. (Gene 134, 15-24 (1993)), JP-A-10-229891, Jensen and Hammer (Biotechnology and Bioengineering 58, 191-195 (1998)) and Makrides (Microbiological Reviews 60, 512-538 (1996)), inter alia, and in well-known textbooks on genetics and molecular biology.

The nucleic acid or vector encoding the fusion protein of the invention and/or the polynucleotide encoding one or more component(s) of the SRP is/are preferably integrated into the genome, more preferred into a chromosome or a non-chromosomal genomic site of the host cell. The term “genome” generally refers to the whole hereditary information of an organism that is encoded in the DNA (or RNA for certain viral species). It may be present in the chromosome, on a plasmid or vector, or both. The vector or nucleic acid encoding the one or more component(s) of the SRP is preferably integrated into the genome of the host cell.

Polynucleotides encoding the fusion protein of the invention and/or the polynucleotide encoding one or more component(s) of the SRP may be recombined in the host cell by ligating the relevant genes each into one vector. It is possible to construct single vectors carrying the genes, or two separate vectors, one to carry the fusion protein of the invention and the other one the one or more component(s) of the SRP. These genes can be integrated into the host cell genome by transforming the host cell using such nucleic acids such as the nucleic acids of the invention or vectors described herein. In some embodiments, the gene encoding the fusion protein of the invention is integrated in the genome and the gene(s) encoding the one or more component(s) of the SRP is integrated in a plasmid or vector. In some embodiments, the gene(s) encoding the one or more components of the SRP is/are integrated in the genome and the gene(s) encoding the fusion protein(s) is/are integrated in a plasmid or vector. In some embodiments, the genes encoding the fusion protein of the invention and the polynucleotide encoding one or more component(s) of the SRP are integrated in the genome. In some embodiments, the genes encoding the fusion protein of the invention and/or the polynucleotide encoding one or more component(s) of the SRP are integrated in a plasmid or vector. If multiple genes encoding the fusion protein are used, some genes encoding the fusion protein can be integrated in the genome while others can be integrated in the same or different plasmids or vectors. If multiple genes encoding the one or more component(s) of the SRP are used, some of the genes encoding the one or more component(s) of the SRP can be integrated in the genome while others can be integrated in the same or different plasmids or vectors.

Overexpression of one or more component(s) of the SRP may also be achieved by exchanging the regulatory elements of the endogenous SRP protein(s) of the host cell by a regulatory element that leads to a higher transcription of the endogenous SRP protein(s) of the host cell.

### Methods of the Invention

The present invention further relates to a method of manufacturing a protein of interest in a eukaryotic host cell, comprising
(i) genetically engineering a recombinant host cell with the nucleic acid molecule of the invention or with the expression cassette of the invention, and optionally genetically engineering the recombinant host cell to overexpress one or more component(s) of a signal recognition particle (SRP);
(ii) culturing the genetically engineered host cell under conditions to express the nucleic acid molecule and optionally the one or more component(s) of the SRP, and to secrete the protein of interest upon cleavage of the secretion signal,
(iii) optionally isolating the protein of interest from the cell culture,
(iv) optionally purifying the protein of interest,
(v) optionally modifying the protein of interest, and
(vi) optionally formulating the protein of interest.

Step (i) of the method of manufacturing may also be understood as genetically engineering the recombinant host cell to overexpress a fusion protein of the invention and optionally one or more component(s) of the SRP (see also the relevant disclosure in the section “host cell of the invention”). When a host cell is “engineered to overexpress” a given protein, the host cell is manipulated such that the host cell has the capability to express, preferably overexpress the nucleic acid molecule of the invention and optionally the one or more component(s) of the SRP, thereby expression of a given protein, e.g. a fusion protein of the invention and optionally one or more component(s) of the SRP is increased compared to the host cell under the same condition prior to manipulation. In one embodiment, “engineered to overexpress” implies that a genetic alteration to a host cell is made in order to overexpress or increase expression of a protein, i.e. the cell is (intentionally) genetically engineered to overexpress such protein. Engineered to overexpress may include exchanging the promoter of the endogenous one or more component(s) of the SRP with a promoter leading to a higher transcription. Engineered to overexpress may include genetically engineering the host cell with a nucleic acid molecule, expression cassette or vector encoding the fusion protein described herein and/or the one or more component(s) of the SRP.

The present invention further relates to a method of producing a protein of interest by culturing a recombinant eukaryotic host cell of the invention under conditions to express the nucleic acid molecule of the invention and to secrete the protein of interest after cleavage of the secretion signal upon cleavage of the secretion signal, and isolating the protein of interest from the host cell culture upon cleavage of the secretion signal and optionally purifying and optionally modifying and optionally formulating the protein of interest.

“Prior to engineering” or “prior to manipulation” when used in the context of host cells disclosed herein means that such host cells are not engineered using a nucleic acid encoding the fusion protein of the invention. Said term thus also means that host cells do not overexpress a nucleic acid encoding the fusion protein of the invention and/or are not engineered to overexpress one or more component(s) of the SRP.

Procedures used to manipulate nucleic acid molecule sequences, e.g. coding for the fusion protein of the invention and/or the one or more component(s) of the SRP, the promoters, enhancers, leaders, etc., are well known to persons skilled in the art, e.g. described by J. Sambrook et al., Molecular Cloning: A Laboratory Manual (3rd edition), Cold Spring Harbor Laboratory, Cold Spring Harbor Laboratory Press, New York (2001).

A foreign or target polynucleotide such as the nucleic acid molecule of the invention can be inserted into the chromosome by various means, e.g., by homologous recombination or by using a hybrid recombinase that specifically targets sequences at the integration sites. The foreign or target polynucleotide described above is typically present in a vector (“inserting vector”). These vectors are typically circular and linearized before used for homologous recombination. As an alternative, the foreign or target polynucleotides may be DNA fragments joined by fusion PCR or synthetically constructed DNA fragments which are then recombined into the host cell. In addition to the homology arms, the vectors may also contain markers suitable for selection or screening, an origin of replication, and other elements. It is also possible to use heterologous recombination which results in random or non-targeted integration. Heterologous recombination refers to recombination between DNA molecules with significantly different sequences. Methods of recombinations are known in the art and for example described in Boer et al., Appl Microbiol Biotechnol (2007) 77: 513-523. One may also refer to Principles of Gene Manipulation and Genomics by Primrose and Twyman (7^{th} edition, Blackwell Publishing 2006) for genetic manipulation of yeast cells.

Nucleic acid molecules of the invention and/or the one or more component(s) of the SRP may also be present on a vector such as an expression vector. Such vectors are known in the art. In expression vectors, a promoter is placed upstream of the gene encoding the heterologous protein and regulates the expression of the gene. Multi-cloning vectors are especially useful due to their multi-cloning site. For expression, a promoter is generally placed upstream of the multi-cloning site. A vector for integration of the nucleic acid molecule encoding the fusion protein of the invention or the one or more component(s) of the SRP may be constructed either by first preparing a DNA construct containing the entire DNA sequence coding for the fusion protein of the invention or the one or more component(s) of the SRP subsequently inserting this construct into a suitable expression vector, or by sequentially inserting DNA fragments containing genetic information for the individual elements, such as the DNA binding domain, the activation domain, followed by ligation. As an alternative to restriction and ligation of fragments, recombination methods based on attachment sites (att) and recombination enzymes may be used to insert DNA sequences into a vector. Such methods are described, for example, by Landy (1989) Ann. Rev. Biochem. 58: 913-949; and are known to those of skill in the art.

Host cells according to the present invention can be obtained by introducing a vector or plasmid comprising the target polynucleotide sequences such as the nucleic acid molecule of the invention into the cells. Techniques for transfecting or transforming eukaryotic cells or transforming prokaryotic cells are well known in the art. These can include lipid vesicle mediated uptake, heat shock mediated uptake, electroporation, calcium phosphate mediated transfection (calcium phosphate/DNA co-precipitation), viral infection, particularly using modified viruses such as, for example, modified adenoviruses, microinjection and electroporation. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylyne glycol mediated uptake. The DNA can be single or double stranded, linear or circular, relaxed or supercoiled DNA. For various techniques for transfecting mammalian cells, see, for example, Keown et al. (1990) Processes in Enzymology 185: 527-537.

The phrase culturing the (genetically engineered) host cell under conditions to express the nucleic acid molecule of the invention and optionally overexpress the “one or more component(s) of the SRP” refers to maintaining and/or growing eukaryotic host cells under conditions (e.g., temperature, pressure, pH, induction, growth rate, medium, duration, feeding etc.) appropriate or sufficient to obtain production of the desired protein of interest or to overexpress the one or more component(s) of the SRP.

A host cell according to the invention obtained by engineering a host cell with the nucleic acid molecule of the invention or with the expression cassette of the invention, and optionally genetically engineering the host cell to overexpress one or more component(s) of the signal recognition particle (SRP) may preferably first be cultivated at conditions to grow efficiently to a large cell number without the burden of expressing a recombinant protein. When the cells are prepared for fusion protein expression, suitable cultivation conditions are selected and optimized to produce the fusion protein.

By way of example, using different promoters and/or copies and/or integration sites for the fusion protein(s) and the one or more component(s) of the SRP, the expression of the fusion protein(s) can be controlled with respect to time point and strength of induction in relation to the expression of the one or more component(s) of the SRP. For example, prior to induction of fusion protein, the one or more component(s) of the SRP may be first expressed. This has the advantage that the one or more component(s) of the SRP is already present at the beginning of translation of the fusion protein. Alternatively, the fusion protein and the one or more component(s) of the SRP can be induced at the same time.

An inducible promoter may be used that becomes activated as soon as an inductive stimulus is applied, to direct transcription of the gene under its control. Under growth conditions with an inductive stimulus, the cells usually grow more slowly than under normal conditions, but since the culture has already grown to a high cell number in the previous stage, the culture system as a whole produces a large amount of the recombinant protein. An inductive stimulus is preferably the addition of an appropriate agent (e.g. methanol for the AOX-promoter) or the depletion of an appropriate nutrient (e.g., methionine for the MET3-promoter). Also, the addition of ethanol, methylamine, cadmium or copper as well as heat or an osmotic pressure increasing agent can induce the expression depending on the promotors operably linked to the fusion protein and the one or more component(s) of the SRP.

It is preferred to cultivate the host cell(s) according to the invention in a bioreactor under optimized growth conditions to obtain a cell density of at least 1 g/L, preferably at least 10 g/L cell dry weight, more preferably at least 50 g/L cell dry weight. It is advantageous to achieve such yields of biomolecule production not only on a laboratory scale, but also on a pilot or industrial scale.

The host cell according to the invention may be tested for its expression/secretion capacity or yield by measuring the titer of the protein of interest in the supernatant of the cell culture or the cell homogenate of the cells after cell homogenisation by using standard tests, e.g. ELISA, activity assays, HPLC, Surface Plasmon Resonance (Biacore), Western Blot, capillary electrophoresis (Caliper) or SDS-Page.

Preferably, the host cells are cultivated in a minimal medium with a suitable carbon source, thereby further simplifying the isolation process significantly. By way of example, the minimal medium contains an utilizable carbon source (e.g. glucose, glycerol, ethanol or methanol), salts containing the macro elements (potassium, magnesium, calcium, ammonium, chloride, sulfate, phosphate) and trace elements (copper, iodide, manganese, molybdate, cobalt, zinc, and iron salts, and boric acid).

In the case of yeast cells, the cells may be transformed with one or more of the above-described expression vector(s), and cultured in conventional nutrient media modified as appropriate for inducing promoters, selecting transformants or amplifying the genes encoding the desired sequences. A number of minimal media suitable for the growth of yeast are known in the art. Any of these media may be supplemented as necessary with salts (such as sodium chloride, calcium, magnesium, and phosphate), buffers (such as HEPES, citric acid and phosphate buffer), nucleosides (such as adenosine and thymidine), trace elements, vitamins, and glucose or an equivalent energy source. Any other necessary supplements may also be included at appropriate concentrations that would be known to those skilled in the art. The culture conditions such as temperature, pH and the like are those previously used with the host cell selected for expression and are known to the ordinarily skilled artisan. Cell culture conditions for other type of host cells are also known and can be readily determined by the artisan. Descriptions of culture media for various microorganisms are for example contained in the handbook “Manual of Methods for General Bacteriology” of the American Society for Bacteriology (Washington D.C, USA, 1981).

Host cells can be cultured (e.g., maintained and/or grown) in liquid media and preferably are cultured, either continuously or intermittently, by conventional culturing methods such as test tube culture, shaking culture (e.g., rotary shaking culture, shake flask culture, etc.), aeration spinner culture, or fermentation. In some embodiments, cells are cultured in shake flasks or deep well plates. In yet other embodiments, cells are cultured in a bioreactor (e.g., in a bioreactor cultivation process). Cultivation processes include, but are not limited to, batch, fed-batch and continuous methods of cultivation. The terms “batch process” and “batch cultivation” refer to a closed system in which the composition of media, nutrients, supplemental additives and the like is set at the beginning of the cultivation and not subject to alteration during the cultivation; however, attempts may be made to control such factors as pH and oxygen concentration to prevent excess media acidification and/or cell death. The terms “fed-batch process” and “fed-batch cultivation” refer to a batch cultivation with the exception that one or more substrates or supplements are added (e.g., added in increments or continuously) as the cultivation progresses. The terms “continuous process” and “continuous cultivation” refer to a system in which a defined cultivation media is added continuously to a bioreactor and an equal amount of used or “conditioned” media is simultaneously removed, for example, for recovery of the desired product. A variety of such processes has been developed and is well-known in the art.

In some embodiments, host cells are cultured for about 12 to 24 hours, in other embodiments, host cells are cultured for about 24 to 36 hours, about 36 to 48 hours, about 48 to 72 hours, about 72 to 96 hours, about 96 to 120 hours, about 120 to 144 hours, or for a duration greater than 144 hours. In yet other embodiments, culturing is continued for a time sufficient to reach desirable production yields of POI.

The methods of the invention, e.g. the method of manufacturing a protein of interest or the method of increasing the secretion of a protein of interest, may further comprise a step of isolating the expressed POI. The POI is secreted from the cells and can be isolated and purified from the culture medium using state of the art techniques. During the process of secretion, the secretion signal is cleaved off. Secretion of the POI from the cells is generally preferred, since the products are recovered from the culture supernatant rather than from the complex mixture of proteins that results when cells are disrupted to release intracellular proteins. A protease inhibitor may be useful to inhibit proteolytic degradation during purification. The composition may be concentrated, filtered, dialyzed, etc., using methods known in the art. The cell culture after fermentation/cultivation can be centrifuged using a separator or a tube centrifuge to separate the cells from the culture supernatant. The supernatant can then be filtered of concentrated by using a tangential flow filtration.

An isolation and purification methods for obtaining the POI may be based on methods utilizing difference in solubility, such as salting out, solvent precipitation, heat precipitation, methods utilizing difference in molecular weight, such as size exclusion chromatography, ultrafiltration and gel electrophoresis, methods utilizing difference in electric charge, such as ion-exchange chromatography, methods utilizing specific affinity, such as affinity chromatography, methods utilizing difference in hydrophobicity, such as hydrophobic interaction chromatography and reverse phase high performance liquid chromatography, methods utilizing difference in isoelectric point, such as isoelectric focusing may be used and methods utilizing certain amino acids, such as IMAC (immobilized metal ion affinity chromatography). If the POI is expressed as inactive and soluble Inclusion Bodies the solubilized Inclusion Bodies need to be refolded.

The isolated and purified POI can be identified by conventional methods such as Western Blotting or specific assays for POI activity. The structure of the purified POI can be determined by amino acid analysis, amino-terminal peptide sequencing, primary structure analysis for example by mass spectrometry, RP-HPLC, ion exchange-HPLC, ELISA and the like. It is preferred that the POI is obtainable in large amounts and in a high purity level, thus meeting the necessary requirements for being used as an active ingredient in pharmaceutical compositions or as feed or food additive.

The term “isolated” as used herein means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature, e.g. cDNA made from mRNA; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

The term “modifying the protein of interest” is meant that the POI is chemically or enzymatically modified. There are many methods known in the art to modify proteins. Proteins can be coupled to carbohydrates or lipids. The POI may be PEGylated (the POI chemically coupled to polyethylenglycole) or HESylated (the POI is chemically coupled to hydroxyethyl starch) for half-life extension. The POI may also be coupled with other moieties such as affinity domains for e.g. human serum albumin for half life extension. The POI also may be treated by a protease or under hydrolytic conditions for cleavage to form the active ingredient from a pre-sequence or to cleave off a tag such as an affinity tag for purification. The POI may also be coupled to other moieties such as toxins, radioactive moieties or any other moiety. The POI may further be treated under conditions to form dimers, trimers and the like.

Additionally, the term “formulating the protein of interest” refers to bringing the POI to conditions, where the POI can be stored for a longer time. Many different methods known in the art are available to stabilize proteins. By exchanging the buffer in which the POI is existent after purification and/or modification, the POI can be brought under conditions, where it is more stable. Different buffer substances and additives, such as sucrose, mild detergents, stabilizer and the like, known in the art can be used. The POI can also be stabilized by lyophylization. For some POIs formulations can be done by formation of complexes of the POI with lipids or lipoproteins, such as polyplexes, and the like. Some protein may be co-formulated with other proteins.

The use of the secretion signal of the invention may increase the secretion of the protein of interest. Accordingly, the present invention also relates to a method of increasing the secretion of a protein of interest from a eukaryotic host cell, comprising expressing in said eukaryotic host cell a nucleic acid molecule of the invention and optionally genetically engineering the host cell to overexpress one or more component(s) of a signal recognition particle (SRP), thereby increasing the secretion of said protein of interest in comparison to a host cell expressing a fusion protein as defined herein but comprising a wild type Saccharomyces cerevisae α-mating factor secretion signal (such as SEQ ID NO: 4) instead of the secretion signal as defined in the nucleic acid molecule of the invention.

“Secretion” as used herein relates to the transfer of the protein of interest, which forms part of the fusion protein of the invention, out of the (recombinant) host cell. Thus, only the protein of interest and not the secretion signal is secreted. The signal peptide sequence is cleaved off in the endoplasmic reticulum and the MFα pro-sequence is cleaved off in the Golgi apparatus. Thus, if secretion is increased, only the secretion of the protein of interest is increased. The titer of the protein of interest in the supernatant of the cell culture can be determined using standard tests, e.g. ELISA, activity assays, HPLC, Surface Plasmon Resonance (Biacore), Western Blot, capillary electrophoresis (Caliper) or SDS-Page.

The method of increasing the secretion of a protein of interest from a eukaryotic host cell may further comprise engineering said host cell to incorporate an expression construct to express a nucleic acid molecule of the invention, and optionally genetically engineering the eukaryotic host cell to overexpress one or more component(s) of a signal recognition particle (SRP).

The method of increasing the secretion of a protein of interest from a eukaryotic host cell may further comprise culturing said host cell under conditions to express the nucleic acid molecule of the invention and optionally genetically engineering the host cell to overexpress the one or more component(s) of the SRP, and to secrete the protein of interest upon cleavage of the secretion signal.

The method of increasing the secretion of a protein of interest from a eukaryotic host cell may further comprise isolating the protein of interest from the cell culture.

The method of increasing the secretion of a protein of interest from a eukaryotic host cell may further comprise purifying the protein of interest.

The method of increasing the secretion of a protein of interest from a eukaryotic host cell may further comprise modifying the protein of interest.

The method of increasing the secretion of a protein of interest from a eukaryotic host cell may further comprise formulating the protein of interest.

In the method of increasing the secretion of a protein of interest from a eukaryotic host cell may further comprise, the nucleic acid molecule of the invention may be integrated in a chromosome of said host cell or contained in a vector or plasmid, which does not integrate into a chromosome of said host cell.

### Uses of the Invention

A person skilled in the art will readily acknowledge that the secretion signal described herein can be used to increase the secretion of a recombinant protein of interest. Accordingly, the present invention also relates to the use of the secretion signal as defined herein for increasing the secretion of a protein of interest from a eukaryotic host cell. As already described herein, the secretion signal may comprise from N- to C-terminus a signal peptide sequence originating from a KRE1 protein optionally followed by an α-mating factor (MFα) pro-sequence. Accordingly, the present invention also relates to the use of the secretion signal as defined herein for increasing the secretion of a protein of interest from a eukaryotic host cell, wherein the secretion signal comprises from N- to C-terminus a signal peptide sequence originating from a KRE1 protein followed by an α-mating factor (MFα) pro-sequence. The present invention also relates to the use of the secretion signal as defined herein for increasing the secretion of a protein of interest from a eukaryotic host cell, wherein the secretion signal comprises a signal peptide sequence originating from a KRE1 protein. As already described herein, the secretion signal may comprise from N- to C-terminus a signal peptide sequence originating from a SWP1 protein optionally followed by an α-mating factor (MFα) pro-sequence. Accordingly, the present invention also relates to the use of the secretion signal as defined herein for increasing the secretion of a protein of interest from a eukaryotic host cell, wherein the secretion signal comprises from N- to C-terminus a signal peptide sequence originating from a SWP1 protein followed by an α-mating factor (MFα) pro-sequence. The present invention also relates to the use of the secretion signal as defined herein for increasing the secretion of a protein of interest from a eukaryotic host cell, wherein the secretion signal comprises a signal peptide sequence originating from a SWP1 protein.

The secretion signal may increase secretion of the protein of interest from the eukaryotic host cell in comparison to a eukaryotic host cell expressing a fusion protein of the invention comprising the wild type Saccharomyces cerevisiae α-mating factor secretion signal (such as SEQ ID NO: 4) instead of the secretion signal as defined herein.

As the person skilled in the art will readily realize, the recombinant host cell of the invention is useful for the production of various proteins of interest, since they are efficiently secreted to the supernatant, thereby avoiding lysis of the host cells. Accordingly, the present invention further relates to the use of the recombinant host cell of the invention for manufacturing a protein of interest.

### Items

1. A nucleic acid molecule encoding a fusion protein comprising from N-terminus to C-terminus (a)
   a secretion signal, the secretion signal comprising
      (I) (i) a signal peptide sequence originating from a KRE1 protein or a signal peptide sequence originating from a SWP1 protein; and
         (ii) an α-mating factor (MFα) pro-sequence;
      or
      (II) a signal peptide sequence originating from a KRE1 protein or a signal peptide sequence originating from a SWP1 protein; and
   (b) a protein of interest.
2. The nucleic acid molecule of item 1, wherein the secretion signal increases secretion of said protein of interest from a eukaryotic host cell in comparison to said eukaryotic host cell expressing the nucleic acid molecule of item 1 but comprising a wild type Saccharomyces cerevisae α-mating factor secretion signal (such as SEQ ID NO: 4) instead of the secretion signal as defined in item 1.
3. The nucleic acid molecule of item 1 or 2, wherein the signal peptide sequence originating from a KRE1 protein comprises SEQ ID NO: 1 or a functional homolog thereof.
4. The nucleic acid molecule of item 1 or 2, wherein the signal peptide sequence originating from a SWP1 protein comprises SEQ ID NO: 2 or 52 or a functional homolog thereof.
5. The nucleic acid molecule of any one of the preceding items, wherein the MFα pro-sequence comprises SEQ ID NO: 3 or 53 or a functional homolog thereof, and/or wherein the MFα pro-sequence comprises Ser at a position corresponding to position 23 of SEQ ID NO: 53 and/or Glu at a position corresponding to position 64 of SEQ ID NO: 53.
6. The nucleic acid molecule of any one of items 1 to 5, wherein the protein of interest is selected from the group consisting of an antibody such as a chimeric, humanized or human antibody, or a bispecific antibody, or an antigen-binding antibody fragment such as Fab or F(ab)₂, single chain antibodies such as scFv, single domain antibodies such as VHH fragments of camelid or heavy chain antibodies or domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.
7. A secretion signal as defined in any one of items 1 to 6.
8. An expression cassette or a vector comprising the nucleic acid molecule of any one of items 1 to 6 and a promoter operably linked thereto.
9. A recombinant eukaryotic host cell comprising the nucleic acid molecule of any one of items 1 to 6, or the expression cassette or the vector of item 8, preferably
   (a) wherein the host cell is a fungal or yeast host cell, preferably a yeast host cell, selected from the group consisting of Komagataella phaffii (Pichia pastoris), Hansenula polymorpha, Saccharomyces cerevisiae, Kluyveromyces lactis, Yarrowia lipolytica, Pichia methanolica, Candida boidinii, Komagataella spp. and Schizosaccharomyces pombe, or a fungal host cell such as Trichoderma reesei or Aspergillus niger; and/or
   (b) wherein the host cell is engineered to overexpress one or more component(s) of a signal recognition particle (SRP).
10. A method of manufacturing a protein of interest in a eukaryotic host cell, comprising
   (i) genetically engineering the eukaryotic host cell with the nucleic acid molecule of any one of items 1 to 6 or with the expression cassette or vector of item 8, and optionally genetically engineering the eukaryotic host cell to overexpress one or more component(s) of a signal recognition particle (SRP);
   (ii) culturing the genetically engineered host cell under conditions to express the nucleic acid molecule and optionally to overexpress the one or more component(s) of the SRP, and to secrete the protein of interest upon cleavage of the secretion signal,
   (iii) optionally isolating the protein of interest from the cell culture,
   (iv) optionally purifying the protein of interest,
   (v) optionally modifying the protein of interest, and
   (vi) optionally formulating the protein of interest.
11. A method of increasing the secretion of a protein of interest from a eukaryotic host cell, comprising expressing in said eukaryotic host cell a nucleic acid molecule of any one of items 1 to 6 and optionally engineering the eukaryotic host cell to overexpress one or more component(s) of a signal recognition particle (SRP), thereby increasing the secretion of said protein of interest in comparison to said host cell expressing the nucleic acid molecule of items 1 to 6 except comprising a wild type Saccharomyces cerevisae α-mating factor secretion signal (such as SEQ ID NO: 4) instead of the secretion signal as defined in any of items 1 to 6.
12. The method of item 10 or 11,
   (a) wherein the method comprises
      (i) engineering said host cell to incorporate an expression construct to express a nucleic acid molecule of any one of items 1 to 6, and optionally genetically engineering the host cell to overexpress one or more component(s) of a signal recognition particle (SRP),
      (ii) culturing said host cell under conditions to express said nucleic acid molecule and optionally to overexpress the one or more component(s) of the SRP, and to secrete the protein of interest upon cleavage of the secretion signal,
      (iii) optionally isolating the protein of interest from the cell culture,
      (iv) optionally purifying the protein of interest,
      (v) optionally modifying the protein of interest, and
      (vi) optionally formulating the protein of interest; and/or
   (b) wherein the nucleic acid molecule is integrated in a chromosome of said host cell or contained in an expression cassette, vector or plasmid, which does not integrate into a chromosome of said host cell; and/or
   (c) wherein the eukaryotic host cell is a fungal or yeast host cell, preferably a fungal or yeast host cell, preferably a yeast host cell, selected from the group consisting of Komagataella phaffii (Pichia pastoris), Hansenula polymorpha, Saccharomyces cerevisiae, Saccharomyces paradoxus, Saccharomyces eubayanus, Saccharomyces kudriavzevii, Saccharomyces kluyveri, Saccharomyces uvarum, Kluyveromyces lactis, Yarrowia lipolytica, Pichia methanolica, Candida boidinii, Komagataella spp. and Schizosaccharomyces pombe, or a fungal host cell such as Trichoderma reesei or Aspergillus niger, and/or
   (d) wherein the protein of interest is selected from the group consisting of an antibody such as a chimeric, humanized or human antibody, or a bispecific antibody, or an antigen-binding antibody fragment such as Fab or F(ab)2, single chain antibodies such as scFv, single domain antibodies such as VHH fragments of camelid or heavy chain antibodies or domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.
13. Use of the secretion signal as defined in any one of items 1 to 7 for increasing the secretion of a protein of interest from a eukaryotic host cell, preferably wherein the secretion signal increases secretion of said protein of interest from the eukaryotic host cell in comparison to said eukaryotic host cell expressing the fusion protein as defined in item 1 comprising the wild type Saccharomyces cerevisae α-mating factor secretion signal (such as SEQ ID NO: 4) instead of the secretion signal as defined in item 1.
14. Use of the recombinant eukaryotic host cell of item 9 for manufacturing a protein of interest.
15. A method of producing a protein of interest by culturing the recombinant eukaryotic host cell of item 9 under conditions to express the nucleic acid molecule of any one of items 1 to 6 and to secrete the protein of interest upon cleavage of the secretion signal, and isolating the protein of interest from the host cell culture and optionally purifying and optionally modifying and optionally formulating the protein of interest.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” means plus or minus 20%, preferably plus or minus 10%, more preferably plus or minus 5%, most preferably plus or minus 1%.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

The examples below will demonstrate that the newly identified signal peptides in combination with the pro-sequence of Saccharomyces cerevisiae alpha-mating factor increase the titer (product per volume in mg/L) and the yield (product per biomass in mg/g biomass measured as wet cell weight) of secreted recombinant proteins compared to known secretion leaders including the commonly used S. cerevisiae alpha-mating factor pre-pro leader. As an example, the use of the novel signal peptides led to increased yield of different recombinant proteins and antibody derivatives in the yeast Pichia pastoris including single chain variable fragments, single domain antibodies, antigen binding fragments and easy to detect proteins (scR, VHH, SDZ-Fab, m-Cherry). The positive effect was shown in shaking cultures (conducted in 24 deep well plates) and in fed-batch bioreactor cultivations.

### Example 1: Selection of the Novel Signal Peptides

The most commonly used secretion signal yeasts including Pichia pastoris (syn. Komagataella spp) is the Saccharomyces cerevisiae alpha-mating factor (MFα) prepro-leader, i.e. the MFα secretion signal. Some recombinant proteins are inefficiently secreted with the MFα secretion signal and therefore only reach low production titers. Thus, there is the urgent need for novel signal peptides and secretion signals to increase secretion efficiency.

The initial and crucial step in secretion is the translocation of the recombinant protein into the endoplasmic reticulum (ER). This process is directed by an N-terminal cleavable secretion signal fused to the recombinant protein. For recombinant protein secretion, at least N-terminal cleavable signal peptide sequences are needed. These can be predicted from amino acid sequences with the most widely used program SignalP (Nielsen, 2017). Based on P. pastoris sequence data, SignalP 4.1 predicted 241 proteins having a cleavable signal peptide (SP) (Valli et al., 2016). However, there is no distinction between co- or post-translational SPs, i.e. the predicted SPs may act co- or post-translationally, and the amino acid sequence itself does not give information whether a predicted signal peptide sequence is able to secrete a recombinant fusion protein (i.e. a protein that differs from the natively secreted protein by this signal peptide sequence). To date, no sufficiently distinct features, concerning major physicochemical properties such as signal peptide sequence hydrophobicity, putative binding motifs or other sequence patterns, could be found that describe efficient signal peptides for co-translational or post-translational translocation of recombinant proteins (Janda et al., 2010; Pechmann et al., 2014; Massahi et al. (2016),.J Theor Biol., 408: 22-33.).

We used an in silico approach to select for novel efficient SPs. First, the hydrophobic mean was calculated using the hydropathy index normalized to the number of amino acids in the signal peptide (Kyte and Doolittle, 1982). Next, we searched for the most hydrophobic stretch of 8 amino acids in the SP and assigned a score with a maximum value of 1, representing the most hydrophobic stretch in the whole collection. Furthermore, we assessed the mean value of relative adaptiveness (Sharp and Li, 1987) of the codons 43 to 48 of the natively associated proteins and assigned a score with a maximum value of 1, representing the mean with the ‘slowest’ codons in this stretch. In other words, the score 0 was given when only optimal codons were present in this stretch. The two generated scores were summed up and candidates were selected which represented different rankings (Table 4:). Of the scored candidates, SP4 (first 18 amino acids of SWP1, PP7435_Chr1-0255) and SP14 (first 18 amino acids of KRE1, PP7435_Chr3-0933), represented the signal peptides with the highest and lowest sum score, respectively.

TABLE 4
Ranking of physicochemical properties of selected signal peptide sequence candidates
from Komagataella phaffii and MFα from Saccharomyces cerevisiae (MFα1)

| | | | Nr. of | codon | hydropathy | |
|---|---|---|---|---|---|---|
| | Gene | | amino | bias | stretch | COMBINED |
| Number | name | CBS7435 ORF | acids | score | (8aa) score | score |
| SP4* | SWP1 | PP7435_Chr1-0255 | 18 | 0.01 | 0.21 | 0.22 |
| SP10 | SLP1 | PP7435_Chr1-1234 | 16 | 0.06 | 0.57 | 0.64 |
| SP22 | | PP7435_Chr3-1213 | 20 | 0.09 | 0.63 | 0.72 |
| ScMFα | MFα1 | — | 20 | 0.41 | 0.38 | 0.79 |
| SP8 | GDT1 | PP7435_Chr2-0066 | 20 | 0.35 | 0.60 | 0.95 |
| SP9 | NCR1 | PP7435_Chr1-0478 | 18 | 0.25 | 0.80 | 1.05 |
| SP7* | OST3 | PP7435_Chr4-0360 | 18 | 0.85 | 0.30 | 1.15 |
| SP2 | GET1 | PP7435_Chr4-0582 | 16 | 0.81 | 0.54 | 1.35 |
| SP15 | | PP7435_Chr2-0965 | 24 | 0.63 | 0.75 | 1.38 |
| SP5* | WBP1 | PP7435_Chr2-0876 | 20 | 0.97 | 0.44 | 1.41 |
| SP11 | MSB2 | PP7435_Chr1-0283 | 20 | 0.95 | 0.51 | 1.45 |
| SP6* | OST1 | PP7435_Chr3-0451 | 16 | 0.74 | 0.90 | 1.64 |
| SP16 | | PP7435_Chr4-0694 | 18 | 0.84 | 0.81 | 1.65 |
| SP13 | PEP1 | PP7435_Chr2-0657 | 20 | 0.98 | 0.72 | 1.70 |
| SP18 | | PP7435_Chr4-0664 | 25 | 0.88 | 0.85 | 1.73 |
| SP3 | FET3 | PP7435_Chr2-0482 | 20 | 0.87 | 0.93 | 1.80 |
| SP14 | KRE1 | PP7435_Chr3-0933 | 18 | 0.92 | 1.00 | 1.92 |

### Example 2: Construction and Selection of P. pastoris Strains Secreting Recombinant Secretory Proteins Using the Novel Signal Peptide Sequences

### Construction of the Plasmids Carrying the Genes of Interest

P. pastoris CBS7435 mut^{s} variant (genome sequenced by Sturmberger et al. 2016) was used as host strain. The genes encoding the POIs (e.g. SDZ-Fab-LC, SDZ-Fab-HC, scR, VHH, and mCherry) were codon-optimized by Geneart or DNA2.0 (now ATUM) and obtained as synthetic DNA. A His6-tag was fused C-terminally to the scR and VHH genes for detection, while a FLAG-tag was added C-terminally of mCherry. The sequences of these proteins are shown in Table 2 of the description.

To construct expression vectors with the novel signal peptide sequences (see Table 4 and Table 7), the fragments selected for cloning were amplified by PCR (Q5® High-Fidelity DNA Polymerase, New England Biolabs). Genomic DNA from P. pastoris strain CBS7435 mut^{s}, synthetic genes or gBlocks (Integrated DNA Technologies) served as PCR templates. Amplified coding sequences were either cloned into the pPUZZLE-based expression plasmids pPM2dZ30 (described in WO2008/128701A2), or into the GoldenPiCS vectors (Prielhofer et al., 2017). The signal peptides (SP) were directly assembled with the promoters, terminators and product coding sequences into the expression plasmids.

The GoldenPiCS system (Prielhofer et al. 2017. BMC Systems Biol. doi: 10.1186/s12918-017-0492-3) requires the introduction of silent mutations in some coding sequences. Alternatively, gBlocks or synthetic codon-optimized genes were obtained from commercial providers (including Integrated DNA Technology IDT, Geneart, and ATUM). Amplified coding sequences were either cloned into the pPUZZLE-based expression plasmids pPM2aK21 or pPM2eH21, or the GoldenPiCS system (consisting of the backbones BB1, BB2 and BB3aZ/BB3aK/BB3eH/BB3rN). The gene fragments listed in Table 1 and Table 2 were introduced into the expression plasmids by using restriction enzymes. All promoters and terminators used to assemble expression cassettes in BB2 or BB3 backbones are described in Prielhofer et al. 2017 (BMC Systems Biol. doi: 10.1186/s12918-017-0492-3). pPM2aK21 and BB3aK allow integration into the 3′-AOX1 genomic region and contain the KanMX selection marker cassette for selection in E. coli and yeast. pPM2eH21 and BB3eH contain the 5′-ENO1 genome integration region and the HphMX selection marker cassette for selection on hygromycin. BB3rN contain the 5′-RGI1 genome integration region and the NatMX selection marker cassette for selection on nourseothricin. BB3aZ plasmids contain the Zeocin selection marker cassette and were linearized with Ascl to allow for integration into the 3′-AOX1 genomic region. All plasmids contain an origin of replication for E. coli (pUC19).

After electroporation (Example 3), transformants were selected on YPD (Yeast Extract, Peptone, Dextrose) plates containing the required antibiotics for the integrated selection markers (50 μg/mL Zeocin for Zeocin resistance marker with Sh ble gene, 500 μg/mL G418 for KanMX and 100 μg/mL Nourseothricin for NatMX) after incubation for 48h at 30° C. and singled under the same conditions.

The pPM2d_pGAP and pPM2d_pAOX expression plasmids are derivatives of the pPuzzle_ZeoR plasmid backbone described in WO2008/128701A2, consisting of the pUC19 bacterial origin of replication and the Zeocin antibiotic resistance cassette. Expression of the heterologous gene is mediated by the P. pastoris glyceraldehyde-3-phosphate dehydrogenase (GAP) or the alcohol oxidase (AOX) promoter, respectively, and the S. cerevisiae CYC1 transcription terminator. Some plasmids already contained the N-terminal S. cerevisiae alpha mating factor pre-pro leader sequence. After restriction digest with XhoI and BamHI (for scR) or EcoRV (for VHH), each gene was ligated into both plasmids pPM2d_pGAP and pPM2d_pAOX digested with XhoI and BamHI or EcoRV.

Plasmids were linearized using AvrII restriction enzyme (for pPM2d_pGAP) or PmeI restriction enzyme (for pPM2d_pAOX), respectively, prior to electroporation (using a standard transformation protocol as described in Gasser et al. 2013. Future Microbiol. 8(2): 191-208—see Example 3) into P. pastoris. Selection of positive transformants was performed on YPD plates (per liter: 10 g yeast extract, 20 g peptone, 20 g glucose, 20 g agar-agar) containing 50 μg/mL of Zeocin.

In the following examples, different heterologous proteins were used as reporters: the variable vH regions of a bivalent camelid antibody (VHH), a signal chain variable fragment antibody (scFv named scR), the antigen binding fragment of a human IgG (SDZ-Fab) and the fluorescent protein mCherry. The expression of the heterologous POIs was mediated by suitable P. pastoris promoters e.g. by the P. pastoris alcohol oxidase (AOX1) promoter (VHH, scR, SDZ-Fab-HC, mCherry) or the dihydroxyacetone synthase (DAS1) promoter (SDZ-Fab-LC) and S. cerevisiae CYC1 (VHH, scR, mCherry and SDZ-Fab-HC) or P. pastoris TDH1 (SDZ-Fab-LC) transcription terminators. Construction of the plasmids was done by using GoldenPiCS kit using Golden Gate Assembly as described in Prielhofer et al. (2017). The POI genes were amplified from vector DNA template (carrying the gene of interests) by PCR using primers that contained the SP sequences and each ligated into the BB1 plasmids using the restriction enzyme BsaI, thereby generating expression vectors harboring the tested signal peptides. For multimeric proteins such as the Fab, the individual expression vectors BB1_SDZ-Fab-HC and BB1_SDZ-Fab-LC were then assembled into the BB2 plasmid by using the restriction enzyme BpiI, resulting in BB2_ab_pA0X1_SDz-Fab-HC-CYC1tt and BB2_bc_pDAS1_SDZ-Fab-LC-TDH1tt plasmids. These BB2 plasmids were finally combined to generate the BB3aZ_SDZ-Fab plasmid which comprises expression cassettes for both the HC and the LC of the Fab fragment. For VHH and scR, BB1 plasmids were directly assembled with BB3aZ plasmids, which resulted in BB3aZ_VHH and BB3aZ_scR, respectively. The novel signal peptides were either added as part of the 5′ primer sequence, by using fusion PCR or by Golden Gate assembly after amplifying them by PCR. After sequence verification, the expression cassettes for all proteins were combined onto one vector by using the compatible restriction enzymes, BpiI and BsaI. Coding DNA sequences of the recombinant proteins are given in Table 2. For secretion, either the newly identified signal peptides alone (Sequences see Table 7), the newly identified signal peptides in combination with the pro-sequence of S. cerevisiae alpha mating factor (MFα) secretion leader, SEQ ID NO: 3, or the entire secretion signal of S. cerevisiae MFα, SEQ ID NO: 4, were used.

### Example 3: Generation of the P. pastoris Strains Producing Recombinant Secretory Proteins With the Novel Signal Peptide Sequences

POI expression plasmids (up to 3 μg) were linearized by AscI prior to electroporation into P. pastoris. For this purpose the P. pastoris strain was made electro competent. The strain was inoculated into 100 mL YPD media (main culture) for 16-20 hours (25° C.; 180 rpm) and harvested at an optical density (OD₆₀₀) from 1.8-3 by centrifugation (5 min; 1500 g; 4° C.) in two 50 mL falcon tubes. The cell pellet was resuspended in 10 mL YPD+20 mM HEPES+25 mM DTT and incubated (30 min; 25° C.; 180 rpm). After the incubation period the falcon tubes were filled with 40 mL ice cold sterile distilled water and centrifuged (5 min; 1500 g; 4° C.) (Eppendorf AG, Germany). The cell pellet was resuspended in ice cold sterile 1 mM HEPES buffer (pH 8) and centrifuged (30 min; 25° C.; 180 rpm). The cell pellet was resuspended in 45 mL ice cold 1 M sorbitol and centrifuged (30 min; 25° C.; 180 rpm). The pellet was resuspended in 500 μL ice cold 1M sorbitol and 80 μL aliquoted into ice cold 1.5 mL Eppendorf tubes. The aliquoted electro competent cells were kept at −80° C. until used.

The electroporation was performed at 2 kV for 4 milliseconds (Gene Pulser, Bio-Rad Laboratories, Inc, USA). After transformation the electroporated cells were suspended in 1 mL YPD media and regenerated for 1.5 h to 3 h on 30° C. shaking at 650 rpm on a thermoshaker (Eppendorf AG, Germany). Later, 20 μL and 200 μL of the cell suspension was plated on YPD plates (per liter: 10 g yeast extract, 20 g peptone, 20 g glucose, 20 g agar-agar) containing 50 μg/mL Zeocin (CBS7435 mutS background) or 50 μg/mL Zeocin and 100 μg/mL nourseothricin (CBS7435 mutS co-expressing SRP, see Example 5) for selection and incubated on 30° C. for 48 hours. The colonies that appeared were re-streaked onto fresh YPD plates containing the appropriate antibiotics.

### Example 4: Small Scale Cultivation (Screening) of the P. pastoris Strains Producing Recombinant Secretory Proteins With the Novel Signal Peptide Sequences

### Cultivation

24 deep well plates (DWP) sealed with an air permeable membrane were used for the screening. Single colonies (20 colonies from each transformation) were picked from transformation plates for the pre-culture and used to inoculate 2 mL of YPD with the appropriate antibiotics based on the antibiotic resistance used for selection. Pre-cultures were grown for ca. 24 h at 25° C. and 280 rpm in 24-DWP and subsequently used to inoculate 2 mL of synthetic screening medium ASMv6 (per liter: 22.0 g Citric acid monohydrate, 6.3 g (NH₄)₂ HPO₄, 0.49 g MgSO₄*7H₂O, 2.64 g KCl, 0.054 g CaCl₂*2H₂O, 1.47 mL PTMO trace salts stock solution (per liter: 6.0 g CuSO₄*5H₂O, 0.08 g NaI, 3.36 g MnSO₄*H₂O, 0.2 g Na₂MoO₄*2H₂O, 0.02 g H₃BO₃, 0.82 g CoCl₂*2H₂O, 20.0 g ZnCl₂, 65.0 g FeSO₄*7H₂O and 5.0 ml H₂SO₄ (95%-98%)), 4 mg Biotin; pH was set to 6.5 with KOH (solid)) containing 25 g L⁻¹ polysaccharide and 0.35% of glucose-releasing enzyme solution (Enpresso) to a starting-OD₆₀₀ of 8 (t=0 h). The main culture lasted for 48 h and methanol was added four times for induction (0.5% at t=4 h, 1% at t=20 h, t=28 h and t=44 h each).

### Harvest and Analysis

After 48 h, 1 mL of each culture was removed and centrifuged in a pre-weighted Eppendorf tube. The wet cell weight (VVCVV) was determined by weighting the Eppendorf tube with the cell pellet and calculated as follows: Weight (full)−Weight (empty)=Wet cell weight (WCVV) (g/L). The supernatant was used to quantify the recombinant secreted protein concentration using microfluidic capillary electrophoresis (mCE), ELISA or fluorescence spectroscopy as described below. Out of this data the yield was calculated: Yield (μg/mg)=Protein concentration/Wet cell weight. The volumetric titer (also referred to as “titer”) is the content of the protein of interest (POI) in the supernatant of the cultivations as described in Examples 4 and 6 in g/L or mg/L and the like. Only clones having one copy of gene of interest were included in the analysis. Gene copy number of clones selected for bioreactor cultivation was determined by qPCR (Example 4). Titer and yield fold changes are given respective to the reference clone that has 1 copy of gene of interest that is secreted by using the S. cerevisiae MFα secretion signal (SEQ ID NO: 4).

Quantification by Microfluidic Capillary Electrophoresis (mCE)

The ‘LabChip GX/GXII System’ (PerkinElmer) was used for quantitative analysis of secreted protein titer in culture supernatants. The consumables ‘Protein Express Lab Chip’ (760499, PerkinElmer) and ‘Protein Express Reagent Kit’ (CLS960008, PerkinElmer) were used. Briefly, 6 μL of culture supernatant were mixed with 21 μL of non-reducing sample buffer. This mixture was denatured at 100 ° C. for 5 min, briefly centrifuged and further mixed with 105 μL water (Milli-Q® or equivalent). Samples were then centrifuged at 1200 g for 2 min and applied to the instrument. The fluorescently labelled samples were analyzed according to protein size in the instrument, using an electrophoretic system based on microfluidics. Internal standards enabled approximate allocations to size in kDa and approximate concentrations of detected signals.

### Quantification of Fab by ELISA

Quantification of intact Fab by ELISA was done using anti-human IgG antibody (ab7497, Abcam) as coating antibody and a goat anti-human IgG (Fab specific)—alkaline phosphatase conjugated antibody (Sigma A8542) as detection antibody. Human Fab/Kappa, IgG fragment (Bethyl P80-115) was used as standard with a starting concentration of 100 ng/mL, supernatant samples are diluted accordingly. Detection was done with pNPP (Sigma S0942). Coating-, Dilution- and Washing buffer were based on PBS (2 mM KH₂PO₄, 10 mM Na₂HPO₄.2 H₂O, 2.7 mM g KCl, 8 mM NaCl, pH 7.4) and completed with BSA (1% (w/v)) and/or Tween20 (0.1% (v/v)) accordingly.

### Quantification by Fluorescence Spectroscopy

Secreted mCherry fluorescence was directly measured by transferring 100 μL of each screening supernatant into a FluoroNunc™/LumiNunc™ 96-Well Plate (ref. 10366281, Thermo Fischer Scientific, Waltham, USA). An mCherry standard (ref. TP790040, OriGene Technologies, Herford, Germany) was diluted in the same plate, in PBSG (1× PBS in 10% glycerol) to get a standard curve from 0 to 80 ng·μL−1. The plate was loaded in the Infinite M200 device, and controlled by the i-control v. 1.6 software. Prior measurement, samples were shaken in a linear mode for 5 s, with an amplitude of 1 mm. The measurement, ran in a fluorescence top reading mode, consisted in 25 flashes of 20 μs each at 587 nm (bandwidth of 9 nm) and the emission was read, without lag time, at 640 nm (bandwidth of 20 nm).

Determination of Gene Copy Numbers by Quantitative Real-Time PCR (qPCR)

Cell pellets from 1 mL culture were first prepared for lysis with lyticase (5 U/μL with the addition of 50 mM 2-mercaptoethanol) and genomic DNA was then further extracted with the DNeasy® Blood & Tissue Kit (Qiagen). The qPCR reaction was made up of 0.25 μL of forward and 0.25 μL of reverse primer (Table 5), 5 μL of 2× SensiMix SYBR Hi-ROX Kit (Bioline), 3.5 μL of nuclease-free water and 1 μL of the sample. The qPCRs were performed under the following conditions: 10 min hot start at 95° C. followed by 45 cycles of 15 s at 95° C., 20 s at 60° C., and 15 s at 72° C. on a Rotorgene 6000 (Qiagen). The GCNs were determined by normalizing the fluorescence signal of the respective sample to the chosen control sample by using the Rotor-Gene software comparative quantification method. This ratio was further normalized to the ACT1 signal of the same sample to compensate for initial concentration differences.

TABLE 5
List of primers required for quantitative PCR analysis. Notation of DNA
sequences is from 5′ to 3′

| gene name | forward primer | reverse primer |
|---|---|---|
| SRP14 | AGACACGCAAAGGAGAAAA (SEQ ID | GTCCACATAATCTCTCCAGAAG (SEQ |
| | NO: 30) | ID NO: 31) |
| SRP21 | GGGGAGCCACCAGTATTTT (SEQ ID | CCACCTTTACCACCTTTCTTT (SEQ |
| | NO: 32) | ID NO: 33) |
| SEC65 | TTCCTTTGCATTCGCCATT (SEQ ID | TTCTTCTGTTTCGGAGCTTTG (SEQ |
| | NO: 34) | ID NO: 35) |
| SPR54 | AAGATGATGGCTCGTATGG (SEQ ID | TCCTGGGTTTGATTGCATTT (SEQ ID |
| | NO: 36) | NO: 37) |
| SRP68 | CAACTACGTGAGTCAACCAA (SEQ | AGCGGAACAATCCAAACAA (SEQ ID |
| | ID NO: 38) | NO: 39) |
| SRP72 | TGCCTTCAAAACCCTCAATC (SEQ | GGTCGTGGTAGTGTTATTGT (SEQ ID |
| | ID NO: 40) | NO: 41) |
| scpRNA | GGGAAGGCGAGCAATAAG (SEQ ID | ACCAACAGCCCATTACCA (SEQ ID |
| | NO: 42) | NO: 43) |
| SCR | AAGCCTGGTAAGCCTCCAAAGT | TCCTCAGCTTGAACACCACCAAT |
| | (SEQ ID NO: 44) | (SEQ ID NO: 45) |
| VHH | TGTAACGTGAATGTCGGATTTG | TAGTGATGGTGGTGGTGATG (SEQ |
| | (SEQ ID NO: 46) | ID NO: 47) |
| SDZ-Fab- | TACTGCTGCTTTGGGTTGTTTGGT | AAGGGACAGTAACAACAGAGGACA |
| HC | (SEQ ID NO: 48) | (SEQ ID NO: 49) |
| SDZ-Fab- | GATGAACAATTGAAGTCTGGTAC | GAGTAACTTCACAAGCGTAAACC |
| LC | (SEQ ID NO: 50) | (SEQ ID NO: 51) |
| mCherry | CATCAAGTTGGACATCACCTCC | CACCCTTGTACAGCTCGTCC (SEQ |
| | (SEQ ID NO: 67) | ID NO: 68) |

### Example 5: Generation of a Signal Recognition Particle (SRP) Overexpression P. pastoris Strain

The inventors further decided to test the SPs in an SRP overexpressing strain to test whether the secretion pathway would be prepared to cope with a high load of recombinant protein fused to the novel SPs.

To generate the SRP expression plasmid 236_BB3rN, expression cassettes for all seven subunits of the SRP (6 protein subunits and 1 non-coding RNA) were assembled in one single plasmid using Golden Gate-based cloning as described in Prielhofer et al. (2017) to ensure equal gene copy numbers. The genes for all subunits were amplified by PCR from the genome of CBS7435 and cloned into BB1 plasmids, which were then assembled with the respective promoters and terminators in BB2 plasmids. Used promoters and transcription terminators for expression of the SRP subunits are given in Table 6. The non-coding RNA was overexpressed with a hammerhead and HDV ribozyme to remove mRNA traits after RNA polymerase II transcription.

TABLE 6
Promoters and terminators used for the overexpression
of SRP subunits for the SRP background strain.

| | Chromosomal | | |
|---|---|---|---|
| SRP subunit | Location | Promoter * | Terminator * |
| SRP68 | PP7435_Chr1-0901 | pADH2 | RPL2aTT |
| SRP72 | PP7435_Chr1-0988 | pPOR1 | RPP1bTT |
| SRP9-21 | PP7435_Chr3-0697 | pPDC1 | RPS25aTT |
| SEC65 | PP7435_Chr3-0671 | pRPP1b | RPS17bTT |
| SRP54 | PP7435_Chr4-0671 | pFBA1-1 | RPS2TT |
| SRP14 | PP7435_Chr4-0320 | pGPM1 | RPS3TT |
| non-coding RNA | PP7435_Chr1-2610 | pTEF2 | IDP1TT |

* Reference for promoter and terminator sequences: Prielhofer, R., Barrero, J.J., Steuer, S. et al. GoldenPiCS: a Golden Gate-derived modular cloning system for applied synthetic biology in the yeast Pichia pastoris. BMC Syst Biol 11, 123 (2017). https://doi.org/10.1186/s12918-017-0492-3

To create an SRP overexpressing background strain, CBS7435 mutS was transformed (Example 3) with the plasmid 236_BB3rN carrying all seven SRP subunits. 20 transformants and 4 control strains were cultivated according to the 24 deep well plate screening procedure (Example 4). The wet cell weights of the transformed and 4 untransformed clones did not show any significant differences. Next, gene copy number (GCN) analysis (Example 4) of the 7 SRP protein components was performed on three randomly selected clones (clone #3, #7 and #10). For #3 and #10, the GCN of the SRP genes was determined to be 2, indicating the integration of 1 overexpression cassette into the genome. Clone #7 (named CBS7435 mutS SRP#7) had 3 copies of the genes, indicating the integration of 2 overexpression cassettes. This clone was selected as the background SRP overexpressing P. pastoris strain for the expression of heterologous proteins.

### Example 6: Bioreactor Cultivations of the P. pastoris Strains Producing Recombinant Secretory Proteins With the Novel Signal Peptide Sequences

Clones expressing the protein of interest (POI, e.g. VHH, scR, SDZ-Fab, mCherry) by using the novel signal peptide sequences as well as control strains using the full-length MFα secretion signal (SEQ ID NO: 4) (Example 2) were selected after small scale screening cultivations (Example 4). The selected clones were further evaluated in larger cultivation volumes by fed batch bioreactor cultivations.

### Pre-Culture

Clones were inoculated into wide-necked, baffled, covered 300 mL shake flasks filled with 50 mL of YPhyG (per liter: 20.0 g Phytone-Peptone, 10.0 g Bacto-Yeast Extract, 20.0 g glycerol) and shaken at 110 rpm at 28° C. overnight (pre-culture 1). Pre-culture 2 (200 mL YPhyG in a 2000 mL wide-necked, baffled, covered shake flask) was inoculated from pre-culture 1 in a way that the OD₆₀₀ (optical density measured at 600 nm) reached approximately 20 (measured against YPhyG media) in late afternoon. Incubation of pre-culture 2 was performed at 110 rpm at 28° C., as well.

### Production-Batch Phase

The fermentations (bioreactor cultivations; all phases) were carried out in fully instrumented and controllable Infors Multifors 1L-reactors (750 mL working volume). All fermenters filled with 400 mL BSM-media (per liter: 13.5 mL H₃PO₄ (85%), 0.5 g CaCl.2H₂O, 7.5 g MgSO₄.7H₂O, 9.0 g K₂SO₄, 2.0 g KOH, 40 g glycerol, 0.25 g NaCl, 0.1 mL antifoam (Glanapon 2000), 4.35 mL PTM1 (per liter: 0.2 g biotin, 6.0 g CuSO₄.5H₂O, 0.09 g KI, 3.0 g MnSO₄H₂O, 0.2 g Na₂MoO₄.2H₂O, 0.02 g H₃BO₃, 0.5 g CoCl₂, 42.2 g ZnSO₄.7H₂O, 65.0 g Fe(II)SO₄.7H₂O, 5 mL H₂SO₄) with a pH of approximately 5.5 were individually inoculated from pre-culture 2 to an OD₆₀₀ of 2.0. P. pastoris was grown on glycerol to produce biomass and the culture was subsequently subjected to glycerol feeding (60% w/w+12 ml/L PTM1) followed by mixed methanol/glycerol feeding and subsequent methanol feeding. Ammonia solution (25%) was used for pH control.

In the initial batch phase, the temperature was set to 28° C. Over the period of the last hour before initiating the production phase it was decreased to 24° C. and kept at this level throughout the remaining process, while the pH dropped to 5.0 and was kept at this level. Oxygen saturation was set to 30% throughout the whole process (cascade control: stirrer, flow, oxygen supplementation). Stirring was applied between 700 and 1200 rpm and a flow range (air) of 1.0-2.0 L min⁻¹ was chosen.

During the batch phase, biomass was generated (μ˜0.30 h⁻¹) up to a wet cell weight (WCVV) of approximately 110-120 g L⁻¹. The classical batch phase (biomass generation) would last about 14 hours.

### Production-Fed Batch Phase

Glycerol was fed with a rate defined by the equation 2.6+0.3*t (g glycerol (60%) /h), wherein t=time in hours so a total of 30 g glycerol (60%) was supplemented within 8 hours. The first sampling point was selected to be 20 hours. In the next 18 hours (from process time 20 to 38 hours), a mixed feed of glycerol/methanol was applied:
glycerol feed rate defined by the equation: 2.5+0.13*t (g glycerol (60%)/h), supplying 66 g glycerol (60%)
methanol feed rate defined by the equation: 0.72+0.05*t (g methanol (100%)/h), adding 21 g of methanol

During the next approx. 72.5 hours (from process time 38 to approx. 110.5 hours) methanol was fed by the equation 2.2+0.016*t (g methanol (100%)/h), resulting in a supply of approx. 223 g of methanol.

### Sampling and Analysis

Samples were taken at indicated time-points with the following procedure: the first 3 mL of sampled fermentation broth (with a syringe) were discarded. 1×1 mL of the freshly taken sample (3-5 mL) were transferred into 1.5 mL centrifugation tubes and spun for 5 minutes at 13200 rpm (16100 g). Supernatants were diligently transferred into separate vials and immediately frozen, along with the corresponding wet pellet fractions. To determine the WCW, 1 mL of fermentation broth was centrifuged in a tared Eppendorf vial at 13200 rpm (16100 g) for 5 minutes and the resulting supernatant was accurately removed. The vial was weighed (accuracy 0.1 mg), and the tare of the empty vial was subtracted to obtain wet cell weights. Quantification of the proteins was performed as explained in Example 4.

### Example 7: Screening Results of the P. pastoris Strains Producing Recombinant Secretory Proteins With The Novel Signal Peptide Sequences

Transformation of P. pastoris strains was performed as described in Example 3. The secretion improvement obtained with the novel SPs (Example 4) is measured by titer and yield fold-change values (also named FC titer or titer FC resp. FC yield or yield FC) that refer to the respective control clones secreting the POI using the MFα secretion signal (SEQ ID NO 4) (Example 1) in the same strain background (CBS7435 mutS WT or CBS7435 mutS SRP#7, Example 5). Titer fold change is understood as the quotient of the titer of the respective fermentation or small scale cultivation divided by the titer of the control. Yield fold change is understood as the quotient of the yield of the respective fermentation or small scale cultivation divided by the yield of the control.

Screening of the performances of the novel signal peptides (SPs) for the production of proteins of interest (POIs) in comparison to the MFα secretion signal was performed as described in Example 4.

The results of the screenings of the SP candidates shown in Table 4 and Table 7 using VHH as reporter protein for secretion are shown in Table 7.

TABLE 7
creening results obtained by using the candidate SPs alone as secretion
signal without an MFα-pro sequence for the secretion of VHH or mCherry as
reporter POIs compared to using the MFα secretion signal in the SRP
background (CBS7435 mutS SRP#7). Average fold changes of titers of up to
20 clones per construct are shown.

| Name | POI | FC titer | Sequence |
|---|---|---|---|
| SP4* | VHH | 1.34 | MKLISVGIVTTLLTLASC (SEQ ID NO: 2) |
| SP10 | VHH | 0.86 | MLVAWFLLLLVSSCIC (SEQ ID NO: 56) |
| SP22 | VHH | 0.59 | MKFAISTLLIILQAAAVFA (SEQ ID NO: |
| | | | 57) |
| SP8 | VHH | 0.59 | MKFGLGSLGLAVALIPIASA (SEQ ID NO: |
| | | | 58) |
| SP9 | VHH | 0.61 | MIILLPLLFLFVAGLVQA (SEQ ID NO: 59) |
| SP2 | VHH | incorrect cleavage | MDPFSILLTLTLIILA (SEQ ID NO: 60) |
| SP15 | VHH | 0.55 | MRLSYECLFSVFLVLAYHLKGTKA (SEQ |
| | | | ID NO: 61) |
| SP11 | VHH | 0.65 | MINLNSFLILTVTLLSPALA (SEQ ID NO: |
| | | | 62) |
| SP16 | VHH | 0.53 | MQLQYLAVLCALLLNVQS (SEQ ID NO: |
| | | | 63) |
| SP13 | VHH | 0.47 | MWIERNLIASILLFSTSAYA (SEQ ID NO: |
| | | | 64) |
| SP18 | VHH | incorrect cleavage | MNISTASKISRLLQLVIALISLVLT (SEQ ID |
| | | | NO: 65) |
| SP3 | VHH | 0.22 | MFVFEPVLLAVLVASTCVTA (SEQ ID |
| | | | NO: 65) |
| SP14 | VHH | 1.04 | MLNKLFIAILIVITAVIG (SEQ ID NO: 1) |
| SP14 | mCherry | 1.40 | MLNKLFIAILIVITAVIG (SEQ ID NO: 1) |

As can be seen in Table 7, SP4 and SP14 represent highly effective SPs when used as sole secretion signal for secretion of POIs, exceeding secreted product titers in comparison to the MFα secretion signal by approximately 1.3-1.4-fold.

Next, the inventors decided to test if there is a further impact by fusing the novel SPs to a pro-sequence such as the MFα pro-sequence.

A major impact was observed when the signal peptides were fused to the MFα-pro sequence (see Table 8). From these screenings we concluded that addition of a pro-sequence such as the MFα-pro sequence is even more beneficial for recombinant protein production.

TABLE 8
Screening results obtained by using the SPs, SP4 and SP14
fused to the MFα-pro sequence for the secretion of VHH,
scR or SDZ-Fab as reporter POIs compared to using the MFα
secretion signal in either CBS7435 mutS WT (WT) or CBS7435
mutS SRP#7 (SRP) background. Average fold changes of
titers of up to 20 clones per construct are shown.

| Background | Protein | Pre | Pro | Titer FC | Yield FC |
|---|---|---|---|---|---|
| SRP | VHH | SP4 | MFα-pro | 1.9 | 1.9 |
| SRP | VHH | SP14 | MFα-pro | 1.7 | 1.8 |
| WT | VHH | SP14 | MFα-pro | 1.5 | 1.5 |
| SRP | scR | SP4 | MFα-pro | 1.4 | 1.5 |
| SRP | scR | SP14 | MFα-pro | 1.3 | 1.3 |
| WT | scR | SP4 | MFα-pro | 1.2 | 1.2 |
| WT | SDZ-Fab | SP14 | MFα-pro | 1.7 | 1.7 |

Fusion of the different signal peptides (SPs) to other Pichia pastoris (Komagataella phaffii) derived pro-sequences resulted in lower or even no secretion of the POI compared to the combination with the MFα-pro sequence (see Table 9).

TABLE 9
Screening results obtained by using the SPs, SP4 and SP14
fused to different Pichia pastoris (Komagataella phaffii)
pro sequences for the secretion of VHH, scR or SDZ-Fab as
reporter POIs compared to using the MFα secretion
signal (SEQ ID NO: 4) in either CBS7435 mutS WT (WT) or
CBS7435 mutS SRP#7 (SRP) background. Average fold changes
of titers of up to 20 clones per construct are shown.

| | | | | Titer | Yield |
|---|---|---|---|---|---|
| Background | Protein | Pre | Pro | FC | FC |
| WT | VHH | SP4 | SEQ ID NO: 69 | 0.0 | 0.0 |
| WT | VHH | SP14 | SEQ ID NO: 69 | 0.0 | 0.0 |
| WT | VHH | SP4 | SEQ ID NO: 70 | 0.7 | 0.7 |
| WT | VHH | SP14 | SEQ ID NO: 70 | 0.4 | 0.5 |
| WT | VHH | SP4 | SEQ ID NO: 71 | 0.6 | 0.6 |
| WT | VHH | SP14 | SEQ ID NO: 71 | 0.8 | 0.8 |
| WT | VHH | SP4 | SEQ ID NO: 72 | 0.2 | 0.2 |
| WT | VHH | SP14 | SEQ ID NO: 72 | 0.0 | 0.0 |
| WT | VHH | SP4 | SEQ ID NO: 73 | 0.4 | 0.4 |
| WT | VHH | SP14 | SEQ ID NO: 73 | 0.8 | 0.8 |
| WT | scR | SP4 | SEQ ID NO: 71 | 0.6 | 0.6 |
| WT | SDZ-Fab | SP14 | SEQ ID NO: 71 | 0.4 | 0.4 |
| WT | SDZ-Fab | SP14 | SEQ ID NO: 73 | 0.0 | 0.0 |

### Example 8. Fed Batch Cultivations of P. pastoris Strains Producing Recombinant Secretory Proteins With the Novel Signal Peptide Sequences

Bioreactor cultivations were performed as explained in Example 6. First, single copy clones secreting the POIs (see Table 2) obtained and screened as described in Examples 3 and 4) were selected for bioreactor cultivations to further confirm the production performance of SP4 and SP14 without (Table 7) or with (Table 8) the MFα pro-sequence in comparison to the MFα secretion signal as control.

For the purpose of production of a POI and testing the secretion increasing effect of the signal peptides of the invention (SP4 (SEQ ID NO 2) and SP14 (SEQ ID NO 1), the best performing strains in screening with respect to POI titer, POI yield and growth are chosen for bioreactor cultivations, which may also be multicopy strains (i.e. they contain multiple copies of the POI expression cassette). Thus for expression and secretion of a POI by using the SP4 or SP14 alone without any pro-sequence or by using the SP4 or SP14 with an MFα pro-sequence the vectors and strains are generated and selected as described in Examples 2, 3, 4 and 5 and cultivated as described in Example 6, except that after screening as described in Example 4 the best performing strain or strains are selected for fermentation as described in Example 6. This also applies to the respective control strains, expressing the respective POI by using the MFα secretion signal (SEQ ID NO: 4). Titer and Yield Fold Change of the expression/secretion of the POI by using the SP4 or SP14 with or without an MFα pro-sequence related to the expression/secretion by using the MFα secretion signal (SEQ ID NO: 4) are calculated.

With the bioreactor cultivations, we confirmed that SP4 and SP14 combined with an MFα pro-sequence, clearly improved secretion capacity compared to the MFα secretion signal (SEQ ID NO: 4), as can be seen for POI titers and yields (Table 10). Again, there was no strong difference seen between the WT and the SRP overexpression strain background, albeit scR secretion levels with SP4 were higher in the SRP overexpressing strain than in the WT, indicating that SRP overexpression brings a benefit for some POIs.

TABLE 10
Bioreactor results obtained by using the candidate SPs fused
to an MFα pro-sequence for the secretion of VHH, scR
or SDZ-Fab as reporter POIs compared to using the MFα
secretion signal in either the CBS7435 mutS WT or the SRP
background (CBS7435 mutS SRP#7). Titer and yield FCs
were calculated compared to the MFα secretion signal
control by using the final sampling point.

| Background | Protein | Pre | Pro | Titer FC | Yield FC |
|---|---|---|---|---|---|
| SRP | VHH | SP4 | MFα-pro | 1.3 | 1.5 |
| WT | VHH | SP14 | MFα-pro | 1.4 | 1.5 |
| SRP | scR | SP4 | MFα-pro | 1.4 | 1.4 |
| SRP | scR | SP14 | MFα-pro | 1.2 | 1.2 |
| WT | scR | SP4 | MFα-pro | 1.1 | 1.1 |
| WT | SDZ-Fab | SP14 | MFα-pro | 1.8 | 1.9 |

### Example 9: Confirmation of Co-Translational Mode of Translocation When Using the Novel Signal Peptide Sequences

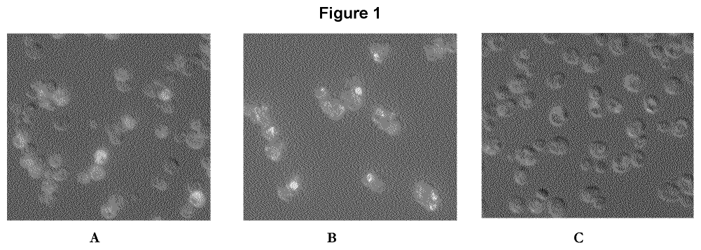

Immunofluorescence microscopy was used to confirm the co-translational translocation mode of SP4 and SP14. P. pastoris strains producing VHH with SP4 or SP14 were used for the microscopy analysis (see Example 2 for the generation of the strains). A mouse-anti 6XHis antibody (abcam, ab18184) and an appropriate fluorescently labelled secondary antibody were used for the immunofluorescence microscopy. While the clones using the standard MFαsecretion signal showed a prevalently cytosolic pattern indicative of post-translational translocation (FIG. 1A), both SP4-VHH and SP14-VHH led to a typical ER pattern (ring around the nucleus), which is indicative for the co-translational translocation mode (FIG. 1B-C). This confirms that they are preferentially translocated by the co-translational machinery.

### REFERENCES

Aw & Polizzi. Microb Cell Fact. 2013. 12, 128.
Ecker et al. MAbs. 2015. 7, 9-14.
Feige et al. Trends Biochem Sci. 2010. 35, 189-89
Fitzgerald & Glick. Microb Cell Fact. 2014. 13, 125.
Gasser et al. Future Microbiol. 2013. 8 (2), 191-208.
Janda et al. Nature. 2010. 465, 507-510.
Kyte & Doolittle. J Mol Biol. 1982. 157(1), 105-32.
Lin-Cereghino et al. Gene. 2013. 519, 311-7.
Looser et al. Biotechnol Adv. 2015. 33, 1177-93.
Nelson & Reichert. Nat Biotechnol. 2009. 27, 331-7.
Ng et al. The Journal of cell biology. 1996. 134 (2), 269-78.
Nielsen. Methods Mol Biol. 2017. 1611, 59-73.
Pechmann et al. Nat Struct Mol Biol. 2014. 21, 1100-5.
Pfeffer et al. Microb Cell Fact. 2011. 10, 47.
Prielhofer et al. BMC Syst Biol. 2017. 11, 123.
Prielhofer et al. BMC Genomics. 2015. 16, 167.
Rapoport. Nature. 2007. 450, 663-669.
Schwarzhans et al. Microb Cell Fact. 2016. 15, 84.
Sharp and Li. Nucleic Acids Res. 1987. 15, 1281-95.
Sturmberger et al. Journal of biotechnology. 2016. 235, 121-31.
Valli et al. FEMS Yeast Research. 2016. 16 (6) 2016.
Walsh. Nat Biotechnol. 2014. 32, 992-1000.
Zahrl et al. Microbiology. 2018.

1. A nucleic acid molecule encoding a fusion protein comprising from N-terminus to C-terminus
(a) a secretion signal, the secretion signal comprising
(I) (i) a signal peptide sequence originating from a KRE1 protein or a signal peptide sequence originating from a SWP1 protein; and
(ii) an α-mating factor (MFα) pro-sequence;
or
(II) a signal peptide sequence originating from a KRE1 protein or a signal peptide sequence originating from a SWP1 protein; and
(b) a protein of interest.

2. The nucleic acid molecule of claim 1, wherein the secretion signal increases secretion of said protein of interest from a eukaryotic host cell in comparison to said eukaryotic host cell expressing the nucleic acid molecule as defined in claim 1 but comprising a wild type Saccharomyces cerevisiae α-mating factor secretion signal (such as SEQ ID NO: 4) instead of the secretion signal as defined in claim 1.

3. The nucleic acid molecule of claim 1 or 2, wherein the signal peptide sequence originating from a KRE1 protein comprises SEQ ID NO: 1 or a functional homolog thereof.

4. The nucleic acid molecule of claim 1 or 2, wherein the signal peptide sequence originating from a SWP1 protein comprises SEQ ID NO: 2 or 52 or a functional homolog thereof.

5. The nucleic acid molecule of any one of the preceding claims, wherein the MFα pro-sequence comprises any one of SEQ ID NO: 3, 53 or 74-80 or a functional homolog thereof, preferably SEQ ID NO: 3 or 53 or a functional homolog thereof.

6. The nucleic acid molecule of any one of the preceding claims, wherein the MFα pro-sequence comprises Ser at a position corresponding to position 23 of SEQ ID NO: 53-and/or Glu at a position corresponding to position 64 of SEQ ID NO: 53.

7. The nucleic acid molecule of any one of claims 1 to 6, wherein the protein of interest is selected from the group consisting of an antibody such as a chimeric, humanized or human antibody, or a bispecific antibody, or an antigen-binding antibody fragment such as Fab or F(ab)2, single chain antibodies such as scFv, single domain antibodies such as VHH fragments of camelid or heavy chain antibodies or domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.

8. A secretion signal as defined in any one of claims 1 to 7.

9. An expression cassette or a vector comprising the nucleic acid molecule of any one of claims 1 to 7 and a promoter operably linked thereto.

10. A recombinant eukaryotic host cell comprising the nucleic acid molecule of any one of claims 1 to 7, or the expression cassette of claim 9 or the vector of claim 9.

11. The recombinant eukaryotic host cell of claim 10, wherein the host cell is a fungal or yeast host cell.

12. The recombinant eukaryotic host cell of claim 11, wherein the yeast host cell is selected from the group consisting of Komagataella phaffii (Pichia pastoris), Hansenula polymorpha, Saccharomyces cerevisiae, Saccharomyces paradoxus, Saccharomyces eubayanus, Saccharomyces kudriavzevii, Saccharomyces kluyveri, Saccharomyces uvarum, Kluyveromyces lactis, Yarrowia lipolytica, Pichia methanolica, Candida Komagataella spp. and Schizosaccharomyces pombe, or wherein the fungal host cell is selected from Trichoderma reesei or Aspergillus niger.

13. The recombinant eukaryotic host cell of any one of claims 10 to 12, wherein the host cell is engineered to overexpress one or more component(s) of the signal recognition particle (SRP).

14. A method of manufacturing a protein of interest in a eukaryotic host cell, comprising
(i) genetically engineering the eukaryotic host cell with the nucleic acid molecule of any one of claims 1 to 7 or with the expression cassette or vector of claim 9, and optionally genetically engineering the eukaryotic host cell to overexpress one or more component(s) of a signal recognition particle (SRP);
(ii) culturing the genetically engineered host cell under conditions to express the nucleic acid molecule and optionally to overexpress the one or more component(s) of the SRP, and to secrete the protein of interest upon cleavage of the secretion signal,
(iii) optionally isolating the protein of interest from the cell culture,
(iv) optionally purifying the protein of interest,
(v) optionally modifying the protein of interest, and
(vi) optionally formulating the protein of interest.

15. A method of increasing the secretion of a protein of interest from a eukaryotic host cell, comprising expressing in said eukaryotic host cell a nucleic acid molecule as defined in any one of claims 1 to 7 and optionally engineering the eukaryotic host cell to overexpress one or more component(s) of the signal recognition particle (SRP), thereby increasing the secretion of said protein of interest in comparison to said host cell expressing the nucleic acid molecule of any one of claims 1 to 7 comprising a wild type Saccharomyces cerevisiae α-mating factor secretion signal (such as SEQ ID NO: 4) instead of the secretion signal as defined in any of claims 1 to 7.

16. The method of claim 14 or 15, comprising
(i) engineering said host cell to incorporate an expression construct to express a nucleic acid molecule of any one of claims 1 to 7, and optionally genetically engineering the host cell to overexpress one or more component(s) of a signal recognition particle (SRP),
(ii) culturing said host cell under conditions suitable to express said nucleic acid molecule and optionally to overexpress the one or more component(s) of the SRP, and to secrete the protein of interest upon cleavage of the secretion signal,
(iii) optionally isolating the protein of interest from the cell culture,
(iv) optionally purifying the protein of interest,
(v) optionally modifying the protein of interest, and
(vi) optionally formulating the protein of interest.

17. The method of any one of claims 14 to 16, wherein the nucleic acid molecule is integrated in a chromosome of said host cell or contained in an expression cassette, a vector or plasmid, which does not integrate into the genome of said host cell.

18. The method of any one of claims 14 to 17, wherein the eukaryotic host cell is a fungal or yeast host cell.

19. The method of any one of claims 14 to 18, wherein the yeast host cell is selected from the group consisting of Komagataella phaffii (Pichia pastoris), Hansenula polymorpha, Saccharomyces cerevisiae, Kluyveromyces lactis, Yarrowia lipolytica, Pichia methanolica, Candida boidinii, Komagataella spp. and Schizosaccharomyces pombe, or wherein the fungal host cell is selected from Trichoderma reesei or Aspergillus niger.

20. The method of any one of claims 14 to 19, wherein the protein of interest is selected from the group consisting of an antibody such as a chimeric, humanized or human antibody, or a bispecific antibody, or an antigen-binding antibody fragment such as Fab or F(ab)2, single chain antibodies such as scFv, single domain antibodies such as VHH fragments of camelid or heavy chain antibodies or domain antibodies (dAbs), an artificial antigen-binding molecule such as a DARPIN, ibody, affibody, humabody, or a mutein based on a polypeptide of the lipocalin family, an enzyme such as a process enzyme, a cytokine, growth factor, hormone, protein antibiotic, fusion protein such as a toxin-fusion protein, a structural protein, a regulatory protein, and a vaccine antigen, preferably wherein the protein of interest is a therapeutic protein, a food additive or a feed additive.

21. Use of the secretion signal as defined in any one of claims 1 to 8 for increasing the secretion of a protein of interest from a eukaryotic host cell.

22. The use of claim 21, wherein the secretion signal increases secretion of said protein of interest from the eukaryotic host cell in comparison to said eukaryotic host cell expressing the fusion protein as defined in claim 1 comprising the wild type Saccharomyces cerevisiae α-mating factor secretion signal (such as SEQ ID NO: 4) instead of the secretion signal as defined in claim 1.

23. Use of the recombinant eukaryotic host cell of any one of claims 10 to 13 for manufacturing a protein of interest.

24. The fusion protein of any one of claims 1 to 7, wherein the signal peptide sequence is originating from a KRE1 protein.

25. The fusion protein of any one of claims 1 to 7, wherein the signal peptide sequence is originating from a SWP1 protein.

26. A secretion signal, the secretion signal comprising
(i) a signal peptide sequence originating from a KRE1 protein; and
(ii) an α-mating factor (MFα) pro-sequence.

27. A secretion signal, the secretion signal comprising
(i) a signal peptide sequence originating from a SWP1 protein; and
(ii) an α-mating factor (MFα) pro-sequence.

28. A method of producing a protein of interest by culturing the recombinant eukaryotic host cell of any one of claims 10 to 13 under conditions to express the nucleic acid molecule of any one of claims 1 to 7 and to secrete the protein of interest upon cleavage of the secretion signal, and isolating the protein of interest from the host cell culture and optionally purifying and optionally modifying and optionally formulating the protein of interest.


SEQUENCE LISTING (80 sequences)

SEQ ID NO: 1 - protein, 18 aa, Komagataella phaffii
MLNKLFIAILIVITAVIG

SEQ ID NO: 2 - protein, 18 aa, Komagataella phaffii
MKLISVGIVTTLLTLASC

SEQ ID NO: 3 - protein, 66 aa, Artificial sequence
  Pro-sequence of alpha-MF including  L23S, D64E
APVNTTTEDETAQIPAEAVIGYSDLEGDFDVAVLPFSNSTNNGLLFINTTIASIAAKEEGVSLEKR

SEQ ID NO: 4 - protein, 85 aa, Artificial sequence
  alpha-MF (pre-pro-sequence)  including  L23S, D64E
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 5 - protein, 567 aa, Komagataella phaffii
MESPLQSTYGERAERYLDSADAFHKQRHRLNRRLHKLRKSLDIHVTDTKNYREKEQISKIDLESYNRDKRYGDIILFTAERDHMYSDEVKEIMKVHHSKSREKFIVSRLKRSLDHGRKLLILVGDEPDEMRKLEVFVYVALIQGKLSIANKNWTNAQYALSVARCGLQFLDKYGTETQTDLYNGIIDTHIDQMLKFVIYQATKNNSPILDTECRHQIRTDTLGYLDQARQIIESKDPEFLNVGVVETQLIWWDYDISIHSEEVAKLISDANEKLQLIEDGNVSSYDPALLTLQEALDAHQLLMARNVDNFADDDQNNHVLLSYIRYLLLITTLRRDITLIDQVRNRSVVNSSLAVALERAKDVGRIFDNIVKKVNELKDVPGVYNKQEEWNSLQALDAYFQASKIQHLASTHLLFNRSKESLALLIKAKSLVKGHTIAGEYPTNFPTNKDLSSILEQINQDILKAYVLAKYKQESSLGGVSEYDFIADNRNKVPSNPSLHKIASVSYKNVKPVNVKPVLFDIAFNYVSQPNQIFEEPIESSNKQERQADSESPSPEKKKKGLFGLFR

SEQ ID NO: 6 - protein, 605 aa, Komagataella phaffii
MSSLSELVSELAIHSEKRQYKEAYEKAKRIIDLGHPLDLDTLKLGLVASINLDQYHNAGRLISKSKDHIVYDGMKEFLLLIGYVYYKNGDSKNFETLLKDSAFQGRAFEHLKAQYYYKIGENEKALKIYRELSKNPLDEVVDLSVNERAVISQLLELDGVVEQPVSRPIDDSYDCKFNDALYQVKIGDYESALDLLEEAKAICEENTKDLPLDTREAEIVPILLQIAYVKQLKGKKEESLTALRSLSKPKDSLLDLIYRNNLLSLRIDEYGRNDTNFHILYRELGFPNSIDINKDKLTVSQRVALTRNESLLALELGKIPSQSDLKLFYDATSEFLDLNTKLEASMIYNYFMRRPGQQEVPNALLTAQLAINVGNINNARTVLETVVSNDEKNLLEPSIVVSLYLIYDKLQSGRLQVELLKKVADLLLESEISSTQQRKFFKDIAFKTLNHDAVLANRLFEKLHSIYPNDELVSTYLNSSSNASNNNTTTTNFSELDDLVLGIDTDKLISEGFDTFESSKRPTTIISSTNKKRRTRLKPKHEAKEKYKRLDEERWLPLKDRSYYRPKKGKKIRNTTQGTVTSNTSEISGLKKTLPKKSSKKKGRK

SEQ ID NO: 7 - protein, 151 aa, Komagataella phaffii
MPPVKSLDIFFNRTEKLLEANPTTTKVSIKLGVNFNDHENPQSKHNVITVRVSDPVSGSNFKFKVTNKTDMLKIFSFLGPHGIELPISGQQSQIKSNDQTQSDNTEVPTTFHRGATSILANKAFEKKPLIIKDSSTAKKGGKGGKKKGKKF

SEQ ID NO: 8 - protein, 270 aa, Komagataella phaffii
MPLLEEISDAEDIDNLEMDLAEFDPTLRTPIAEQRPAPQVVRSQDAESGQTPLVPNQDQISQYIEQFKEGGTINKDQVIRPDEMMEKEMAELKSFQILYPCYFDKNRSVKEGRRCQKEYGVENPLAKTILDACRYLDIPCILEPEKTHPQDFGNPGRVRVAIKESGKYLDEQYKTKRKLIQLVGQFLVEHPTTLQKVQELPGPPELQQGGYIPERVPRVKGLKMNEIVPLHSPFTIKHPSTKSVYEREPEPAPPAAVPKAPKQKKIMVRR

SEQ ID NO: 9 - protein, 518 aa, Komagataella phaffii
MVLADLGRRINNAVGNVTKSNVVDADVISNMLKEICNALLESDVNIKLVAQLREKIRKQIDAEDKPGINKKKLIQKVVFDELVKLVDCNEAELFKPKKKQTNVIMMVGLQGAGKTTTCTKLAVYYQRRGFKVGMVCGDTFRAGAFDQLKQNATKAKIPYYGSYTETDPVKVTFDGVEEFRKEKFEIIIVDTSGRHRQEEDLFEEMVQIGKAIKPNQTIMVLDASIGQSAESQSKAFKESSDFGAIIITKMDSNSKGGGALSAIAATNTPVAFIATGEHIQNFEKFSGRGFISKLLGIGDIEGLMEHVQSMNLDQGDTIKNFKEGKFTLQDFQTQLNNIMKMGPLSKLAQMLPGGMGQLMGQVGEEEASKRLKRMIYIMDSMTKQELSSDGRLFIDQPSRMVRVARGSGTSVTEVELVLLQQKMMARMALQSKNMMSGAGGPAGMASKMNPANMRRAMQQMQSNPGMMDNMMNMFGGAGGAGGAGMPDMQEMMKQMSSGQMKMPSQQEMMSMMKQFGMG

SEQ ID NO: 10 - protein, 151 aa, Komagataella phaffii
MSTTTKKNKNRILIENHKQFLEEVSKTATLSVWNSKFSIKRLSLEADPVEGTPEGIRDIPQGVETNSIIGNSVENDSKSHPILFRYTARHAKEKIPEVRISTTVDSEQLSTFWRDYVDILKGSSQLKLQSETKKVSSKKSKAKKKRGKGAW

SEQ ID NO: 11 - DNA, 323 bp, Komagataella phaffii
atgcagcctctgatgagtccgtgaggacgaaacgagtaagctcgtcaggctgttatggcgcatccgggggaggtagttacttgaccttgattcctaatagcttacaactgaggtgtctcgttcgatcctggcggtccgcaatattttccatacgagtaatctgtgggggaaggcgagcaataagacgtgccaccgcccaaggggagcaatccagcagggaacacgtcccgcaaggaggcgggtgagatagcatctcgttggtaatgggctgttggtgaacaaagtttgactatgtgaaccggctatttacatttttgcttttt

SEQ ID NO: 12 - DNA, 1707 bp, Komagataella phaffii
atggaatcgcccttgcaatctacatacggagaaagagccgaaaggtatttagatagtgctgatgcttttcataaacaaagacacagattgaatcgaaggctgcacaagttacgtaagagcttggatattcatgttactgatactaagaactatagagagaaagagcagatttccaaaattgatctagagtcgtacaacagggataagcgatatggtgacattatactgttcactgcagagagggatcacatgtatagtgatgaggtcaaggagatcatgaaggtccatcatagtaaatcgagagaaaagtttattgtttctagattgaagagatcactggaccacggtagaaaattactgatcctagttggagacgagcctgatgagatgagaaaattggaagtatttgtttatgttgcattgattcagggtaaactttccattgcaaacaagaattggaccaatgctcagtatgctctcagtgtggcgagatgtgggctccagtttttggacaaatatggtactgaaacacaaactgacctctataatggcataattgacactcacatagatcaaatgttgaaatttgtgatctaccaagctactaaaaataacagtcctattttggatacagagtgcagacatcaaattaggacggacaccctagggtatttggatcaggcaaggcaaataatagaatcaaaagatcccgagtttctgaatgttggagttgttgaaactcagttgatttggtgggactacgatatctctattcattcagaggaggtagcaaagctgatttcagatgcgaacgaaaagctgcaacttatcgaggatggaaacgtctcctcatatgatccggctctactaactcttcaagaagcgctggatgctcatcagttgttgatggccagaaatgttgacaacttcgcagacgacgatcaaaacaatcatgttttactgtcgtacatcagatatttgttacttatcaccactttgagaagggacattactttgatagaccaagttagaaacagatctgtggttaattcttccctagctgtggctctggaacgtgctaaagacgttggtagaattttcgacaatatcgtcaagaaagtcaatgagttgaaagacgttccaggtgtttacaacaagcaagaggagtggaattcgttgcaggcattggatgcttatttccaagcatccaagatccaacatttggcatctacccaccttttattcaacagatccaaggaatcattggcgttattaataaaggcaaagtccttggtaaaggggcacactatcgccggagaatatcccactaatttccctacgaataaagatttgagttcgatcttagaacaaattaatcaagacatccttaaggcttatgttttggccaagtataagcaagagtcctctttaggtggtgtatcggagtatgatttcattgctgacaatcgcaacaaggttccgtcgaatcccagtctgcacaagattgcctctgtatcctacaagaatgtcaaacctgtcaatgttaagcctgtattgtttgacatagctttcaactacgtgagtcaaccaaaccagatatttgaggaacctatcgagagttcaaacaagcaagagagacaagcggattctgaatctccgtcaccagagaagaaaaaaaagggattgtttggattgttccgctagtag

SEQ ID NO: 13 - DNA, 1821 bp, Komagataella phaffii
atgtcgtctctttcagagctggtatcggaattggcaatccattctgagaagaggcagtacaaagaagcatatgagaaagcaaagcgcattatagatttgggccaccctcttgaccttgacacattgaagctaggtttggtggcttcgatcaacctggaccaatatcacaacgcaggtcgcctcatatcaaaaagtaaggatcatatcgtatatgatggaatgaaggaattcttgctattaattggatatgtgtactacaagaacggagactcgaagaattttgaaactctactaaaggattcagctttccaaggaagagcatttgaacacctcaaagcccaatactattacaagatcggggagaacgaaaaggcactcaagatttaccgagagctatctaagaacccattggatgaagttgtagacttgagtgtcaatgaaagagctgtcattagccagcttttggaattggatggtgtcgttgaacagcctgtatctcgaccaatagacgactcatacgattgcaaattcaacgatgccctttaccaggtaaagattggtgactatgaatctgcattggatctcttggaagaagccaaagccatatgtgaagaaaatacaaaagatctgcctttggacacacgagaagcagagattgttcctattctgttacaaattgcttacgtcaaacaacttaagggtaaaaaggaagaatccttgactgcgctgagaagtctttctaaaccaaaggactctcttttagatcttatttacagaaataacttactgtcattaaggattgatgaatacggaagaaatgataccaactttcatattctttatcgtgagttaggattccctaattcgatagacattaataaagacaagttgacagtgtcccaaagggttgcgttgaccagaaatgaatcattattggcactcgagcttggaaagatcccatctcaaagtgatctcaagctcttttatgatgctacttcggaatttttagatttgaacaccaagctagaagcctcaatgatttataattatttcatgagacgccctggccagcaagaggttccaaatgctcttttaactgcacagctggctatcaacgttggtaacatcaataatgcaagaactgttcttgaaactgtggtgtcgaacgacgaaaagaatttactagaaccatctattgtggtatctttgtatttgatttacgataagcttcaaagcggaagattgcaagttgaactcctgaagaaagtagcggatcttttgctagaatcagagatttccagcactcaacaacgtaagtttttcaaagatattgccttcaaaaccctcaatcacgatgcagttttggccaatcgactatttgagaaactgcatagtatttaccctaatgatgagttggtatccacgtacttgaactcttcatctaatgcatccaacaataacactaccacgaccaacttctccgaattggacgacttggtcctaggcatagacacggataagcttattagtgaaggatttgatacttttgagtccagcaaaagaccgaccacgattatcagctcaactaacaagaaacgtcgtactagattgaagcccaaacatgaagccaaggaaaagtataagcgtctggatgaggaaagatggttacctttaaaggaccgcagttattacagacccaaaaagggaaagaaaattagaaataccactcagggtactgtcactagtaatactagtgaaataagtggcttgaaaaagactctgccaaagaaaagttccaaaaagaaaggaagaaaatgatag

SEQ ID NO: 14 - DNA, 456 bp, Komagataella phaffii
atgcctcctgtgaaatctctggacatctttttcaaccgcacagagaagctcttagaagccaaccccacaacgacaaaagtttccatcaaattgggcgtaaatttcaatgatcacgagaatcctcaaagcaagcacaacgtcataacggtgagagtatctgatccagtgagcgggtccaatttcaaattcaaagtgaccaataaaactgatatgctgaaaatattcagtttcttaggtcctcatggcattgagttaccaatttctggccagcaaagccagataaagagtaatgatcagactcagagtgacaatactgaagtgcctaccacatttcataggggagccaccagtattttggctaataaggcatttgagaagaaaccactgattattaaggattcaagtaccgcaaagaaaggtggtaaaggtggtaagaagaagggtaagaaattttaa

SEQ ID NO: 15 - DNA, 816 bp, Komagataella phaffii
atgccattactagaggaaataagtgatgcagaggacatagacaacttggagatggatttagccgagtttgatcctactttaaggactccgatagctgagcaaagaccagctcctcaggttgtcagatcacaagatgccgaaagtggacagactcctttggttcctaaccaggatcaaataagtcagtatattgaacaattcaaagaaggtggcaccataaacaaggatcaagtgattagacccgacgaaatgatggaaaaagaaatggcagagttgaaaagcttccaaattttgtacccatgttactttgataaaaatagaagtgttaaagaaggaagaagatgccaaaaggagtatggtgtggagaaccccctggcaaagacaatattagatgcttgcaggtacttggatataccttgcatcctggagcctgaaaagactcatcctcaagattttggtaatccaggaagagtgagagtggctatcaaggagagtgggaagtatctcgatgaacaatataagaccaaaaggaaactaatacagttggtaggacaatttctggttgaacatccaacaacgttacagaaagttcaagaattgcccggtccacctgagttgcaacagggcgggtacattccagaacgtgtaccccgagtgaaagggttaaagatgaacgaaattgttcctttgcattcgccattcactattaagcatccaagtactaaatctgtttatgaaagggaacctgagcccgcaccacccgccgccgtgcccaaagctccgaaacagaagaaaataatggtgagaagataatag

SEQ ID NO: 16 - DNA, 1560 bp, Komagataella phaffii
atggtattggcagatcttggaaggcgtatcaataacgccgttggaaatgtcaccaagtccaatgttgttgacgctgacgtcatcagcaacatgttaaaggagatttgtaacgccctattggagtccgatgtgaacattaaactagttgcccaattgagagagaaaatacgaaaacagatcgacgcagaggataaaccaggaattaataagaagaagctgatccagaaggtcgtttttgatgagctggtgaaacttgttgattgcaacgaagctgagctgttcaagccaaagaaaaaacagacgaatgtgatcatgatggtcggtttacaaggtgctggtaagacaacaacctgtactaaactggcagtgtattaccagagaagaggattcaaagtgggaatggtctgtggtgacactttccgagctggtgcgtttgaccagctgaaacaaaacgctaccaaggctaagattccctactatggttcatatacagaaactgaccctgtgaaagtgacctttgatggtgtggaagaattcaggaaggaaaagtttgaaataataattgtggatacttctggtagacacaggcaggaggaagatttattcgaagagatggtacaaattggaaaagctatcaagcctaatcaaacaatcatggtactggatgcttccataggtcaatctgccgaatctcaatctaaagcatttaaggaatcatccgattttggtgccattatcataactaaaatggattccaattccaagggaggaggtgccctttcagctatagctgccaccaacactccagtagcgtttattgccaccggagagcacattcagaatttcgaaaagttttcaggaagaggatttatctcaaaacttttaggaattggtgatatagagggtcttatggaacatgttcagtcgatgaacttggatcaaggtgatactatcaagaatttcaaggaaggaaagtttactttacaggattttcaaacgcaattgaacaacatcatgaagatggggccactgtccaaactcgctcaaatgttgcctggtggaatgggacaattgatgggacaggttggtgaagaggaggcttcaaagagattgaagcgaatgatttatataatggattcaatgacgaagcaagagttgtcaagtgacggtagattgtttattgatcagccttcaaggatggtaagagttgctagaggctctggtacctctgtaactgaggtggagcttgttcttttacagcaaaagatgatggctcgtatggcattacaatctaagaatatgatgagtggggccggcggtccagcagggatggcttccaaaatgaatccagctaatatgagaagagctatgcaacaaatgcaatcaaacccaggaatgatggataacatgatgaatatgtttggtggagctggaggagctggaggagctggaatgccggatatgcaagaaatgatgaagcaaatgtccagtggccaaatgaaaatgcccagtcaacaggaaatgatgagcatgatgaaacagtttggtatgggctaatag

SEQ ID NO: 17 - DNA, 456 bp, Komagataella phaffii
atgtccacaactactaagaaaaacaagaacaggatcttgatagagaatcacaaacagttcctggaagaagtttccaaaacagccactttatcagtttggaactcgaaattttcaatcaaacgactgtctctagaagcagatcccgtggaagggacgcctgaaggaatcagagatatcccacaaggagtagagacaaactctataataggaaacagcgtagagaatgattcaaagtcacaccccattttattcagatatacagctagacacgcaaaggagaaaataccagaggtgcgaatttcaaccaccgttgattcagagcagctaagcaccttctggagagattatgtggacatattgaagggaagctcccaattgaaactgcagtcagaaaccaagaaagtcagtagtaagaagagcaaggcgaagaagaagagaggaaagggtgcatggtaa

SEQ ID NO: 18 - DNA, 54 bp, Komagataella phaffii
atgaagctgatctccgtgggtatagtgacgacattactgactttggccagttgc

SEQ ID NO: 19 - DNA, 54 bp, Komagataella phaffii
atgttaaacaagctgttcattgcaatactcatagtcatcactgctgtcataggc

SEQ ID NO: 20 - DNA, 198 bp, Artificial
  Pro-sequence of alpha-MF including  L23S, D64E
gcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagaga

SEQ ID NO: 21 - DNA, 255 bp, Artificial
  alpha-MF (pre-pro-sequence)  including  L23S, D64E
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagaga

SEQ ID NO: 22 - DNA, 837 bp, Artificial
  vHH protein
caggttcagctgcaggagtccggtggtggtctggttcaagccggtggttcattaagattgtcctgtgctgcctctggtagaactttcacttctttcgcaatgggttggtttagacaagcacctggaaaagagagagagtttgttgcttctatctccagatccggtactttaactagatacgctgactctgccaagggtagattcactatttctgttgacaacgccaagaacactgtttctttgcaaatggacaaccttaacccagatgacaccgcagtctattactgtgccgctgacttgcacagaccatacggtccaggaacccaaagatccgatgagtacgattcttggggtcagggaactcaagtcactgtctcttcaggtggtggatctggtggtggaggttcaggtggtggaggatccggtggtggtggttctggtggtggtggatctggtggaggtgaagttcaacttgtcgaatccggtggtgcacttgtccaacctggtggatctcttagactttcttgtgccgcctccggttttcctgttaaccgttactctatgcgttggtacagacaagcccctggaaaagaacgtgaatgggttgccggaatgtcctcagctggtgacagatcctcctacgaagattctgtgaagggacgtttcaccatctccagagatgacgcccgtaacaccgtttaccttcaaatgaactcccttaagcctgaggatactgccgtctactattgtaacgtgaatgtcggatttgaatactggggacagggaacccaagttactgtctcttccggtggacatcaccaccaccatcactaatag

SEQ ID NO: 23 - DNA, 774 bp, Artificial
  scR protein
caggaacaactaatggagtctgggggtggtttggttaccctgggtggttctcttaagctttcatgtaaggcctctggtattgatttttcgcactacggtatctcctgggttagacaagctcctggaaaaggtctggaatggatcgcttacatttacccaaattacggttctgttgactatgcctcctgggtcaatggtaggttcactatttcccttgacaacgctcagaacacggtattcctacagatgatctccctaaccgctgctgatactgcaacctacttctgtgctcgtgacagaggttactactctggctctcgtggaactagacttgacttatggggacaaggtactctcgttaccatctctagtggtggaggtggttctggaggaggaggttccggcggaggtggtagcgagctggtcatgactcaaacccctccatccctatctgcatcagtcggtgaaaccgttagaattagatgccttgcatctgagttcttgttcaacggtgtgtcctggtatcaacaaaagcctggtaagcctccaaagtttctcatttctggtgcctcaaacctcgaatctggagtgccaccaagattttccggatctggctctggtactgactacactctgacaattggtggtgttcaagctgaggatgttgctacctactattgtctcggtggttactcaggatcttccggcctaactttcggtgccggtacaaacgtcgagatcaaaggtggacatcaccaccaccatcactaatag

SEQ ID NO: 24 - DNA, 687 bp, Artificial
  SDZ-Fab-HC
gaggtccaattggtccaatctggtggaggattggttcaaccaggtggatctctgagattgtcttgtgctgcttctggtttcaccttctctcactactggatgtcatgggttagacaagctcctggtaagggtttggaatgggttgctaacatcgagcaagatggatcagagaagtactacgttgactctgttaagggaagattcactatttcccgtgataacgccaagaactccttgtacctgcaaatgaactcccttagagctgaggatactgctgtctacttctgtgctagagacttggaaggtttgcatggtgatggttacttcgacttatggggtagaggtactcttgtcaccgtttcatctgcctctaccaaaggaccttctgtgttcccattagctccatgttccagatccacctccgaatctactgcagctttgggttgtttggtgaaggactactttcctgaaccagtgactgtctcttggaactctggtgctttgacttctggtgttcacacctttcctgcagttttgcagtcatctggtctgtactctctgtcctcagttgtcactgttccttcctcatctcttggtaccaagacctacacttgcaacgttgaccataagccatccaataccaaggttgacaagagagttgagtccaagtatggtccaccttaatag

SEQ ID NO: 25 - DNA, 648 bp, Artificial
  SDZ-Fab-LC
gctatccagttgactcaatcaccatcctctttgtctgcttctgttggtgatagagtcatcctgacttgtcgtgcatctcaaggtgtttcctcagctttagcttggtaccaacaaaagccaggtaaagctccaaagttgctgatctacgacgcttcatcccttgaatctggtgttccttcacgtttctctggatctggatcaggtcctgatttcactctgactatctcatcccttcaaccagaggactttgctacctacttctgtcaacagttcaactcttaccctttgacctttggaggtggaactaagttggagatcaagagaactgttgctgcaccatcagtgttcatctttcctccatctgatgagcaactgaagtctggtactgcatctgttgtctgcttactgaacaacttctacccaagagaagctaaggtccaatggaaggttgacaatgccttgcaatctggtaactctcaagagtctgttactgagcaagactctaaggactctacttactccctttcttccaccttgactttgtctaaggctgattacgagaagcacaaggtttacgcttgtgaggttactcaccaaggtttgtcctctcctgttaccaagtctttcaacagaggtgaatgctaatag

SEQ ID NO: 26 - protein, 277 aa, Artificial
  vHH protein
QVQLQESGGGLVQAGGSLRLSCAASGRTFTSFAMGWFRQAPGKEREFVASISRSGTLTRYADSAKGRFTISVDNAKNTVSLQMDNLNPDDTAVYYCAADLHRPYGPGTQRSDEYDSWGQGTQVTVSSGGGSGGGGSGGGGSGGGGSGGGGSGGGEVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGHHHHHH

SEQ ID NO: 27 - protein, 256 aa, Artificial
  scR protein
QEQLMESGGGLVTLGGSLKLSCKASGIDFSHYGISWVRQAPGKGLEWIAYIYPNYGSVDYASWVNGRFTISLDNAQNTVFLQMISLTAADTATYFCARDRGYYSGSRGTRLDLWGQGTLVTISSGGGGSGGGGSGGGGSELVMTQTPPSLSASVGETVRIRCLASEFLFNGVSWYQQKPGKPPKFLISGASNLESGVPPRFSGSGSGTDYTLTIGGVQAEDVATYYCLGGYSGSSGLTFGAGTNVEIKGGHHHHHH

SEQ ID NO: 28 - protein, 227 aa, Artificial
  SDZ-Fab-HC
EVQLVQSGGGLVQPGGSLRLSCAASGFTFSHYWMSWVRQAPGKGLEWVANIEQDGSEKYYVDSVKGRFTISRDNAKNSLYLQMNSLRAEDTAVYFCARDLEGLHGDGYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 29 - protein, 214 aa, Artificial
  SDZ-Fab-LC
AIQLTQSPSSLSASVGDRVILTCRASQGVSSALAWYQQKPGKAPKLLIYDASSLESGVPSRFSGSGSGPDFTLTISSLQPEDFATYFCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 19 bp, Artificial
  SRP14 fwd
agacacgcaaaggagaaaa

SEQ ID NO: 31 - DNA, 22 bp, Artificial
  SRP14 rev
gtccacataatctctccagaag

SEQ ID NO: 32 - DNA, 19 bp, Artificial
  SRP21 fwd
ggggagccaccagtatttt

SEQ ID NO: 33 - DNA, 21 bp, Artificial
  SRP21 rev
ccacctttaccacctttcttt

SEQ ID NO: 34 - DNA, 19 bp, Artificial
  Sec65 fwd
ttcctttgcattcgccatt

SEQ ID NO: 35 - DNA, 21 bp, Artificial
  SEC65 rev
ttcttctgtttcggagctttg

SEQ ID NO: 36 - DNA, 19 bp, Artificial
  SRP54 fwd
aagatgatggctcgtatgg

SEQ ID NO: 37 - DNA, 20 bp, Artificial
  SRP54 rev
tcctgggtttgattgcattt

SEQ ID NO: 38 - DNA, 20 bp, Artificial
  SRP68 fwd
caactacgtgagtcaaccaa

SEQ ID NO: 39 - DNA, 19 bp, Artificial
  SRP68 rev
agcggaacaatccaaacaa

SEQ ID NO: 40 - DNA, 20 bp, Artificial
  SRP72 fwd
tgccttcaaaaccctcaatc

SEQ ID NO: 41 - DNA, 20 bp, Artificial
  SRP72 rev
ggtcgtggtagtgttattgt

SEQ ID NO: 42 - DNA, 18 bp, Artificial
  scpRNA fwd
gggaaggcgagcaataag

SEQ ID NO: 43 - DNA, 18 bp, Artificial
  scpRNA rev
accaacagcccattacca

SEQ ID NO: 44 - DNA, 22 bp, Artificial
  scR fwd
aagcctggtaagcctccaaagt

SEQ ID NO: 45 - DNA, 23 bp, Artificial
  scR rev
tcctcagcttgaacaccaccaat

SEQ ID NO: 46 - DNA, 22 bp, Artificial
  vHH fwd
tgtaacgtgaatgtcggatttg

SEQ ID NO: 47 - DNA, 20 bp, Artificial
  vHH rev
tagtgatggtggtggtgatg

SEQ ID NO: 48 - DNA, 24 bp, Artificial
  SDZ-Fab-HC fwd
tactgctgctttgggttgtttggt

SEQ ID NO: 49 - DNA, 24 bp, Artificial
  SDZ-Fab-HC rev
aagggacagtaacaacagaggaca

SEQ ID NO: 50 - DNA, 23 bp, Artificial
  SDZ-Fab-LC fwd
gatgaacaattgaagtctggtac

SEQ ID NO: 51 - DNA, 23 bp, Artificial
  SDZ-Fab-LC rev
gagtaacttcacaagcgtaaacc

SEQ ID NO: 52 - protein, 18 aa, Komagataella pastoris
MKLFFVGIVTTLLTLVSC

SEQ ID NO: 53 - protein, 66 aa, Saccharomyces cerevisiae
APVNTTTEDETAQIPAEAVIGYLDLEGDFDVAVLPFSNSTNNGLLFINTTIASIAAKEEGVSLDKR

SEQ ID NO: 54 - DNA, 741 bp, Artificial
  mCherry
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggagattacaaggatgacgatgataagtaatag

SEQ ID NO: 55 - protein, 245 aa, Artificial
  mCherry
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGDYKDDDDK

SEQ ID NO: 56 - protein, 16 aa, Komagataella phaffii
MLVAWFLLLLVSSCIC

SEQ ID NO: 57 - protein, 19 aa, Komagataella phaffii
MKFAISTLLIILQAAAVFA

SEQ ID NO: 58 - protein, 20 aa, Komagataella phaffii
MKFGLGSLGLAVALIPIASA

SEQ ID NO: 59 - protein, 18 aa, Komagataella phaffii
MIILLPLLFLFVAGLVQA

SEQ ID NO: 60 - protein, 16 aa, Komagataella phaffii
MDPFSILLTLTLIILA

SEQ ID NO: 61 - protein, 24 aa, Komagataella phaffii
MRLSYECLFSVFLVLAYHLKGTKA

SEQ ID NO: 62 - protein, 20 aa, Komagataella phaffii
MINLNSFLILTVTLLSPALA

SEQ ID NO: 63 - protein, 18 aa, Komagataella phaffii
MQLQYLAVLCALLLNVQS

SEQ ID NO: 64 - protein, 20 aa, Komagataella phaffii
MWIERNLIASILLFSTSAYA

SEQ ID NO: 65 - protein, 25 aa, Komagataella phaffii
MNISTASKISRLLQLVIALISLVLT

SEQ ID NO: 66 - protein, 20 aa, Komagataella phaffii
MFVFEPVLLAVLVASTCVTA

SEQ ID NO: 67 - DNA, 22 bp, Artificial
  mCherry forward
catcaagttggacatcacctcc

SEQ ID NO: 68 - DNA, 20 bp, Artificial
  mCherrey reverse
cacccttgtacagctcgtcc

SEQ ID NO: 69 - protein, 100 aa, Komagataella phaffii
LLIPSLDQLNIQLPFSLPHHTESPSLKLQGSNPFESSTVRPDPIQIYSTGYKVIENSYIVTVDSSITDSELQQLYDYIKGGYEFMLNNEDPFFVAMGIKR

SEQ ID NO: 70 - protein, 62 aa, Komagataella phaffii
CLQLLTTSIPPSFLTMVPEHYIGSKSVDEVPTSEDPRVNACPYICDIGDCSRGYSRASHLKR

SEQ ID NO: 71 - protein, 13 aa, Komagataella phaffii
RPLEHAHHQHDKR

SEQ ID NO: 72 - protein, 8 aa, Komagataella phaffii
YPLVIKKR

SEQ ID NO: 73 - protein, 2 aa, Komagataella phaffii
KR

SEQ ID NO: 74 - protein, 66 aa, Saccharomyces paradoxus
APVNTTTEDETAQIPAEAIIGYLDLEGDFDVAVLPFSNSTNNGLLFINTTIANIAAEEEGVTLNKR

SEQ ID NO: 75 - protein, 66 aa, Saccharomyces paradoxus
APVNTTTEDETAQIPAEAIIGYLDLEGDFDIAVLPFSNSTNNGLLFINTTIANIAAEEEGVTLNKR

SEQ ID NO: 76 - protein, 65 aa, Saccharomyces paradoxus
PVNTTTEDEMAQIPAEAIIGYLDLEGDFDVAVLPFSNSTNNGLLFINTTIANIAAEEEGVTLNKR

SEQ ID NO: 77 - protein, 65 aa, Saccharomyces paradoxus; identical to SEQ ID NO: 76

SEQ ID NO: 78 - protein, 65 aa, Saccharomyces paradoxus
PVNTTTEDEMARIPAEAIIGYLDLEGDFDVAVLPFSNSTNNGLLFINTTIANIAAEEEGVTLNKR

SEQ ID NO: 79 - protein, 66 aa, Saccharomyces eubayanus
APVNTTTENETAQIPAEAIIGYLDLEGDSDVAVLPFANSTNTGLLFINTTISNLAAKEEGVSLSKR

SEQ ID NO: 80 - protein, 66 aa, Saccharomyces kudriavzevii
APVNTTSESETVQIPAEAIIGYLDLEGDFDVAVLPFANSTNNGLLFINTTIANLATKEESVPLSKR